(19) United States
(12) Patent Application Publication
(10) Pub. No.: US 2025/0342933 A1
(43) Pub. Date: Nov. 6, 2025

(54) Pathology Modifying Neuromodulation Therapy Design

(71) Applicant: The Board of Trustees of the Leland Stanford Junior University, Stanford, CA (US)

(72) Inventors: Jin Hyung Lee, Palo Alto, CA (US); Ehsan Dadgar-Kiani, Redwood City, CA (US)

(21) Appl. No.: 19/112,427

(22) PCT Filed: Sep. 28, 2023
(86) PCT No.: PCT/US2023/075416

§ 371 Date: Mar. 17, 2025

Related U.S. Application Data
(60) Provisional application No. 63/377,932, filed on Sep. 30, 2022.

(51) Int. Cl. G16H 20/30; A61N 1/36; A61N 2/00; A61N 5/06; A61N 5/067; G06T 7/00; G06T 7/11; G06T 7/33; G06T 7/62; G06T 7/73

(52) U.S. Cl. CPC G16H 20/30; A61N 1/36139; A61N 5/062; A61N 5/067; G06T 7/0012; G06T 7/11; G06T 7/344; G06T 7/62; G06T 7/75; A61N 2/006; A61N 2005/063; A61N 2005/0663; G06T 2207/20021; G06T 2207/20081; G06T 2207/20084; G06T 2207/30016

(57) ABSTRACT

Methods, systems, and devices, including computer programs encoded on a computer storage medium are provided for optimizing neuro stimulation therapy for treatment of neurological and neurodegenerative diseases. In particular, an algorithm is used to provide a predicted regional pathological density map of neuropathology and predict locations of future spreading. Neurostimulation therapy parameters including the location, strength, and frequency of neuro stimulation can be adjusted accordingly to treat neuropathology and reduce aggregation and spreading.

### CROSS REFERENCE TO RELATED APPLICATION

This application claims benefit of U.S. Provisional Patent Application No. 63/377,932, filed Sep. 30, 2022, which application is incorporated herein by reference in its entirety.

### STATEMENT REGARDING FEDERALLY SPONSORED RESEARCH OR DEVELOPMENT

This invention was made with Government support under contracts NS091461, AG064051, AG047666, EB030884, MH114227, NS087159 and NS116783 awarded by the National Institutes of Health. The Government has certain rights in the invention.

### BACKGROUND OF THE INVENTION

An emerging hypothesis regarding the progression of neurodegenerative diseases is the spreading of misfolded proteins throughout the central nervous system (Goedert et al., 2013; Jucker and Walker, 2013; Oliveira et al., 2021). Building off the longstanding Braak hypothesis—which correlates progression of pathology with motor and cognitive symptoms (Braak et al., 2002; Braak et al., 2003)—small seeds of misfolded versions of disease proteins have been shown to spread from cell to cell, causing pathology and neurodegeneration in their wake (Volpicelli-Daley et al., 2011). Misfolding and spreading of the protein α-synuclein (α-syn) has been implicated in many neurodegenerative diseases, collectively referred to as synucleinopathies, including Parkinson's Disease (PD), Lewy Body Dementia (LBD), and Multiple System Atrophy (MSA) (Kordower et al., 2008; Lee and Trojanowski, 2006). In mouse models, even small seeds of injected pre-formed fibrils (PFF) of α-syn can recruit endogenous α-syn protein and trigger widespread pathology in a “prion-like” manner (Luk et al., 2012). Although the precise mechanism and genetic pathways involving whole brain pathogenesis resulting from small seeds of PFFs remains mostly unknown, many recent studies indicate that these fibrils spread along axonal pathways (Henderson et al., 2019; Henrich et al., 2020; Pandya et al., 2019).

### SUMMARY OF THE INVENTION

Methods, systems, and devices, including computer programs encoded on a computer storage medium are provided for optimizing neurostimulation therapy for treatment of neurological and neurodegenerative diseases. In particular, an algorithm is used to provide a predicted regional pathological density map of neuropathology and predict locations of future spreading. Neurostimulation therapy parameters including the location, strength, and frequency of neurostimulation can be adjusted accordingly to treat neuropathology and reduce aggregation and spreading.

In one aspect, a computer implemented method for predicting locations where pathological protein aggregates will develop in the brain of a subject who has a neurological or a neurodegenerative disease is provided, the computer performing steps comprising: a) receiving an image of the brain of the subject; b) identifying pathological protein aggregates in the image using a machine learning algorithm; c) mapping the positions of the pathological protein aggregates to neuroanatomical regions; d) modeling a discretized distribution of the pathological protein aggregates in each neuroanatomical region as a set of differential equations using a Smoluchowski network model; e) using regional gene density maps for each neuroanatomical region to calculate a distribution of gene effects on regional spreading and decay of the pathological protein aggregates; f) predicting changes in regional density of the pathological protein aggregates as a function of time by modeling spreading, aggregation, decay, and the spatial gene expression of the pathological protein aggregates throughout the neuroanatomical regions, wherein spreading is assumed to occur retrogradely between anatomically interconnected neuroanatomical regions, wherein spreading is modeled as diffusion through a weighted directed graph connecting the neuroanatomical regions of the brain of the subject; and g) predicting past locations, present locations, and future locations of the pathological protein aggregates based on said modeling.

In certain embodiments, the method further comprises adjusting one or more programmed neurostimulation parameters based on said predicting the changes in regional density of the pathological protein aggregates as a function of time. For example, the duration, amplitude, frequency, pulse width, and location of the neurostimulation, or any combination thereof may be adjusted.

In certain embodiments, the method further comprises instructing a neurostimulation device to apply neurostimulation to locations of the brain where pathological protein aggregates are predicted to be present in order to treat the neurological or neurodegenerative disease in the subject.

In certain embodiments, the method further comprises instructing the neurostimulation device to apply electrical stimulation to locations of the brain where pathological protein aggregates are not yet present but are predicted to occur at a future time based on said predicting changes in the regional density of the pathological protein aggregates as a function of time.

In certain embodiments, the method further comprises: performing image registration to a coordinate space comprising a plurality of voxels, wherein each voxel is represented as a cubic volumetric element centered at a coordinate in the coordinate space; identifying positions in x, y, z coordinates of each pathological protein aggregate in the coordinate space; measuring volumes of each pathological protein aggregate from total number of voxels occupied by each pathological protein aggregate; calculating aggregate density for each voxel, wherein the aggregate density for each voxel is determined from total number of pathological protein aggregates having centers within the same voxel; calculating total aggregate size for each voxel, wherein the total aggregate size is the total size of all the pathological protein aggregates having centers within the same voxel; calculating mean aggregate size for each voxel as the total aggregate size divided by the aggregate density for each voxel; calculating total signal intensity for each voxel from the total intensity of all the pathological protein aggregates having centers within the same voxel; and calculating mean signal intensity for each voxel as the total signal intensity divided by the aggregate density for each voxel.

In certain embodiments, modeling of the discretized distribution of the pathological protein aggregates in each neuroanatomical region is performed using a Smoluchowski network model with the following set of differential equations:

$$\frac{{dc}_{1 , j}}{dt} = - α {\sum }_{k = 1}^{V} {L}_{jk} {c}_{1 , k} - {μ}_{1 , j} {c}_{1 , j} - 2 {c}_{1 , j}^{2} ( {κ}_{j} + ξ {\sum }_{k = 2}^{N} {kc}_{2 , k} ) - {c}_{1 , j} {\sum }_{k = 2}^{N} {c}_{k , j} ,$$

$$\frac{{dc}_{2 , j}}{dt} = - {2}^{- η} α {\sum }_{k = 1}^{V} {L}_{jk} {c}_{2 , k} - {2}^{λ} {μ}_{2 , {j}^{C} 2 , j} + {c}_{1 , j}^{2} ( {κ}_{j} + ξ {\sum }_{k = 2}^{N} {kc}_{2 , k} ) - {c}_{1 , j} {c}_{2 , j} ,$$

$$\frac{{dc}_{i , j}}{dt} = - {i}^{- η} α {\sum }_{k = 1}^{V} {L}_{jk} {c}_{i , k} - {i}^{λ} {μ}_{i , j} {c}_{i , j} + {c}_{1 , j} ( {c}_{i - 1 , j} - ci , j ) , and$$

$$\frac{{dc}_{N , j}}{dt} = - {N}^{λ} {μ}_{N , {j}^{C} N , j} + {c}_{1 , j} {c}_{N - 1 , j} ,$$

wherein c_{i,j} represents the total count of pathological protein aggregates in a discretized size-bin indexed by l, in a brain region indexed by j, wherein an L matrix represents the Laplacian matrix of the weighted directed graph connecting the neuroanatomical regions of the brain, wherein η is chosen as a hyperparameter that slows the spread of large aggregates as the inverse power of the size, and wherein λ is chosen as a hyperparameter that accelerates the decay of pathological protein aggregates proportionally to the power of their size.

In certain embodiments, initial values for α and μ are fit by sweeping through a 2-dimensional-grid and selecting values that result in a lowest mean-squared error between predicted and actual counts of the pathological protein aggregates.

In certain embodiments, the initial value of μ=0, the initial value of k=0, ξ=0, and c is a one-dimensional size vector, wherein the set of differential equations simplifies to a standard network diffusion model.

In certain embodiments, the method further comprises quantifying sensitivity of the model to specific neuroanatomical pathways in the brain, wherein a Jacobian matrix is calculated by taking a partial derivative of the model's output with respect to the weight of the anatomical connection strength between two neuroanatomical regions encoded into the model, wherein an element of the Jacobian matrix represents relative contribution of the anatomical connection between the two neuroanatomical regions to the spreading of pathological protein aggregates to a specific region of the brain.

In certain embodiments, the method further comprises using the model to produce a ranking of candidate seed locations for a given pathological state c at t=T months post-injection (MPI) by a method comprising: using each of the neuroanatomical regions as separate seed locations at t=0 within the model and simulated forward in time to t=T MPI, wherein each of the simulation results for the different neuroanatomical regions are compared with an observed state c using a pairwise similarity metric, wherein the similarity metric is a correlation coefficient between total regional aggregate counts across observed and simulated states; and using the similarity metric values to sort the seed locations for the neuroanatomical regions as likely sites that lead to the observed pathological state c, wherein the ranking of candidate seed locations for the given pathological state c at t=T MPI is produced.

In certain embodiments, the method further comprises predicting the time since seeding t=T MPI for a given pathological state c by a method comprising: comparing the whole-brain distribution of aggregate sizes for state c with simulated distributions at various t using a pairwise similarity metric without taking the seed location into account, wherein the distribution of simulated aggregate sizes across the whole brain is assumed to be invariant with respect to which neuroanatomical region is used as the seed location at t=0; and calculating the mean squared error between the stimulated and observed distributions, wherein when deciding among several candidate t values, the mean squared errors are inverted and normalized to sum to 1 to provide a prediction probability for each t being the correct estimate of T for the given pathological state c.

In certain embodiments, the gene effects on regional spreading and decay of the pathological protein aggregates are determined by a method comprising: assuming that α (spreading) and μ (decay) parameters are regionally dependent, wherein the spreading from a specific neuroanatomical region is proportional to the gene density in that region; normalizing all genes to the same range so that only the regional distribution of gene expression relative to that gene's total whole-brain expression is compared, wherein α is a vector, and the product of α with the Laplacian connectivity matrix L has the effect of modifying the regional connectivity encoded into the model; and normalizing each gene vector to have a mean of 1 and a standard deviation Σ that is empirically set to preserve a correlation between predicted and observed whole-brain aggregate count, wherein the normalization is chosen so that the product has the effect of maintaining the trace of the original Laplacian connectivity matrix L. In some embodiments, derivation of the normalization for maintaining the trace of the original Laplacian connectivity matrix L comprises: assuming the vector s is sampled from a multivariate normal distribution with mean-value 1 and standard deviation Σ, wherein s˜N(1,Σ); using a definition of the matrix trace and representing s as a diagonal square matrix S, wherein the trace of the product of S and the Laplacian connectivity matrix L results in the following:

$$Tr ( SL ) = Tr ( [ \begin{matrix}{s}_{1} & ⋯ & 0 \\ ⋮ & ⋱ & ⋮ \\ 0 & ⋯ & {s}_{V}\end{matrix} ] L ) = {\sum }_{i = 1}^{V} {s}_{i} {L}_{ii} = s \cdot diag ( L ) = s \cdot l ,$$

wherein l represents the diagonal of L, and wherein the trace is equivalent to the dot product of s and l, which has an expectation value equivalent to the sum of the entries of l, which recovers the definition of the trace of L according to the following equations:

$$s \cdot l ∼ N ( 1 \cdot l , l \sum l ) \\ E [ s \cdot l ] = Tr ( L ) ,$$

wherein after each gene is encoded into the model; comparing net effects on the regional correlation between the simulated and actual data to the baseline correlation with no genes; and providing an ordered list of genes ranked by the relevance of their spatial expression map in improving the regional predictions of the model.

In certain embodiments, the cubic volumetric element has a width of 100 μm in the coordinate space.

In certain embodiments, the one or more pathological protein aggregates map to a single voxel.

In certain embodiments, the computer implemented method further comprises performing multidimensional Gaussian filtering to account for variations in image registration between different samples.

In certain embodiments, the computer implemented method further comprises segmenting the image to produce a plurality of image segments.

In certain embodiments, the locations of the pathological protein aggregates are mapped to neuroanatomical regions of the Allen Human Brain Reference Atlas. In some embodiments, mapping comprises performing image registration to transform the locations of the pathological protein aggregates to the Allen Human Brain Reference Atlas coordinate space. In some embodiments, anatomically interconnected neuroanatomical regions are identified from the Allen Connectivity Atlas.

In certain embodiments, the neuroanatomical regions are selected from an Anterior amygdalar area, Anterior cingulate area, dorsal part, Anterior cingulate area, ventral part, Nucleus accumbens, Anterodorsal nucleus, Anterior hypothalamic nucleus, Agranular insular area, dorsal part, Agranular insular area, posterior part, Agranular insular area, ventral part, Nucleus ambiguous, Anteromedial nucleus, dorsal part, Anteromedial nucleus, ventral part, Ansiform lobule, Accessory olfactory bulb, Anterior olfactory nucleus, Anterior pretectal nucleus, Arcuate hypothalamic nucleus, Dorsal auditory area, Primary auditory area, Ventral auditory area, Anteroventral nucleus of thalamus, Basolateral amygdalar nucleus, Basomedial amygdalar nucleus, Bed nuclei of the stria terminalis, Field CA1, Field CA2, Field CA3, Central amygdalar nucleus, Central lobule, Central lateral nucleus of the thalamus, Claustrum, Central linear nucleus raphe, Central medial nucleus of the thalamus, Cortical amygdalar area, anterior part, Cortical amygdalar area, posterior part, Caudoputamen, Superior central nucleus raphe, Culmen, Cuneiform nucleus, Dorsal cochlear nucleus, Dentate gyrus, Dorsomedial nucleus of the hypothalamus, Dentate nucleus, Dorsal peduncular area, Dorsal nucleus raphe, Ectorhinal area, Entorhinal area, lateral part, Entorhinal area, medial part, dorsal zone, Endopiriform nucleus, dorsal part, Endopiriform nucleus, ventral part, Flocculus, Fastigial nucleus, Frontal pole, cerebral cortex, Fundus of striatum, Globus pallidus, external segment, Globus pallidus, internal segment, Gigantocellular reticular nucleus, Gustatory areas, Intercalated amygdalar nucleus, Inferior colliculus, central nucleus, Inferior colliculus, dorsal nucleus, Inferior colliculus, external nucleus, Infralimbic area, Intermediodorsal nucleus of the thalamus, Inferior olivary complex, Interposed nucleus, Interpeduncular nucleus, Intermediate reticular nucleus, Lateral amygdalar nucleus, Lateral vestibular nucleus, Lateral dorsal nucleus of thalamus, Dorsal part of the lateral geniculate complex, Ventral part of the lateral geniculate complex, Lateral habenula, Lateral hypothalamic area, Lateral posterior nucleus of the thalamus, Lateral preoptic area, Lateral reticular nucleus, Lateral septal nucleus, caudal (caudodorsal) part, Lateral septal nucleus, rostral (rostroventral) part, Lateral septal nucleus, ventral part, Magnocellular nucleus, Magnocellular reticular nucleus, Mediodorsal nucleus of thalamus, Medullary reticular nucleus, dorsal part, Medullary reticular nucleus, ventral part, Medial amygdalar nucleus, Median preoptic nucleus, Medial geniculate complex, dorsal part, Medial geniculate complex, medial part, Medial geniculate complex, ventral part, Medial habenula, Medial mammillary nucleus, Main olfactory bulb, Primary motor area, Secondary motor area, Medial preoptic nucleus, Medial preoptic area, Medial pretectal area, Midbrain reticular nucleus, Medial septal nucleus, Medial vestibular nucleus Diagonal band nucleus, Nucleus incertus, Nucleus of the lateral lemniscus, Nucleus of the lateral olfactory tract, Nodulus (X), Nucleus of the optic tract, Nucleus of the posterior commissure, Nucleus of the solitary tract, Orbital area, lateral part, Orbital area, medial part, Orbital area, ventrolateral part, Olfactory tubercle, Posterior amygdalar nucleus, Piriform-amygdalar area, Periaqueductal gray, Parasubiculum, Parvicellular reticular nucleus, Parabrachial nucleus, Pontine central gray, Perirhinal area, Parafascicular nucleus, Paraflocculus, Pontine gray, Paragigantocellular reticular nucleus, dorsal part, Paragigantocellular reticular nucleus, lateral part, Posterior hypothalamic nucleus, Piriform area, Prelimbic area, Dorsal premammillary nucleus, Posterior complex of the thalamus, Posterior limiting nucleus of the thalamus, Postsubiculum, Peripeduncular nucleus, Pedunculopontine nucleus, Presubiculum, Paramedian lobule, Pontine reticular nucleus, caudal part, Pontine reticular nucleus, Nucleus prepositus, Principal sensory nucleus of the trigeminal, Parataenial nucleus, Posterior parietal association areas, Paraventricular hypothalamic nucleus, Paraventricular nucleus of the thalamus, Periventricular hypothalamic nucleus, posterior part, Periventricular hypothalamic nucleus, preoptic part, Pyramus (VIII), Retrochiasmatic area, Nucleus of reuniens, Rhomboid nucleus, Nucleus raphe magnus, Red nucleus, Midbrain reticular nucleus, retrorubral area, Retrosplenial area, lateral agranular part, Retrosplenial area, dorsal part, Retrosplenial area, ventral part, Reticular nucleus of the thalamus, Subparaventricular zone, Superior colliculus, motor related, Superior colliculus, sensory related, Septofimbrial nucleus, Substantia innominate, Simple lobule, Submedial nucleus of the thalamus, Substantia nigra, compact part, Substantia nigra, reticular part, Superior olivary complex, Subparafascicular area, Subparafascicular nucleus, magnocellular part, Subparafascicular nucleus, parvicellular part, Spinal vestibular nucleus, Spinal nucleus of the trigeminal, caudal part, Spinal nucleus of the trigeminal, interpolar part, Spinal nucleus of the trigeminal, oral part, Primary somatosensory area, barrel field, Primary somatosensory area, lower limb, Primary somatosensory area, mouth, Primary somatosensory area, nose, Primary somatosensory area, trunk, Primary somatosensory area, upper limb, Supplemental somatosensory area, Subthalamic nucleus, Subiculum, Supramammillary nucleus, Supratrigeminal nucleus, Superior vestibular nucleus, Temporal association areas, Postpiriform transition area, Tegmental reticular nucleus, Triangular nucleus of septum, Taenia tecta, Tuberal nucleus, Motor nucleus of trigeminal, Ventral anterior-lateral complex of the thalamus, Ventral cochlear nucleus, Facial motor nucleus, Visceral area, Anterolateral visual area, Anteromedial visual area, Lateral visual area, Primary visual area, Posterolateral visual area, posteromedial visual area, Ventral medial nucleus of the thalamus, Ventromedial hypothalamic nucleus, Ventral posterolateral nucleus of the thalamus, Ventral posteromedial nucleus of the thalamus, Ventral posteromedial nucleus of the thalamus, parvicellular part, Ventral tegmental area, and Hypoglossal nucleus.

In certain embodiments, the computer implemented method further comprises predicting where pathological protein aggregates originated in the brain of the subject.

In certain embodiments, the machine learning algorithm uses an artificial neural network.

In certain embodiments, the machine learning algorithm uses a deep learning algorithm.

In certain embodiments, the deep learning algorithm uses a convolutional neural network, a deep neural network, a recurrent neural network, a deep residual neural network, a long short-term memory network, a deep belief network, a multilayer perceptron, or deep reinforcement learning.

In certain embodiments, the machine learning algorithm is supervised, semi-supervised, or unsupervised.

In certain embodiments, the subject is a human subject.

In certain embodiments, modeling spreading, aggregation, and decay of the pathological protein aggregates in the brain of the human subject uses a simulation generated based on measuring spreading, aggregation, and decay of the pathological protein aggregates in a non-human animal model, wherein pathology measured in the non-human animal model experimentally is used to design simulation parameters to predict the past locations, the present locations, and the future locations of the pathological protein aggregates in the human subject.

In certain embodiments, the simulation further uses brain anatomy or brain function measured from the non-human animal to predict the past locations, the present locations, and the future locations of the pathological protein aggregates in the human subject.

In certain embodiments, the non-human animal is a mammal. In some embodiments, the mammal is a rodent or a primate. In some embodiments, the rodent is a mouse.

In certain embodiments, the simulation further uses brain anatomy or brain function measured from other human subjects to design simulation parameters to predict the past locations, the present locations, and the future locations of the pathological protein aggregates in the human subject.

In certain embodiments, the computer implemented method further comprises: receiving a second image of the brain, wherein the second image is taken after said neurostimulation is applied to the brain; repeating steps (b)-(o) using the second image; and displaying changes in total aggregate size for each voxel, volume of each pathological protein aggregate for each voxel, and aggregate density for each voxel in the image taken after said neurostimulation is applied to the brain of the subject compared to the image taken before said neurostimulation is applied to the brain of the subject.

In certain embodiments, the computer implemented method further comprises: receiving a second image of the brain, wherein the second image is taken after said neurostimulation is applied to the brain; repeating steps (b)-(o) using the second image; modulating one or more programmed neurostimulation parameters based on any changes in the past locations, present locations, or future locations where the pathological protein aggregates are predicted to occur; and instructing a neurostimulation device to apply modulated neurostimulation to the brain of the subject in order to treat the neurological or neurodegenerative disease in the subject.

In certain embodiments, the computer implemented method further comprises storing a user profile for the subject comprising information regarding the programmed neurostimulation parameters used to apply neurostimulation to the brain of the subject to treat the neurological or neurodegenerative disease based on where the computer implemented method predicts pathological protein aggregates are present or will develop at a future time.

In another aspect, a non-transitory computer-readable medium comprising program instructions that, when executed by a processor in a computer, causes the processor to perform a method, described herein, is provided.

In another aspect, a kit comprising the non-transitory computer-readable medium and instructions for treating a neurological or a neurodegenerative disease in a subject with neurostimulation is provided.

In another aspect, a method for treating a neurological or neurodegenerative disease in a subject is provided, the method comprising: imaging pathological protein aggregates in the brain of the subject; using a computer implemented method, described herein, to predict where pathological protein aggregates will develop based on locations of the pathological protein aggregates that are detected in the brain of the subject by said imaging; and applying neurostimulation at locations in the brain where the pathological protein aggregates are detected in the brain of the subject by said imaging and at locations where the computer implemented method predicts pathological protein aggregates will develop.

In certain embodiments, imaging is performed using computed tomography (CT), single photon emission computed tomography (SPECT), magnetic resonance imaging, functional magnetic resonance imaging, optogenetic functional magnetic resonance imaging, or positron emission tomography (PET).

In certain embodiments, the method further comprises adjusting stimulation frequency and pulse width of the neurostimulation to target specific neuronal cell-types or circuits within the brain at the locations in the brain where the computer implemented method predicts the pathological protein aggregates are present or will develop at the future time.

In certain embodiments, the neurological or neurodegenerative disease is a synucleinopathy such as, but not limited to, Parkinson's disease, dementia with Lewy bodies, multiple system atrophy, neuroaxonal dystrophy Shy-Drager syndrome, striatonigral degeneration, or olivopontocerebellar atrophy.

In certain embodiments, the neurological or neurodegenerative disease is Alzheimer's disease, amyotrophic lateral sclerosis, or frontotemporal dementia.

In certain embodiments, the pathological protein aggregates comprise alpha-synuclein aggregates.

In certain embodiments, applying neurostimulation comprises applying neurostimulation using an electrode.

In certain embodiments, the electrode is a depth electrode or a surface electrode.

In certain embodiments, the electrode is a non-brain penetrating surface electrode array or a brain-penetrating electrode array.

In certain embodiments, applying neurostimulation comprises applying deep brain stimulation, transcranial magnetic stimulation, or transcranial electrical stimulation.

In certain embodiments, applying neurostimulation comprises applying neurostimulation optogenetically.

In certain embodiments, neurostimulation is applied optogenetically by a method comprising: introducing a recombinant polynucleotide encoding a light-responsive ion channel into a neuron at the location in the brain where the computer implemented method predicts pathological protein aggregates are present or will develop at a future time, wherein the light-responsive ion channel is expressed in the neuron; and illuminating the light-responsive ion channel with light at a wavelength that activates the light-responsive ion channel, wherein conduction of ions by the light-responsive ion channel in response to absorption of light results in hyperpolarization or depolarization of the neuron.

In certain embodiments, the light-responsive ion channel is a light-responsive anion-conducting opsin or a light-responsive proton conductance regulator.

In certain embodiments, the light-responsive anion-conducting opsin conducts chloride ions (Cl⁻).

In certain embodiments, the anion-conduction opsin is an anion-conducting channelrhodopsin or halorhodopsin.

In certain embodiments, the halorhodopsin is a Natronomonas pharaonis halorhodopsin (NpHR), enhanced NpHR (eNpHR) 1.0, eNpHR 2.0, or eNpHR 3.0.

In certain embodiments, the anion-conducting channelrhodopsin is iC1C2, SwiChR, SwiChR++, or iC++.

In certain embodiments, the light-responsive proton conductance regulator is a bacteriorhodopsin or an archaerhodopsin.

In certain embodiments, the light-responsive proton conductance regulator is Arch from Halorubrum sodomense, ArchT from Halorubrum sp., TP009 from Leptosphaeria maculans, or Mac from Leptosphaeria maculans.

In certain embodiments, the light-responsive ion channel is a light-responsive cation-conducting opsin.

In certain embodiments, the light-responsive cation-conducting opsin conducts calcium cations (Ca²⁺).

In certain embodiments, the light-responsive cation-conducting opsin is a light-responsive cation-conducting channelrhodopsin.

In certain embodiments, the light-responsive cation-conducting channelrhodopsin is a Chlamydomonas reinhardtii channelrhodopsin or a Volvox carteri channelrhodopsin.

In certain embodiments, the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

In certain embodiments, the polynucleotide encoding the light-responsive ion channel is provided by a viral vector.

In certain embodiments, the viral vector is a lentiviral vector or an adeno-associated viral (AAV) vector.

In certain embodiments, the viral vector is stereotactically injected into the brain at the location where the computer implemented method predicts pathological protein aggregates are present or will develop at a future time.

In certain embodiments, the vector further comprises a neuron-specific promoter operably linked to the polynucleotide encoding the light-responsive ion channel.

In certain embodiments, expression of the light-responsive ion channel is inducible.

In certain embodiments, illuminating the light-responsive ion channel comprises delivering light from a light source to the light-responsive ion channel using a fiber-optic-based optical neural interface.

In certain embodiments, the light source is a solid-state diode laser.

In certain embodiments, applying neurostimulation comprises applying neurostimulation to a motor cortex region or a subcortical region of the brain.

In certain embodiments, multiple cycles of the neurostimulation are performed.

In certain embodiments, the method further comprises assessing effectiveness of the treatment of the neurological or neurodegenerative disease in the subject. In some embodiments, said assessing comprises imaging the brain of the subject to measure sizes and identify locations of the pathological protein aggregates after said neurostimulation. In some embodiments, said assessing comprises measuring brain function of the subject after said neurostimulation. For example, brain function may be measured by performing electroencephalography (EEG), stereoelectroencephalography (sEEG), electrocorticography (ECoG), magnetoencephalography (MEG), single photon emission computed tomography (SPECT), functional magnetic resonance imaging (fMRI), optogenetic functional magnetic resonance imaging, or positron emission tomography (PET).

In certain embodiments, the method further comprises modulating one or more programmed neurostimulation parameters to improve the brain function.

In another aspect, a system for treating a neurological or neurodegenerative disease in a subject is provided, the system comprising: a neurostimulation device; and a processor programmed according to a computer implemented method, described herein, to instruct the neurostimulation device to deliver neurostimulation to the brain of the subject in a manner effective to treat the neurological or neurodegenerative disease in the subject, wherein neurostimulation is applied to the brain at predicted present locations of the pathological protein aggregates, at predicted future locations of the pathological protein aggregates, or at predicted past locations of the pathological protein aggregates, or a combination thereof.

In certain embodiments, the neurostimulation device comprises an electrode. In some embodiments, the electrode is a depth electrode or a surface electrode. In some embodiments, the electrode is a non-brain penetrating surface electrode array or a brain-penetrating electrode array.

In certain embodiments, the neurostimulation device performs deep brain stimulation, transcranial magnetic stimulation, or transcranial electrical stimulation.

In certain embodiments, the system further comprises a user interface comprising an input electronically coupled to the processor for instructing the neurostimulation device to apply neurostimulation to the brain of the subject to treat the neurological or neurodegenerative disease in the subject.

In certain embodiments, the user interface is password protected and is operable by a health care practitioner.

In certain embodiments, the system further comprises a display. In some embodiments, the display displays an image of the brain of the subject showing the predicted present locations, past locations, or future locations of the pathological protein aggregates, as determined by the computer implemented method. In some embodiments, the display displays information regarding the coordinates of each pathological protein aggregate, the volume of each pathological protein aggregate; the aggregate density for each voxel, the total aggregate size for each voxel, the mean aggregate size for each voxel, the total signal intensity for each voxel, the mean signal intensity for each voxel, or the mapping of the positions of the pathological protein aggregates to neuroanatomical regions, or any combination thereof. In some embodiments, the display displays information regarding the distribution of gene effects on regional spreading and decay of the pathological protein aggregates. In some embodiments, the display displays information regarding the predicted changes in regional density of the pathological protein aggregates as a function of time determined by the modeling of the spreading, aggregation, decay, and the spatial gene expression of the pathological protein aggregates throughout the neuroanatomical regions. In some embodiments, the display displays information regarding the predicted past locations, present locations, and future locations of the pathological protein aggregates based on the modeling.

In certain embodiments, the system is for use in treating a synucleinopathy such as, but not limited to, Parkinson's disease, dementia with Lewy bodies, multiple system atrophy, neuroaxonal dystrophy Shy-Drager syndrome, striatonigral degeneration, or olivopontocerebellar atrophy.

In certain embodiments, the system is for use in treating Alzheimer's disease, amyotrophic lateral sclerosis, or frontotemporal dementia.

### BRIEF DESCRIPTION OF THE DRAWINGS

FIGS. 1A-1G. Tissue clearing and light sheet fluorescence microscopy capture changes in whole-brain pathological at various time points post-seeding. (FIG. 1A) α-syn PFFs were unilaterally injected into the striatum of mice, and cohorts of mice were perfused at various timepoints ranging from 2 weeks to 18 months post-injection (MPI). Each extracted mouse brain was processed for fluorescent immunolabeling of α-syn pathology and whole-brain clearing using the iDISCO+ protocol. Brains were three-dimensionally imaged by light-sheet fluorescent microscopy to visualize both the antibody fluorescence and tissue autofluorescence for anatomical mapping. (FIG. 11B) Axial projections of autofluorescence from an imaged mouse brain (left) and α-syn pSer129 immunolabeled pathology (right). (FIG. 1C) A quantitative pipeline registers the autofluorescence to an anatomical atlas, and a trained classifier (FIG. 1D) segments and α-syn pathology. (FIG. 1E) Both whole-brain spreading and subsequent decline of pathology are observed in glass-brain reconstructions of representative samples at each timepoint, with each aggregate color-coded by Allen Reference Atlas region. (FIG. 1F) Total α-syn inclusion count versus time post-injection quantifies this trend of spreading followed by decay. (FIG. 1G) Normalized density distributions of mean aggregate size at each voxel across the various timepoints depict the general increase in aggregate volume during initial apparent prion-like spread, followed by a decrease as large aggregates diminish. Data are represented as mean±SD. Isoctx—isocortex, TH—thalamus, HY—hypothalamus, MB—midbrain, HB—hindbrain, CB—cerebellum. See also FIGS. 6, 8, and 9.

FIGS. 2A-2C. Statistical analysis at both regional and voxel level demonstrates biphasic spreading and decay interleaved between cortical and subcortical areas. (FIG. 2A) The computational pipeline used for processing each brain sample consists of registration to a reference atlas, segmentation of three-dimensional aggregate volume, and using these two to map each aggregate to a neuroanatomical region or voxel in a shared coordinate space in the Allen Reference Atlas (ARA). This allows for statistical comparisons between longitudinal groups, at both the regional and brain-voxel level. (FIG. 2B) Voxel-level statistics using heatmaps from pairs of time points facilitates the discovery of voxel clusters with a statistically significant (p<0.05) vulnerability to initial pathological spread, and separately accumulation of mean aggregate volume. (FIG. 2C) Grouping into ARA regions before statistical testing yields similar results. Isoctx—isocortex, OLF—olfactory areas, HPF—hippocampal formation, CTX sp—cortical subplate, CNU—caudate nucleus, TH—thalamus, HY—hypothalamus, MB—midbrain, HB—hindbrain, CB—cerebellum. See also FIG. 10 and Table S1.

FIGS. 3A-3E. Computational model describes spreading, aggregation, and decay. (FIG. 3A) The computational model describes the spreading of aggregates throughout the nodes of a directed graph, which relies on anatomical connectivity estimates from the Allen Connectivity Atlas. Each node represents an atlas region, with each edge representing the anatomical neuronal connectivity between the two regions. Thicker lines represent higher anatomical connections. (FIG. 3B) To model the interactions between aggregates of various sizes in the model, each aggregate's volume is discretized into one of several size bins which are tracked as separate model variables in each region. Discrete-sized particles within each region can accumulate, with volumes combining additively. (FIG. 3C) Fitted model accurately simulates both the longitudinal whole-brain counts of each discretized aggregate size (r=0.98), and the regional counts (r=0.72) for each size. (FIG. 3D) The raw time-series output from the computational model demonstrates the model's ability to capture the dynamics of each discretized aggregate size. Black lines represent the model prediction of total aggregates of a given size, and gray lines represent the actual observed count. (FIG. 3E) Jacobian calculation between adjacent time points quantifies the model's sensitivity to specific anatomical connections. The top 10% Jacobian elements for each pair of timepoints are displayed. Isoctx—isocortex, TH—thalamus, HY—hypothalamus, MB—midbrain, HB—hindbrain, CB—cerebellum. See also FIG. 11.

FIGS. 4A-4E. Seeding of α-syn fibrils in different brain regions results in consistent volumetric distributions of aggregate formation yet distinct spreading patterns by region, both of which are predicted by the computational model fitted to the striatal dataset. (FIG. 4A) α-syn PFFs are injected into new seed locations, with independent cohorts for the main olfactory bulb (MOB), substantia nigra (SN), and dentate gyrus (DG). Mice are perfused at 0.5 MPI, 2 MPI, and 4 MPI. (FIG. 4B) Distributions of aggregate sizes for the various seed locations demonstrate consistencies across various timepoints. 0.5 MPI samples consistently contained a higher number of small aggregates, while this distribution shifts towards larger aggregates for later time points. Data are represented as mean±SD. However, (FIG. 4C) voxel-level and (FIG. 4D) regional statistics from 0.5 MPI to 4 MPI demonstrate that different seed locations result in distinct downstream spreading patterns. (FIG. 4E) The correlation of each in vivo seed location with the model output from in silico seeding of all 424 regions in the ARA. Both ipsilateral and contralateral results for MOB, SN, and DG are labeled. In all cases, the model can accurately differentiate between unseen datasets with various distinct α-syn PFF seed locations. For each additional seeding site, side-by-side comparisons of the actual and simulated histograms of aggregate volume, and confusion matrices between the actual and simulated states, demonstrate that the model can use the histogram of discretized aggregate sizes to predict the progression (MPI) since initial seeding. See also FIGS. 12 and 13.

FIGS. 5A-5E. Integrating spatial transcriptomics data into computational model reveals genes associated with spreading across seed locations. (FIG. 5A) Encoding region-specific gene densities from the Allen ISH database into the model allows for comparisons of each gene's association with the spreading and decay parameters in improving predictive power. (FIG. 5B) Joint heatmap of the spreading and decay gene rankings depict clustering of genes that are relevant for either spreading or decay. Genes implicated in Parkinson's Disease and synucleinopathies are additionally labeled. (FIG. 5C) Histograms of genes for each parameter that improved model performance. Genes are grouped by cell type with highest transcription levels of that gene, taken from the Allen Atlas. (FIG. 5D) Simulation results from all genes in the Allen ISH database tested separately for the various PFF seed locations. The regional correlation between each gene's simulated output and the entire timeseries for the seed location is reported, with the shared gene ordering on the x-axis determined by the striatum's ranked genes in ascending order. (FIG. 5E) Encoding the highly ranked genes from the striatum dataset consistently improved the predictive power of the model for other seed regions, as measured by the correlation coefficient, while the bottom percentile genes consistently decreased the predictive power. Data are represented as mean±SD.

FIG. 6. Three-dimensional acquisitions from whole-brain tissue clearing and light sheet microscopy provide neuroanatomical contrast and readout of whole-brain α-syn pathology, Related to FIG. 1. Sagittal and axial projections of autofluorescence. Sagittal and axial projections of pSer129 labeling.

FIGS. 7A-7D. Serial histological sectioning detects α-syn pathology in similar regions as whole-brain methods but is insufficient for longitudinal or regional comparisons, Related to STAR Methods. (FIG. 7A) Traditional serial histology shows staining patterns across striatum and layers of several cortical regions. (FIG. 7B) Distributions along AP axis of histological coronal sections taken from animals 2 and 6 MPI. (FIG. 7C) Comparison between regional aggregate count between iDISCO acquisitions (top; 2,6 MPI) and extrapolated results from traditionally immunolabeled sections (bottom; 2,6 MPI). (FIG. 7D) Results show that serial histology estimates capture neither absolute number of aggregates nor relative numbers between regions. Data are represented as mean±SD. Isoctx—isocortex, OLF—olfactory areas, HPF—hippocampal formation, CTX sp—cortical subplate, CNU—caudate nucleus, TH—thalamus, HY—hypothalamus, MB—midbrain, HB—hindbrain, CB—cerebellum.

FIGS. 8A-8C. Registration pipeline accurately places three-dimensional acquisitions into the shared ARA coordinate space, Related to FIG. 1. (FIG. 8A) We optimized registration quality using a mutual-information similarity metric which converges to a similar range of values across the samples. (FIG. 8B) We calculated the linear expansion of the cleared brains using the singular values of the affine transformation matrix used in the registration process. (FIG. 8C) The green contours of the atlas were overlaid on the brain's autofluorescence channel in both the imaging plane (sagittal) and resliced planes (coronal, axial) to depict the registration quality. Data are represented as mean±SD.

FIGS. 9A-9B. Segmentation pipeline performs accurate detection of pathological aggregates, Related to FIG. 1. (FIG. 9A) Overview of machine learning classifier (random forest) that is trained on three-dimensional structural features and produces a probability of each voxel as foreground (pathology) or background. The ROC and PRC curves quantify segmentation performance on a test dataset. (FIG. 9B) Raw extracts of pSer129 labeling alongside corresponding segmented maps.

FIG. 10. Heatmap quantification for each time point after CP seeding depicts spreading and decline. Montages of the mean voxel-level density map for all acquired time points after PFF seeding in the striatum.

FIGS. 11A-11E. Computational model parameters were optimized then evaluated on the entire dataset. (FIG. 11A) The anatomical connectivity matrix describing both ipsilateral and contralateral connection strengths between 212 regions (taken from the Allen Connectivity Atlas), and (FIG. 11B) sections from the ARA atlas used for grouping aggregates into neuroanatomical regions. (FIG. 11C) Various versions of Laplacian matrix of the directed weighted graph representing regional connectivity were tested, including both anterograde and retrograde anatomical connectivity matrices from the Allen Connectivity Atlas, and a matrix weighted by the Euclidean distances between regions. (FIG. 11D) Tree depicting the hierarchy of brain regions in the ARA, with the regions used for the model highlighted in red. (FIG. 11E) The number of size bins used for the discretization of sizes was also swept through. Increasing this hyperparameter improves the model's performance at a diminishing rate. In order to lower the dimensionality of the model's output and stay within computational limitations, we chose a discretization of 7 equally spaced sizes for all simulations. Similarly, increasing the number of regions used in the model only improved the model's performance. Isoctx—isocortex, TH—thalamus, HY—hypothalamus, MB—midbrain, HB—hindbrain, CB—cerebellum.

FIG. 12. Heatmap quantification of additional seed locations reveals distinct early spreading patterns. Montages of the mean voxel-level density map for all acquired time points after PFF seeding in the main olfactory bulb (MOB), substantia nigra (SN), and dentate gyrus (DG).

FIGS. 13A-13B. Model generalizes in predicting whole-brain and regional spreading patterns from alternate α-syn seeding sites. (FIG. 13A) For different seeding sites, the model trained on the striatal dataset can accurately predict counts of α-syn aggregate counts longitudinally across the whole brain, across the various discretized sizes, and across neuroanatomical regions. (FIG. 13B) Statistical tests across neuroanatomical regions detect distinct changes in aggregate count or mean-size for each seeding site. Isoctx—isocortex, OLF—olfactory areas, HPF—hippocampal formation, CTX sp—cortical subplate, CNU—caudate nucleus, TH—thalamus, HY—hypothalamus, MB—midbrain, HB—hindbrain, CB—cerebellum.

FIGS. 14A-14C. Quantification of alpha-synuclein pathology following whole brain immunolabeling and clearing informs candidate regions to target for neuromodulation. (FIG. 14A) Following injection of α-synuclein into the striatum, whole brain tissue-clearing and imaging captures pathological spreading from the striatum to many remote brain regions at 2 WPI. (FIG. 14B) A quantification pipeline performs detection of each aggregate while aligning to the ARA. (FIG. 14C) Regional grouping of alpha synuclein spread and comparisons across multiple subjects provides a candidate list of target regions for stimulation, from which the motor areas consistently demonstrate high pathological levels.

FIGS. 15A-15B. Repeated optogenetic stimulation of motor areas following injection of alpha-synuclein PFFs into the striatum alters whole brain pathology. (FIG. 15A) Schematic depicting the experimental paradigm for the injection of α-synuclein PFFs into the striatum and implantation of an optical fiber into the Secondary Motor Area (MOs), followed by daily optogenetic stimulations for two weeks. Each daily stimulation consisted of ten 1-minute stimulation periods with 1-minute rest between each period. (FIG. 15B) Maximum intensity projections (MIP) of cleared and labeled brains, alongside zoomed-in cortical sections from the ipsilateral hemisphere, depict the decrease in pathological aggregation from the control to stimulated group.

FIGS. 16A-16B. Whole brain statistical analysis validates that optogenetic stimulation modulates α-synuclein inclusion count at both the individual voxel and neuroanatomical regional levels. (FIG. 16A) Statistical comparisons at the voxel-level between the treatment and control group reveal that the pathological aggregate count is significantly decreased in many ipsilateral subcortical clusters and near the site of stimulation, while the aggregate count significantly increases in many contralateral cortical clusters. (FIG. 16B) The optogenetic stimulation effect quantified across neuroanatomical regions, either by counting the number of significant voxels per region and hemisphere, or by calculating the total aggregate count by region and performing statistical comparisons across treatment and control groups, show results consistent with voxel-level quantifications.

FIGS. 17A-17C. Whole brain functional activity measured with optogenetic fMRI during optogenetic stimulation is predictive of downstream pathological changes. (FIG. 17A) Brain-wide BOLD fMRI was measured during optogenetic stimulation of the Secondary Motor Area (Layer V). (FIG. 17B) BOLD activation map overlaid with statistical changes in pathology as measured by iDISCO show high colocalization between positive BOLD and decrease in aggregate count, while negative BOLD colocalizes with increases in aggregate count. (FIG. 17C) Statistical activation maps depict positive functional activity at the site of stimulation and subcortical regions, and negative activity in the contralateral cortex.

FIG. 18. Cleared and immunolabeled brains allow for capturing of whole brain pathological state. Maximum intensity projections of cleared brains imaged with light-sheet fluorescent microscopy depict whole brain spreading of pathological α-synuclein after injection into the striatum. Bottom ends of the white lines indicate site of stimulation in the Secondary Motor Area (Layer V) for stimulated subjects. Tip of the inverted triangles indicate injection site for alpha-synuclein PFFs in the striatum for both control and stimulated subjects.

FIG. 19. Optogenetic stimulation power was selected by finding the minimum optogenetic laser power required to produce consistent rotational behavior. On each stimulation day for a given subject, the power was ramped up until consistent rotational behavior was observed.

FIG. 20. Comparison of pathology between control and stimulated mice across two separate cohorts of subjects. Statistical maps comparing α-synuclein aggregate counts at the voxel-level for two different cohorts of animals that went through two separate batches of iDISCO processing. Each cohort consisted of separate control and stimulated groups, all imaged at 2 weeks post-injection (WPI). A consistent stimulation effect, which consists of primarily decreased ipsilateral aggregation and increased contralateral aggregation, is apparent in both cohorts.

FIG. 21. Wild type mice with no ChR2 expression exhibit no change in whole brain pathology following 2 weeks of sham stimulation. Statistical comparisons of whole brain pathology between control mice and sham stimulation mice at 2 weeks post-injection. Wild type mice had no ChR2 expression but still received daily laser power. Voxel-based statistical maps between control and sham mice show little to no change in aggregate count.

FIG. 22. Individual ofMRI activation maps for each subject. Rows represent a subject used for optogenetic fMRI, and single columns at each row represent fMRI activity maps for a six-minute acquisition. Each image is a thresholded statistical map (p<0.01, corrected) depicting both positive and negative activity based on the z-scores from a generalized linear model fit to the timeseries for a voxel. ofMRI maps are highly consistent within and across all subjects.

FIGS. 23A-23F. Optogenetic functional magnetic resonance imaging bridges scale. (FIG. 23A) Optogenetics enable cell-type-specific stimulations, such as the selective targeting of D1- or D2-MSNs in the striatum. (FIG. 23B) With selective stimulations of D1- or D2-MSNs in the striatum, mouses show contraversive or ipsiversive rotations, respectively (D1-MSN stim: n=12 animals, mean±SEM, ***p<0.001, two-tailed paired t test; D2-MSN stim: n=11 animals, mean±SEM, *p<0.05, **p<0.005, two-tailed paired t test). (FIG. 23C) Optogenetic fMRI technology combines optogenetic stimulations with fMRI readout. (FIG. 23D) ofMRI with selective stimulation of D1- and D2-MSN results in distinct brain-wide activities that are associated with distinct behavior (n=12 animals). Group-wise phase maps, which were thresholded to only active voxels within the brain, depict the heterogeneity in the temporal dynamics of the evoked responses. (FIG. 23E) Time series of any region can be extracted from the four-dimensional fMRI data. (FIG. 23F) Electrophysiology recordings mirror fMRI response in polarity of neural activity change. This figure is based on Lee et al. (2).

FIGS. 24A-24G. Computational modeling of ofMRI data reveals brain-wide functional interaction dynamics. (FIG. 24A) Cortico-basal-ganglia-thalamus network involves a large number of network nodes across the brain. (FIG. 24B) Direct and indirect pathways' anatomical connectivity involve large number of common anatomical regions with distinct cell types in caudate putamen (CPu). (FIG. 24C) Anatomical connections were used as a priori generative network model. In addition to direct and indirect pathways shown in (FIG. 24B), other established anatomical connections, e.g., hyper-direct pathway, were also included. (FIG. 24D) DCM generated fMRI time series closely match experimental ofMRI time series. (FIG. 24E) DCM utilized ofMRI data to estimate the causal influence (effective connectivity) among regions of interest during D1- and D2-MSN stimulations, respectively. (FIGS. 24F and 24G) The graph and matrix representations of effective connectivity network for (FIG. 24F) D1-MSN, and (FIG. 24G) D2-MSN stimulations, respectively. Significant and close-to-significant represent parameters with p<0.05 and 0.05 s p<0.10, respectively (one-sample t test, multiple comparison correction across connections with FDR p<0.10). CPu: caudate putamen; GPe: external globus pallidus; GPi: internal globus pallidus; STN: subthalamic nucleus; SNr: substantia nigra; THL: thalamus; CTX: cortex. This figure is based on Bernal-casas et al. (4).

FIGS. 25A-25D. Brain circuit function modeling at the single-cell-spiking level can be made possible through a multi-scale approach. (FIG. 25A) Locations of optogenetic stimulation and in vivo extracellular recordings for the cortico-basal-ganglia-thalamus network study are schematically illustrated. (FIG. 25B) Single-cell-spiking level modeling with ofMRI and single-unit recordings data is exemplified. A large-scale model built with ofMRI data is expanded to single-neuron level biophysical model. Each ROI consists of many simulated single neurons. The model is validated by directly comparing simulated spiking trains with experimental data. (FIG. 25C) Models can allow experimental single-unit recording data and simulated data to be directly compared. (FIG. 25D) Models should be designed so that the spike rates of all ROIs simulated by the single-cell spiking level biophysical model statistically match experimental data. ofMRI combined with biophysics modeling can enable successful reproduction of the single-cell-spiking level dynamics induced by cell type specific optogenetic stimulations such as D1- and D2-MSN stimulations.

FIGS. 26A-26F. To understand pathology function interaction, whole-brain pathology dynamics can be modeled alongside ofMRI. (FIG. 26A) α-synuclein PFFs injected into seed locations induce pathology at various time points post-injection, which can then be captured by DISCO tissue clearing and light-sheet fluorescent microscopy (LSFM). Machine-learning based, automatic segmentation and registration techniques can streamline the quantification of each pathological marker within the Allen Reference Atlas (ARA). (FIG. 26B) Comparisons of averaged heatmaps across cohorts can depict whole-brain pathology changes over many months after the injection. (FIG. 26C) Modeling of longitudinal data based on whole-brain anatomical connectivity can capture regional differences in pathology. (FIG. 26D) Reweighting the connectivity matrix can allow for encoding of genetic contribution within a model. (FIG. 26E) Whole-brain colocalization analysis between optogenetic-stimulation induced alpha-synuclein pathology change and optogenetic fMRI activity can reveal pathology function relationship. In this example, positive activity is colocalized with decreases in pathology, while negative activity is highly colocalized with increases in pathology. (FIG. 26F) Montages of the modulated alpha-synuclein pathology and ofMRI brain activity maps show high degree of colocalization with opposite polarity.

FIG. 27. Schematic showing clinical use of the technology: Pathology is imaged in a subject. The technology estimates dynamics of the pathology, including simulation of pathological spread throughout the brain. The technology is used to optimize neuromodulation therapy for treating pathology.

FIG. 28. Schematic of technological approaches.

### DETAILED DESCRIPTION OF THE INVENTION

Methods, systems, and devices, including computer programs encoded on a computer storage medium are provided for optimizing neurostimulation therapy for treatment of neurological and neurodegenerative diseases. In particular, an algorithm is used to provide a predicted regional pathological density map of neuropathology and predict locations of future spreading. Neurostimulation therapy parameters including the location, strength, and frequency of neurostimulation can be adjusted accordingly to treat neuropathology and reduce spreading.

Before the present methods, systems, and devices, including computer programs encoded on a computer storage medium are described, it is to be understood that this invention is not limited to particular methods, systems, devices, or computer programs encoded on a computer storage medium described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a neuron” includes a plurality of such neurons and reference to “the measurement” includes reference to one or more measurements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

The terms “individual”, “subject”, “host”, and “patient”, are used interchangeably herein and refer to any subject with a brain, including invertebrates and vertebrates such as, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

The term “user” as used herein refers to a person that interacts with a device and/or system disclosed herein for performing one or more steps of the presently disclosed methods. The user may be the patient receiving treatment. The user may be a health care practitioner, such as, the patient's physician.

The term “synucleinopathy” includes any disease associated with alpha-synuclein aggregation. The term includes neurodegenerative diseases associated with pathological accumulation of aggregates of alpha-synuclein in neurons or glia. Synucleinopathies include, but are not limited to, Parkinson's disease, dementia with Lewy bodies, multiple system atrophy, neuroaxonal dystrophies such as infantile neuroaxonal dystrophy and Hallervorden-Spatz syndrome, Shy-Drager syndrome, striatonigral degeneration, and olivopontocerebellar atrophy. The term also includes neurodegenerative diseases in which alpha-synuclein lesions contribute to pathological progression of the disease but are not the major protein constituent of lesions associated with the disease, such as Alzheimer's disease, amyotrophic lateral sclerosis, and frontotemporal dementia. Certain mutations cause alpha-synuclein to form amyloid-like fibrils that contribute to pathogenesis of disease. For example, the mutations, A53T, A30P, E46K, H50Q, and G51 D in alpha-synuclein are linked to Parkinson's disease.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with a synucleinopathy) as well as those in which prevention is desired (e.g., those with increased susceptibility to developing a synucleinopathy, those with a genetic predisposition to developing a synucleinopathy, those suspected of having a synucleinopathy, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Neural activity” as used herein, may refer to electrical activity of a neuron (e.g., changes in membrane potential of the neuron), as well as indirect measures of the electrical activity of one or more neurons. Thus, neural activity may refer to changes in field potential, changes in intracellular ion concentration (e.g., intracellular calcium concentration), and changes in magnetic resonance induced by electrical activity of neurons, as measured by, e.g., blood oxygenation level dependent (BOLD) signals in functional magnetic resonance imaging.

The term “survival” as used herein means the time from the start of treatment to the time of death.

The term “animal” is used herein to include all vertebrate animals, except humans. The term also includes animals at all stages of development, including embryonic, fetal, neonate, and adult stages. Animals may include any member of the subphylum Chordata, including, without limitation, non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### Systems and Computer Implemented Methods

The present disclosure provides systems and computer implemented methods which find use in practicing the subject methods. In some embodiments, the system may include: a processor programmed to predict locations where pathological protein aggregates will develop in the brain of a subject who has a neurological or a neurodegenerative disease, and a display component for displaying information regarding the predicted locations of the pathological protein aggregates in the brain of the subject. The system may also comprise one or more graphic boards for processing and outputting graphical information to the display component. For example, the display may be used to display an image of the brain of the subject showing the current locations of the pathological protein aggregates and/or the predicted past, present, or future locations of the pathological protein aggregates as determined by a computer implemented method.

In some embodiments, a computer implemented method is used for predicting locations wherein pathological protein aggregates will develop in the brain of a subject who has a neurological or a neurodegenerative disease. The processor can be programmed to perform steps of a computer implemented method comprising: a) receiving an image of the brain of the subject; b) identifying pathological protein aggregates in the image using a machine learning algorithm; c) mapping the positions of the pathological protein aggregates to neuroanatomical regions; d) modeling a discretized distribution of the pathological protein aggregates in each neuroanatomical region as a set of differential equations using a Smoluchowski network model; e) using regional gene density maps for each neuroanatomical region to calculate a distribution of gene effects on regional spreading and decay of the pathological protein aggregates; f) predicting changes in regional density of the pathological protein aggregates as a function of time by modeling spreading, aggregation, decay, and the spatial gene expression of the pathological protein aggregates throughout the neuroanatomical regions, wherein spreading is assumed to occur retrogradely between anatomically interconnected neuroanatomical regions, wherein spreading is modeled as diffusion through a weighted directed graph connecting the neuroanatomical regions of the brain of the subject; and g) predicting past locations, present locations, and future locations of the pathological protein aggregates based on said modeling.

In certain embodiments, the computer implemented method further comprises adjusting one or more programmed neurostimulation parameters based on said predicting the changes in regional density of the pathological protein aggregates as a function of time. For example, the duration, amplitude, frequency, pulse width, and location of the neurostimulation, or any combination thereof may be adjusted.

In certain embodiments, the computer implemented method further comprises instructing a neurostimulation device to apply neurostimulation to locations of the brain where pathological protein aggregates are predicted to be present in order to treat the neurological or neurodegenerative disease in the subject.

In certain embodiments, the computer implemented method further comprises instructing the neurostimulation device to apply electrical stimulation to locations of the brain where pathological protein aggregates are not yet present but are predicted to occur at a future time based on said predicting changes in the regional density of the pathological protein aggregates as a function of time.

In certain embodiments, the method further comprises: performing image registration to a coordinate space comprising a plurality of voxels, wherein each voxel is represented as a cubic volumetric element centered at a coordinate in the coordinate space; identifying positions in x, y, z coordinates of each pathological protein aggregate in the coordinate space; measuring volumes of each pathological protein aggregate from total number of voxels occupied by each pathological protein aggregate; calculating aggregate density for each voxel, wherein the aggregate density for each voxel is determined from total number of pathological protein aggregates having centers within the same voxel; calculating total aggregate size for each voxel, wherein the total aggregate size is the total size of all the pathological protein aggregates having centers within the same voxel; calculating mean aggregate size for each voxel as the total aggregate size divided by the aggregate density for each voxel; calculating total signal intensity for each voxel from the total intensity of all the pathological protein aggregates having centers within the same voxel; and calculating mean signal intensity for each voxel as the total signal intensity divided by the aggregate density for each voxel.

In certain embodiments, modeling of the discretized distribution of the pathological protein aggregates in each neuroanatomical region is performed using a Smoluchowski network model with the following set of differential equations:

$$\frac{d {c}_{1 , j}}{dt} = - α {\sum }_{k = 1}^{V} {L}_{jk} {c}_{1 , k} - {μ}_{1 , j} {c}_{1 , j} - 2 {c}_{1 , j}^{2} ( {κ}_{j} + ξ {\sum }_{k = 2}^{N} k {c}_{2 , k} ) - {c}_{1 , j} {\sum }_{k = 2}^{N} {c}_{k , j} , \\ \frac{d {c}_{2 , j}}{dt} = - {2}^{- η} α {\sum }_{k = 1}^{V} {L}_{jk} {c}_{2 , k} - {2}^{λ} {μ}_{2 , j} {c}_{2 , j} + {c}_{1 , j}^{2} ( {κ}_{j} + ξ {\sum }_{k = 2}^{N} {kc}_{2 , k} ) - {c}_{1 , j} {c}_{2 , j} , \\ \frac{d {c}_{i , j}}{dt} = - {i}^{- η} α {\sum }_{k = 1}^{V} {L}_{jk} {c}_{i , k} - {i}^{λ} {μ}_{ij} {c}_{ij} + {c}_{1 , j} ( {c}_{i - 1 , j} - ci , j ) , and \\ \frac{d {c}_{N , j}}{dt} = - {N}^{λ} {μ}_{N , j} {c}_{N , j} + {c}_{1 , j} {c}_{N - 1 , j} ,$$

wherein c_{i,j} represents the total count of pathological protein aggregates in a discretized size-bin indexed by l, in a brain region indexed by j, wherein an L matrix represents the Laplacian matrix of the weighted directed graph connecting the neuroanatomical regions of the brain, wherein η is chosen as a hyperparameter that slows the spread of large aggregates as the inverse power of the size, and wherein λ is chosen as a hyperparameter that accelerates the decay of pathological protein aggregates proportionally to the power of their size.

In certain embodiments, initial values for α and μ are fit by sweeping through a 2-dimensional-grid and selecting values that result in a lowest mean-squared error between predicted and actual counts of the pathological protein aggregates.

In certain embodiments, the initial value of μ=0, the initial value of k=0, ξ=0, and c is a one-dimensional size vector, wherein the set of differential equations simplifies to a standard network diffusion model.

In certain embodiments, the computer implemented method further comprises quantifying sensitivity of the model to specific neuroanatomical pathways in the brain, wherein a Jacobian matrix is calculated by taking a partial derivative of the model's output with respect to the weight of the anatomical connection strength between two neuroanatomical regions encoded into the model, wherein an element of the Jacobian matrix represents relative contribution of the anatomical connection between the two neuroanatomical regions to the spreading of pathological protein aggregates to a specific region of the brain.

In certain embodiments, the computer implemented method further comprises using the model to produce a ranking of candidate seed locations for a given pathological state c at t=T months post-injection (MPI) by a method comprising: using each of the neuroanatomical regions as separate seed locations at t=0 within the model and simulated forward in time to t=T MPI, wherein each of the simulation results for the different neuroanatomical regions are compared with an observed state c using a pairwise similarity metric, wherein the similarity metric is a correlation coefficient between total regional aggregate counts across observed and simulated states; and using the similarity metric values to sort the seed locations for the neuroanatomical regions as likely sites that lead to the observed pathological state c, wherein the ranking of candidate seed locations for the given pathological state c at t=T MPI is produced.

In certain embodiments, the computer implemented method further comprises predicting the time since seeding t=T MPI for a given pathological state c by a method comprising: comparing the whole-brain distribution of aggregate sizes for state c with simulated distributions at various t using a pairwise similarity metric without taking the seed location into account, wherein the distribution of simulated aggregate sizes across the whole brain is assumed to be invariant with respect to which neuroanatomical region is used as the seed location at t=0; and calculating the mean squared error between the stimulated and observed distributions, wherein when deciding among several candidate t values, the mean squared errors are inverted and normalized to sum to 1 to provide a prediction probability for each t being the correct estimate of T for the given pathological state c.

In certain embodiments, the gene effects on regional spreading and decay of the pathological protein aggregates are determined by a method comprising: assuming that α (spreading) and μ (decay) parameters are regionally dependent, wherein the spreading from a specific neuroanatomical region is proportional to the gene density in that region; normalizing all genes to the same range so that only the regional distribution of gene expression relative to that gene's total whole-brain expression is compared, wherein a is a vector, and the product of a with the Laplacian connectivity matrix L has the effect of modifying the regional connectivity encoded into the model; and normalizing each gene vector to have a mean of 1 and a standard deviation E that is empirically set to preserve a correlation between predicted and observed whole-brain aggregate count, wherein the normalization is chosen so that the product has the effect of maintaining the trace of the original Laplacian connectivity matrix L. In some embodiments, derivation of the normalization for maintaining the trace of the original Laplacian connectivity matrix L comprises: assuming the vector s is sampled from a multivariate normal distribution with mean-value 1 and standard deviation Σ, wherein s˜N(1,Σ); using a definition of the matrix trace and representing s as a diagonal square matrix S, wherein the trace of the product of S and the Laplacian connectivity matrix L results in the following:

$$Tr ( SL ) = Tr ( [ \begin{matrix}{s}_{1} & ⋯ & 0 \\ ⋮ & ⋱ & ⋮ \\ 0 & ⋯ & {s}_{V}\end{matrix} ] L ) = {\sum }_{i = 1}^{V} {s}_{i} {L}_{ii} = s \cdot diag ( L ) = s \cdot l ,$$

wherein l represents the diagonal of L, and wherein the trace is equivalent to the dot product of s and l, which has an expectation value equivalent to the sum of the entries of l, which recovers the definition of the trace of L according to the following equations:

$$s \cdot l ∼ N ( 1 \cdot l , l \sum l ) \\ E [ s \cdot l ] = Tr ( L ) ,$$

wherein after each gene is encoded into the model; comparing net effects on the regional correlation between the simulated and actual data to the baseline correlation with no genes; and providing an ordered list of genes ranked by the relevance of their spatial expression map in improving the regional predictions of the model.

In certain embodiments, the cubic volumetric element has a width of 100 μm in the coordinate space.

In certain embodiments, the one or more pathological protein aggregates map to a single voxel.

In certain embodiments, the computer implemented method further comprises performing multidimensional Gaussian filtering to account for variations in image registration between different samples.

In certain embodiments, the computer implemented method further comprises segmenting the image to produce a plurality of image segments.

In certain embodiments, the locations of the pathological protein aggregates are mapped to neuroanatomical regions of the Allen Human Brain Reference Atlas. In some embodiments, mapping comprises performing image registration to transform the locations of the pathological protein aggregates to the Allen Human Brain Reference Atlas coordinate space. In some embodiments, anatomically interconnected neuroanatomical regions are identified from the Allen Connectivity Atlas.

In certain embodiments, the neuroanatomical regions are selected from an Anterior amygdalar area, Anterior cingulate area, dorsal part, Anterior cingulate area, ventral part, Nucleus accumbens, Anterodorsal nucleus, Anterior hypothalamic nucleus, Agranular insular area, dorsal part, Agranular insular area, posterior part, Agranular insular area, ventral part, Nucleus ambiguous, Anteromedial nucleus, dorsal part, Anteromedial nucleus, ventral part, Ansiform lobule, Accessory olfactory bulb, Anterior olfactory nucleus, Anterior pretectal nucleus, Arcuate hypothalamic nucleus, Dorsal auditory area, Primary auditory area, Ventral auditory area, Anteroventral nucleus of thalamus, Basolateral amygdalar nucleus, Basomedial amygdalar nucleus, Bed nuclei of the stria terminalis, Field CA1, Field CA2, Field CA3, Central amygdalar nucleus, Central lobule, Central lateral nucleus of the thalamus, Claustrum, Central linear nucleus raphe, Central medial nucleus of the thalamus, Cortical amygdalar area, anterior part, Cortical amygdalar area, posterior part, Caudoputamen, Superior central nucleus raphe, Culmen, Cuneiform nucleus, Dorsal cochlear nucleus, Dentate gyrus, Dorsomedial nucleus of the hypothalamus, Dentate nucleus, Dorsal peduncular area, Dorsal nucleus raphe, Ectorhinal area, Entorhinal area, lateral part, Entorhinal area, medial part, dorsal zone, Endopiriform nucleus, dorsal part, Endopiriform nucleus, ventral part, Flocculus, Fastigial nucleus, Frontal pole, cerebral cortex, Fundus of striatum, Globus pallidus, external segment, Globus pallidus, internal segment, Gigantocellular reticular nucleus, Gustatory areas, Intercalated amygdalar nucleus, Inferior colliculus, central nucleus, Inferior colliculus, dorsal nucleus, Inferior colliculus, external nucleus, Infralimbic area, Intermediodorsal nucleus of the thalamus, Inferior olivary complex, Interposed nucleus, Interpeduncular nucleus, Intermediate reticular nucleus, Lateral amygdalar nucleus, Lateral vestibular nucleus, Lateral dorsal nucleus of thalamus, Dorsal part of the lateral geniculate complex, Ventral part of the lateral geniculate complex, Lateral habenula, Lateral hypothalamic area, Lateral posterior nucleus of the thalamus, Lateral preoptic area, Lateral reticular nucleus, Lateral septal nucleus, caudal (caudodorsal) part, Lateral septal nucleus, rostral (rostroventral) part, Lateral septal nucleus, ventral part, Magnocellular nucleus, Magnocellular reticular nucleus, Mediodorsal nucleus of thalamus, Medullary reticular nucleus, dorsal part, Medullary reticular nucleus, ventral part, Medial amygdalar nucleus, Median preoptic nucleus, Medial geniculate complex, dorsal part, Medial geniculate complex, medial part, Medial geniculate complex, ventral part, Medial habenula, Medial mammillary nucleus, Main olfactory bulb, Primary motor area, Secondary motor area, Medial preoptic nucleus, Medial preoptic area, Medial pretectal area, Midbrain reticular nucleus, Medial septal nucleus, Medial vestibular nucleus Diagonal band nucleus, Nucleus incertus, Nucleus of the lateral lemniscus, Nucleus of the lateral olfactory tract, Nodulus (X), Nucleus of the optic tract, Nucleus of the posterior commissure, Nucleus of the solitary tract, Orbital area, lateral part, Orbital area, medial part, Orbital area, ventrolateral part, Olfactory tubercle, Posterior amygdalar nucleus, Piriform-amygdalar area, Periaqueductal gray, Parasubiculum, Parvicellular reticular nucleus, Parabrachial nucleus, Pontine central gray, Perirhinal area, Parafascicular nucleus, Paraflocculus, Pontine gray, Paragigantocellular reticular nucleus, dorsal part, Paragigantocellular reticular nucleus, lateral part, Posterior hypothalamic nucleus, Piriform area, Prelimbic area, Dorsal premammillary nucleus, Posterior complex of the thalamus, Posterior limiting nucleus of the thalamus, Postsubiculum, Peripeduncular nucleus, Pedunculopontine nucleus, Presubiculum, Paramedian lobule, Pontine reticular nucleus, caudal part, Pontine reticular nucleus, Nucleus prepositus, Principal sensory nucleus of the trigeminal, Parataenial nucleus, Posterior parietal association areas, Paraventricular hypothalamic nucleus, Paraventricular nucleus of the thalamus, Periventricular hypothalamic nucleus, posterior part, Periventricular hypothalamic nucleus, preoptic part, Pyramus (VIII), Retrochiasmatic area, Nucleus of reuniens, Rhomboid nucleus, Nucleus raphe magnus, Red nucleus, Midbrain reticular nucleus, retrorubral area, Retrosplenial area, lateral agranular part, Retrosplenial area, dorsal part, Retrosplenial area, ventral part, Reticular nucleus of the thalamus, Subparaventricular zone, Superior colliculus, motor related, Superior colliculus, sensory related, Septofimbrial nucleus, Substantia innominate, Simple lobule, Submedial nucleus of the thalamus, Substantia nigra, compact part, Substantia nigra, reticular part, Superior olivary complex, Subparafascicular area, Subparafascicular nucleus, magnocellular part, Subparafascicular nucleus, parvicellular part, Spinal vestibular nucleus, Spinal nucleus of the trigeminal, caudal part, Spinal nucleus of the trigeminal, interpolar part, Spinal nucleus of the trigeminal, oral part, Primary somatosensory area, barrel field, Primary somatosensory area, lower limb, Primary somatosensory area, mouth, Primary somatosensory area, nose, Primary somatosensory area, trunk, Primary somatosensory area, upper limb, Supplemental somatosensory area, Subthalamic nucleus, Subiculum, Supramammillary nucleus, Supratrigeminal nucleus, Superior vestibular nucleus, Temporal association areas, Postpiriform transition area, Tegmental reticular nucleus, Triangular nucleus of septum, Taenia tecta, Tuberal nucleus, Motor nucleus of trigeminal, Ventral anterior-lateral complex of the thalamus, Ventral cochlear nucleus, Facial motor nucleus, Visceral area, Anterolateral visual area, Anteromedial visual area, Lateral visual area, Primary visual area, Posterolateral visual area, posteromedial visual area, Ventral medial nucleus of the thalamus, Ventromedial hypothalamic nucleus, Ventral posterolateral nucleus of the thalamus, Ventral posteromedial nucleus of the thalamus, Ventral posteromedial nucleus of the thalamus, parvicellular part, Ventral tegmental area, and Hypoglossal nucleus.

In certain embodiments, the computer implemented method further comprises predicting where pathological protein aggregates originated in the brain of the subject.

In certain embodiments, the subject is a human subject, wherein the modeling spreading, aggregation, and decay of the pathological protein aggregates in the brain of the human subject uses a simulation generated based on measuring spreading, aggregation, and decay of the pathological protein aggregates in a non-human animal model, wherein pathology measured in the non-human animal model experimentally is used to design simulation parameters to predict the past locations, the present locations, and the future locations of the pathological protein aggregates in the human subject. In certain embodiments, the simulation further uses brain anatomy or brain function measured from the non-human animal to predict the past locations, the present locations, and the future locations of the pathological protein aggregates in the human subject.

In some embodiments, the non-human animal used to develop the simulation, is a non-human mammal. Such mammals include, but are not limited to, non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese.

In certain embodiments, the simulation further uses brain anatomy or brain function measured from other human subjects to design simulation parameters to predict the past locations, the present locations, and the future locations of the pathological protein aggregates in a human patient. In some embodiments, the pathological protein aggregates in the brains of other human subjects are monitored to provide experimental data regarding spreading, aggregation, and decay of the pathological protein aggregates over time, which is used to design simulation parameters to predict the past locations, the present locations, and the future locations of the pathological protein aggregates in the human patient. The pathological protein aggregates can be monitored, for example, using any suitable medical imaging technique such as, but not limited to, imaging is performed using computed tomography (CT), single photon emission computed tomography (SPECT), magnetic resonance imaging, functional magnetic resonance imaging, optogenetic functional magnetic resonance imaging, and positron emission tomography (PET).

In certain embodiments, the computer implemented method further comprises: receiving a second image of the brain, wherein the second image is taken after said neurostimulation is applied to the brain; repeating steps (b)-(o) using the second image; and displaying changes in total aggregate size for each voxel, volume of each pathological protein aggregate for each voxel, and aggregate density for each voxel in the image taken after said neurostimulation is applied to the brain of the subject compared to the image taken before said neurostimulation is applied to the brain of the subject.

In certain embodiments, the computer implemented method further comprises: receiving a second image of the brain, wherein the second image is taken after said neurostimulation is applied to the brain; repeating steps (b)-(o) using the second image; modulating one or more programmed neurostimulation parameters based on any changes in the past locations, present locations, or future locations where the pathological protein aggregates are predicted to occur; and instructing a neurostimulation device to apply modulated neurostimulation to the brain of the subject in order to treat the neurological or neurodegenerative disease in the subject.

In certain embodiments, the computer implemented method further comprises storing a user profile for the subject comprising information regarding the programmed neurostimulation parameters used to apply neurostimulation to the brain of the subject to treat the neurological or neurodegenerative disease based on where the computer implemented method predicts pathological protein aggregates are present or will develop at a future time.

Analyzing identifying pathological protein aggregates in the image of the brain may comprise the use of an algorithm or classifier. In certain embodiments, a machine learning algorithm is used to identify pathological protein aggregates in the image of the brain. The machine learning algorithm may comprise a supervised learning algorithm. Examples of supervised learning algorithms may include Average One-Dependence Estimators (AODE), Artificial neural network (e.g., Backpropagation), Bayesian statistics (e.g., I Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines (SVM), Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network (recurrent or convoluted), Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

In some embodiments, the machine learning algorithm uses artificial neural networks. In some embodiments, the machine learning algorithm uses a deep learning algorithm, which may include the use of convolutional neural networks, deep neural networks, recurrent neural networks, deep residual neural networks, long short-term memory networks, deep belief networks, multilayer perceptrons, or deep reinforcement learning, and the like. For a description of deep learning algorithms, see, e.g., Pedrycz et al. Deep Learning: Algorithms and Applications (Studies in Computational Intelligence Book 865, Springer, 2019), Goodfellow et al. Deep Learning (Adaptive Computation and Machine Learning series, The MIT Press, 2016), and Various Deep Learning Algorithms in Computational Intelligence (edited by Oscar Humberto Montiel Ross, Mdpi AG, 2023); herein incorporated by reference in their entireties.

In certain embodiments, the computer implemented method further comprises segmenting the image to produce a plurality of image segments. Any suitable method known in the art can be used for image segmentation, to facilitate identification of pathological protein aggregates. Automatic or semiautomatic image analysis methods may be used for image segmentation. Various factors can complicate image analysis, including noise, autofluorescence, low resolution, blur, unstable brightness, overlapping targets, unclear boundaries, deformation, etc. In some cases, human intervention may be needed to accurately identify pathological protein aggregates in an image. In cases in which a classifier is insufficient to automatically identify single pathological protein aggregates accurately, a human may outline at least some of the pathological protein aggregates in an image to produce a set of pathological protein aggregates that can be used to train machine learning algorithms. Various software programs are currently available for image segmentation, including, but not limited to, the Ilastik Toolkit, which uses a random forest classifier for cell segmentation, DeepCell, which uses a deep-learning algorithm utilizing deep convolutional neural networks for image segmentation, Open Segmentation Framework (OpSeF), which semi-automates image segmentation using deep learning convolutional neural networks with the user manually providing some training data, CellSeg, which uses a mask region-convolutional neural network (R-CNN) for image segmentation, CODEX image processing pipeline software, which uses reference cellular markers, a reference nuclear stain, and a reference membrane stain to aid image segmentation, and CellProfiler, which uses conventional thresholding to classify a pixel as foreground if it is brighter than a certain “threshold” intensity value (cells appear as bright objects on a dark background in fluorescent microscopy images), illumination correction, declustering, and watershed segmentation for segmentation of images. For a description of image segmentation techniques and software, see, e.g., Kreshuk et al. (2019) Methods Mol. Biol. 2040:449-463, Kreshuk et al. (2014) PLoS One 9(2):e87351, David A. Van Valen et al. (2016) PLoS Comput. Biol. 12(11):e1005177, Dobson et al. (2021) Curr. Protoc. 1(5):e89, Stirling et al. (2021) BMC Bioinformatics 22(1):433, Soliman (2015) Biol Proced Online 17:11, Schapiro et al. (2017) Nat. Methods 14:873-876, Ljosa et al. (2009) PLoS Comput. Biol. 5(12):e1000603, and Lee et al. (2022) BMC Bioinformatics 23(1):46; herein incorporated by reference.

Additional relevant clinic metrics may also be stored in the system, including, without limitation, the subject's age, height, body-mass-index (BMI), gender, weight, and/or diagnosis, or other metrics applicable to the present techniques. In some cases, such metrics may be obtained from the subject's electronic medical records (EMR) or another applicable cloud-based storage technique, or, in other cases, may be measured and subsequently stored in the subject's electronic medical records or another applicable cloud-based storage technique.

The methods can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a processor, a storage component (i.e., memory), a display component, and other components typically present in general purpose computers. In some embodiments, the processor is provided by a computer or handheld device (e.g., a cell phone or tablet). The storage component stores information accessible by the processor, including instructions that may be executed by the processor and data that may be retrieved, manipulated or stored by the processor.

The storage component includes instructions. For example, the storage component includes instructions for determining predicting locations wherein pathological protein aggregates will develop in the brain of a subject who has a neurological or a neurodegenerative disease according to the methods described herein. The computer processor is coupled to the storage component and configured to execute the instructions stored in the storage component in order to receive an image of the brain of the subject and analyze the image according to one or more algorithms, as described herein, to predict past locations, present locations, and future locations of the pathological protein aggregates in the brain of the subject.

The processor and/or memory may be operably connected to a display device, for example, via a wired, such as a Universal Serial Bus (USB) connection, or wireless connection, such as a Bluetooth connection. Any convenient display device, such as a liquid crystal display (LCD), light-emitting diode (LED) display, plasma (PDP) display, quantum dot (QLED) display or cathode ray tube display device may be used. The display component displays information regarding the locations of the pathological protein aggregates in the brain of the subject. In some embodiments, the display displays an image of the brain of the subject showing the predicted present locations, past locations, or future locations of the pathological protein aggregates, as determined by the computer implemented method. In some embodiments, the display displays information regarding the coordinates of each pathological protein aggregate, the volume of each pathological protein aggregate; the aggregate density for each voxel, the total aggregate size for each voxel, the mean aggregate size for each voxel, the total signal intensity for each voxel, the mean signal intensity for each voxel, or the mapping of the positions of the pathological protein aggregates to neuroanatomical regions, or any combination thereof. In some embodiments, the display displays information regarding the distribution of gene effects on regional spreading and decay of the pathological protein aggregates. In some embodiments, the display displays information regarding the predicted changes in regional density of the pathological protein aggregates as a function of time determined by the modeling of the spreading, aggregation, decay, and the spatial gene expression of the pathological protein aggregates throughout the neuroanatomical regions. In some embodiments, the display displays information regarding the predicted past locations, present locations, and future locations of the pathological protein aggregates based on the modeling.

The storage component may be of any type capable of storing information accessible by the processor, such as a hard-drive, memory card, ROM, RAM, DVD, CD-ROM, USB Flash drive, write-capable, and read-only memories. The processor may be a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

In some embodiments, the method can be performed using a cloud computing system. In these embodiments, images of the subject's brain can be exported to a cloud computer, which runs the program, and returns an output to the user.

Components of systems for carrying out the presently disclosed methods are further described in the examples below.

### Neuromodulation Therapy for Treatment of Neurological and Neurodegenerative Diseases

In some embodiments, the subject methods are used to treat a synucleinopathy, which may include any disease associated with alpha-synuclein aggregation. Pathological aggregates of alpha-synuclein may accumulate, for example, in neurons or glia. Synucleinopathies include, but are not limited to, Parkinson's disease, dementia with Lewy bodies, multiple system atrophy, neuroaxonal dystrophies such as infantile neuroaxonal dystrophy and Hallervorden-Spatz syndrome, Shy-Drager syndrome, striatonigral degeneration, and olivopontocerebellar atrophy. In addition, the subject methods can be used to treat neurodegenerative diseases in which alpha-synuclein lesions contribute to pathological progression of the disease but are not the major protein constituent of lesions associated with the disease, such as Alzheimer's disease, amyotrophic lateral sclerosis, and frontotemporal dementia. Certain mutations cause alpha-synuclein to form amyloid-like fibrils that contribute to pathogenesis of disease. For example, the mutations, A53T, A30P, E46K, H50Q, and G51D in alpha-synuclein are linked to Parkinson's disease.

Neuromodulation can be achieved using electrical stimulation (e.g., from implanted, clinically approved electrodes), transcranial magnetic stimulation, transcranial electrical stimulation, or focused ultrasound, among other techniques. For electrical stimulation, deep brain stimulators can be used. Cortical layers or cell-types may be targeted specifically with genetically encodable modulation techniques, such as optogenetics. Deep brain stimulation (DBS), transcranial magnetic stimulation, transcranial electrical stimulation, or optogenetics may be used for neuromodulation of specific neuronal cell-types or neuronal circuits within the brain at locations where pathological protein aggregates are predicted to occur in the present, past, or future. For instance, parameters such as duration and site of neuromodulation can be tailored to patients based on their current pathological state and a neuromodulation parameter's expected impact on the pathology. Expected future states of pathology can also be taken into consideration in choosing neuromodulation parameters. One or more neurostimulation therapy parameters, including, but not limited to, the location, strength, and frequency of neurostimulation can be adjusted accordingly to treat neuropathology and reduce aggregation and spreading. Methods of neuromodulation are described in further detail below.

### Electrical Stimulation

In certain embodiments, electrical stimulation is applied to the brain of a subject using an electrode. The method includes positioning an electrode in a region of the brain of a subject to deliver electrical stimulation to the brain to disrupt aggregation and/or spreading of pathological protein aggregates to prevent or delay disease progression. The electrodes may be non-brain penetrating surface electrodes, extracranial electrodes, for example, subgaleal or skull mounted (in burrhole cap or in case of cranially mounted neurostimulator) or brain-penetrating depth electrodes. The electrical stimulation may be applied to the brain using the electrode in a manner effective for treating a neurological or neurodegenerative disease.

As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. An electrode can conduct electricity to specific targets in the brain. Electrodes used in the methods disclosed herein may be monopolar (cathode or anode) or bipolar (e.g., having an anode and a cathode).

Positioning an electrode may be carried out using standard surgical procedures for placement of intra-cranial electrodes. In certain cases, placing the electrode may involve positioning the electrode on the surface of specified region(s) of the brain where pathological protein aggregates are known to occur (e.g., based on medical imaging) or predicted to occur in the present, past, or future. The electrode may contact at least a portion of the surface of the brain at a specified region. In some embodiments, the electrode may contact substantially the entire surface area at the specified region. In some embodiments, the electrode may additionally contact area(s) adjacent to the specified region.

In some embodiments, an electrode array arranged on a planar support substrate may be used. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode, a subgaleal electrode, or an electroencephalography (EEG) electrode. In certain embodiments, a plurality of electrodes is positioned at one or more specified brain regions.

In certain cases, placing the electrode at a target area or site may involve positioning a brain penetrating electrode (also referred to as depth electrode) in specified region(s) of the brain. For example, an electrode may be placed in a region of the brain where pathological protein aggregates are known to occur (e.g., based on medical imaging) or are predicted to occur in the present, past, or future. In some embodiments, the electrode may additionally contact area(s) adjacent to a specified region of the brain. In some embodiments, one or more electrodes or electrode arrays are used to target one or more regions of the brain where pathological protein aggregates are predicted to occur in the present, past, or future.

The depth to which an electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

Positioning an electrode for delivering electrical stimulation to the brain may be carried out using standard surgical procedures for placement of electrodes for deep brain stimulation. For example, the electrode may be placed in a target region of the brain where pathological protein aggregates are known to occur (e.g., based on medical imaging) or predicted to occur in the present, past, or future. Medical imaging using, for example, magnetic resonance imaging (MRI) or computerized tomography (CT) may be used to provide guidance for placement of DBS electrodes and verify correct placement of the electrodes in the brain. In addition, a neurostimulator that generates electrical pulses is placed under the skin of the chest, typically below the collarbone or in the abdomen. In some embodiments the neurostimulator is cranially mounted. The surgical procedure may involve placing electrodes within the brain through small holes in the skull. An electrode lead is tunneled under the skin down the neck and under the skin of the chest to connect to a chest implanted neurostimulator.

Current is supplied by the neurostimulator to the electrodes. Parameters such as pulse width, shape, frequency, amplitude, pattern, and temporal distribution can be adjusted to modulate neural activity and neuronal circuits to reduce or prevent aggregation and spreading of pathological protein aggregates to treat a neurological or a neurodegenerative disease. In some embodiments, a closed loop system is used to adjust DBS settings automatically in response to changes in predicted locations of the pathological protein aggregates. In other embodiments, an open loop system is used in which DBS settings are adjusted by a user or medical practitioner based on the predicted locations of the pathological protein aggregates.

The electrical stimulation may be applied using a single electrode, electrode pairs, or an electrode array. In some embodiments, the number of electrodes used to deliver electrical stimulation to the brain ranges from 8 to 32, including any number of electrodes in this range such as 8, 10, 12, 14, 16, 18, 20, 22, 24, 26, 28, 30, or 32 electrodes. In some embodiments, the electrical stimulation is applied to more than one site. The site to which the electrical stimulation is applied may be alternated or otherwise spatially or temporally patterned. Electrical stimulation may be applied to the sites simultaneously or sequentially. The sites chosen for stimulation may differ for different subjects and will depend on where pathological protein aggregates are known to be present (e.g., by medical imaging) or predicted to be present.

In some embodiments, an electrode array arranged on a planar support substrate may be used for electrically stimulating the a region of the brain. The surface area of the electrode array may be determined by the desired area of contact between the electrode array and the brain. In some cases, cylindrical electrode arrays, paddle-style electrode arrays, or plate-style electrode arrays may be used in the methods disclosed herein for deep brain stimulation. Such electrode arrays for implanting in the brain, may be obtained from a commercial supplier. A commercially obtained electrode/electrode array may be modified to achieve a desired contact area.

The precise number of electrodes contained in an electrode array (e.g., for electrical stimulation) may vary. In certain aspects, an electrode array may include two or more electrodes, such as 3 or more, including 4 or more, e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, or about 96 or more electrodes. The electrodes may be arranged into a regular repeating pattern (e.g., a grid, such as a grid with about 1 cm spacing between electrodes), or no pattern. An electrode that conforms to the target site for optimal delivery of electrical stimulation may be used. One such example, is a single multi contact electrode with eight contacts separated by 2½ mm. Each contract would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three-pronged electrodes has four 1-2 mm contacts with a center to center separation of 2 of 2.5 mm and a span of 1.5 mm.

The size of each electrode may also vary depending upon such factors as the number of electrodes in the array, the location of the electrodes, the material, the age of the patient, and other factors. In certain aspects, an electrode array has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping the brain of the subject to optimize positioning of an electrode for applying electrical stimulation. Positioning of an electrode is optimized to maximize clinical responses to electrical stimulation to treat a neurological or neurodegenerative disease, which may include a synucleinopathy. In some embodiments, DBS is optimized to achieve a neurophysiologically defined change, for example, decreasing aggregation or alpha-synuclein, spreading of alpha-synuclein aggregates and/or improving brain function.

Assessment of the effectiveness of electrical stimulation at a particular site for treating a neurological or neurodegenerative disease may be performed using any standard method. In some embodiments, the effectiveness of electrical stimulation is assessed by imaging the brain of the subject to measure sizes and identify locations of the pathological protein aggregates after neurostimulation. In some embodiments, the effectiveness of electrical stimulation is assessed by measuring brain function of the subject after neurostimulation. For example, brain function may be measured by performing electroencephalography (EEG), stereoelectroencephalography (sEEG), electrocorticography (ECoG), magnetoencephalography (MEG), single photon emission computed tomography (SPECT), functional magnetic resonance imaging (fMRI), optogenetic functional magnetic resonance imaging, or positron emission tomography (PET).

In some cases, the severity of symptoms of a neurological or neurodegenerative disease may be further assessed using a visual analog scale or a verbal rating scale. In certain embodiments, the method further comprises assessing one or more motor and/or non-motor symptoms of the subject using a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale.

As set forth here, the subject methods involve applying electrical stimulation to a region of the brain where pathological protein aggregates are known to occur or predicted to occur in the present, past, or future. The parameters for applying the electrical stimulation to the brain may be determined empirically during treatment or may be pre-defined, such as, from a trial study with a subject. For example, varying stimulation settings may be applied including baseline (stimulation off), optimal therapeutic stimulation, modified and ineffective stimulation, and maximum tolerated stimulation to determine optimal therapeutic stimulation parameters for treatment of a neurological or neurodegenerative disease at sites where pathological protein aggregates are known to be present (e.g., by medical imaging) or predicted to occur according to the methods described herein.

The parameters of the electrical stimulation may include one or more of frequency, pulse width/duration, duty cycle, intensity/amplitude, pulse pattern, program duration, program frequency, and the like. In certain embodiments, the parameters are adjusted to target specific neuronal cell-types or neuronal circuits within the brain at locations where pathological protein aggregates are present or predicted to develop in the future.

Frequency refers to the pulses produced per second during stimulation and is stated in units of Hertz (Hz, e.g., 60 Hz=60 pulses per second). The frequencies of electrical stimulation used in the present methods may vary widely depending on numerous factors and may be determined empirically during treatment of the subject or may be pre-defined. In certain embodiments, the method may involve applying electrical stimulation to the brain at a frequency of 2 Hz-250 Hz, such as, 25 Hz-200 Hz, 50 Hz-250 Hz, 50 Hz-185 Hz, 50 Hz-150 Hz, 75 Hz-200 Hz, 100 Hz-200 Hz, 100 Hz-180 Hz, 100 Hz-160 Hz, or 130 Hz-150 Hz. In some embodiments, the electrical stimulation to the brain is applied at a frequency of about 120 Hz to about 160 Hz, including any pulse frequency within this range such as 120 Hz, 122 Hz, 124 Hz, 126 Hz, 128 Hz, 130 Hz, 132 Hz, 134 Hz, 136 Hz, 138 Hz, 140 Hz, 142 Hz, 144 Hz, 146 Hz, 148 Hz, 150 Hz, 152 Hz, 154 Hz, 156 Hz, 158 Hz, or 160 Hz. In some embodiments, non-integer pulse frequencies are used (e.g., 130.2 Hz, 130.4 Hz, etc.).

The electrical stimulation may be applied in pulses such as a uniphasic or a biphasic pulse. The time span of a single pulse is referred to as the pulse width or pulse duration. The pulse width used in the present methods may vary widely depending on numerous factors (e.g., severity of the disease, status of the patient, and the like) and may be determined empirically or may be pre-defined. In certain embodiments, the method may involve applying an electrical stimulation at a pulse width of about 10 μsec-500 μsec, for example, 20 μsec-450 μsec, 40 μsec-450 μsec, 60 μsec-450 μsec, 60 μsec-220 μsec, 60 μsec-120 μsec, or 60 μsec-90 μsec. In some embodiments, the electrical stimulation to the brain is applied at a pulse width of about 60 μsec to about 210 μsec, including any pulse width within this range such as 60 μsec, 65 μsec, 70 μsec, 75 μsec, 80 μsec, 85 μsec, 90 μsec, 95 μsec, 100 μsec, 105 μsec, 110 μsec, 115 μsec, 120 μsec, 125 μsec, 130 μsec, 135 μsec, 140 μsec, 145 μsec, 150 μsec, 155 μsec, 160 μsec, 165 μsec, 170 μsec, 175 μsec, 180 μsec, 185 μsec, 190 μsec, 195 μsec, 200 μsec, 205 μsec, 210 μsec, 215 μsec, or 220 μsec.

The electrical stimulation may be applied for a stimulation period of 0.1 μsec-1 month, with periods of rest (i.e., no electrical stimulation) possible in between. In certain cases, the period of electrical stimulation may be 0.1 μsec-1 week, 1 μsec-1 day, 10 μsec-12 hours, 1 min-6 hours, 10 min-1 hour, and so forth. In certain cases, the period of electrical stimulation may be 1 μsec-1 min, 1 μsec-30 μsec, 1 μsec-15 μsec, 1 μsec-10 μsec, 1 μsec-6 μsec, 1 μsec-3 μsec, 1 μsec-2 μsec, or 6 μsec-10 μsec. The period of rest in between each stimulation period may be 60 μsec or less, 30 μsec or less, 20 μsec or less, or 10 μsec. In some embodiments, electrical stimulation may be applied for a year or more, 2 years or more, 3 years or more, 5 years or more, or 10 years or more. In some embodiments, electrical stimulation may be continued indefinitely as part of a long-term DBS therapy regimen.

The electrical stimulation may be applied with an amplitude of current of 0.1 mA-30 mA, such as, 0.1 mA-25 mA, such as, 0.1 mA-20 mA, 0.1 mA-15 mA, 0.1 mA-10 mA, 0.1 mA-2 mA, 0.1 mA-1 mA, 1 mA-20 mA, 1 mA-10 mA, 2 mA-30 mA, 2 mA-15 mA, 2 mA-10 mA, or 1 mA-3 mA. In some embodiments, the amplitude of current is 0.1 mA-3.5 mA, or any amplitude of current in this range such as 0.1 mA, 0.2 mA, 0.3 mA, 0.4 mA, 0.5 mA, 0.6 mA, 0.7 mA, 0.8 mA, 0.9 mA, 1.0 mA, 1.1 mA, 1.2 mA, 1.3 mA, 1.4 mA, 1.5 mA, 1.6 mA, 1.7 mA, 1.8 mA. 1.9 mA, 2.0 mA, 2.1 mA, 2.2 mA, 2.3 mA, 2.4 mA, 2.5 mA, 2.6 mA, 2.7 mA, 2.8 mA, 2.9 mA, 3.0 mA, 3.1 mA, 3.2 mA, 3.3 mA, 3.4 mA, or 3.5 mA.

The electrical stimulation may be applied with an amplitude of voltage of 0.1 V-15 V, such as, 0.1 V-10 V, 0.1 V-5 V, 1 V-10 V, 1 V-5, V, or 1 V-3.5 V. In some embodiments, the amplitude of voltage is 1 V-3.5 V, or any amplitude of voltage in this range such as 1 V, 1.1 V, 1.2 V, 1.3 V, 1.4 V, 1.5 V, 1.6 V, 1.7 V, 1.8 V, 1.9 V, 2.0 V, 2.1 V, 2.2 V, 2.3 V, 2.4 V, 2.5 V, 2.6 V, 2.7 V, 2.8 V, 2.9 V, 3.0 V, 3.1 V, 3.2 V, 3.3 V, 3.4 V, or 3.5 V.

The electrical stimulation having the parameters as set forth above may be applied over a program duration of around 1 day or less, such as, 18 hours, 6 hours, 3 hours, 2 hours, 1 hour, 45 minutes, 30 minutes, 20 minutes, 10 minutes, or 5 minutes, or less, e.g., 1 minute-5 minutes, 2 minutes-10 minutes, 2 minutes-20 minutes, 2 minutes-30 minutes, 5 minutes-10 minutes, 5 minutes-30 minutes, or 5 minutes-15 minutes, 10 minutes-400 minutes, 25 minutes-300 minutes, 50 minutes-200 minutes, or 75 minutes-150 minutes, which period would include the application of pulses and the intervening rest period. The program may be repeated at a desired program frequency to reduce or prevent aggregation and spreading of pathological protein aggregates and relieve symptoms of a neurological or neurodegenerative disease in the subject. As such, a treatment regimen may include a program for electrical stimulation at a desired program frequency and program duration. In certain embodiments, the computer implemented method, described herein, is used to analyze images of the brain taken at different time points, wherein programmed neurostimulation parameters are adjusted based on any changes in the locations where the pathological protein aggregates are predicted to occur or develop. In some embodiments, the treatment regimen is controlled by a control unit in communication with a pulse generator connected to the one or more DBS electrodes in a closed-loop treatment regimen.

Upon completion of a treatment regimen, the patient may be assessed for effectiveness of the treatment and the treatment regimen may be repeated, if needed. In certain cases, the treatment regimen may be altered before repeating. For example, one or more of the frequency, pulse width, current amplitude, period of electrical stimulation, program duration, program frequency, and/or placement of DBS or detection electrodes may be altered before starting a second treatment regimen.

Application of the method may include a prior step of selecting a patient for treatment based on need as determined by clinical assessment, which may include assessment of severity of a neurological or neurodegenerative disease (e.g., a neurological or neurodegenerative disease lasting at least 3 months), physical condition, medication regime, cognitive assessment, anatomical assessment, behavioral assessment and/or neurophysiological assessment. In certain cases, a subject may be further assessed to determine if neurostimulation will completely or partially (e.g., at least 50%) relieve the neurological or neurodegenerative disease. Such a patient may undergo neurostimulation on a temporary trial basis to determine if neurostimulation reduces or prevents the aggregation and spread of pathological protein aggregates or decreases the severity of symptoms of the neurological or neurodegenerative disease experienced by the patient.

### Optogenetics

In some embodiments, optogenetics is used in a manner effective to decrease or prevent aggregation and spreading of pathological protein aggregates to treat a neurological or neurodegenerative disease. Optogenetics is used to allow optical control of activation (i.e., depolarization) or inhibition (i.e., hyperpolarization) of neurons that have been genetically modified to express light-responsive ion channels. In some embodiments, the light-responsive ion channel is a naturally occurring or synthetic opsin that uses a retinal-based cofactor (e.g., all-trans retinal for the microbial opsins) to respond to light. For example, light-responsive cation-conducting opsins (e.g., channelrhodopsin that conducts Ca²⁺) can be used to activate or depolarize neurons. Light-responsive anion-conducting opsins (e.g., channelrhodopsin or halorhodopsin that conduct chloride ions) or light-responsive proton conductance regulators (e.g., bacteriorhodopsin or archaerhodopsin) can be used to inhibit or hyperpolarize neurons. The levels of retinoids present in a mammalian brain are usually sufficient for expressed opsins to function without supplementation of cofactors. For a description of optogenetics and its use in controlling neural activity, see, e.g., Aravanis et al. (2007) J Neural Eng 4: S143-S156, Arenkiel et al. (2007) Neuron 54:205-218, Boyden et al. (2005) Nat Neurosci 8: 1263-1268, Chow et al. (2010) Nature 463:98-10, Gradinaru et al. (2007) J Neurosci 27:14231-14238, Gradinaru et al. (2008) Brain Cell Biol 36:129-139, Gradinaru et al. (2010) Cel/141:1-12, Li et al. (2005) Proc Natl Acad Sci 102:17816-17821, Lin et al. 2009. Characterization of engineered channelrhodopsin variants with improved properties and kinetics. Biophys J 96: 1803-1814, Yizhar et al. (2011) Microbial opsins: A family of single-component tools for optical control of neural activity. Cold Spring Harbor Protoc, Zhang et al. (2007) Nat Methods 4:139-141, Zhang et al. (2006) Nat Methods 3:785-792, Zhang et al. (2007) Nature 446: 633-639, Zhang et al. (2008) Nat Neurosci 11: 631-633; and U.S. Pat. Nos. 10,914,803; 10,589,123; 10,583,309; 10,568,516; 10,568,307; 10,538,560; 10,478,499; 10,220,092; 10,196,431; 10,087,223; 10,052,383; 9,969,783; 9,878,176; 9,855,442; 9,757,587; 9,458,208; and 8,834,546; herein incorporated by reference in their entireties.

In some embodiments, a target neuron is genetically modified to express a light-responsive ion channel that, when stimulated by an appropriate light stimulus, hyperpolarizes or depolarizes the stimulated target neuron. The term “genetic modification” refers to a permanent or transient genetic change induced in a cell following introduction into the cell of a heterologous nucleic acid (i.e., nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include the use of viral infection, transfection, conjugation, protoplast fusion, electroporation, particle gun technology, calcium phosphate precipitation, direct microinjection, and the like.

In some cases, a target cell that expresses a light-responsive polypeptide can be activated or inhibited upon exposure to light of varying wavelengths. In some cases, a target cell that expresses a light-responsive polypeptide is a neuronal cell that expresses a light-responsive polypeptide, and exposure to light of varying wavelengths results in depolarization or polarization of the neuron.

In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the excitable cell when activated by light of an activating wavelength. In some embodiments, the light-responsive polypeptide hyperpolarizes the excitable cell when activated by light of an activating wavelength.

In some cases, a light-responsive polypeptide mediates a hyperpolarizing current in the target cell it is expressed in when the cell is illuminated with light. Non-limiting examples of light-responsive polypeptides capable of mediating a hyperpolarizing current can be found, e.g., in U.S. Pat. Nos. 9,359,449 and 9,175,095. Non-limiting examples of hyperpolarizing light-responsive polypeptides include NpHr, eNpHr2.0, eNpHr3.0, eNpHr3.1 or GtR3. In some cases, a light-responsive polypeptide mediates a depolarizing current in the target cell it is expressed in when the cell is illuminated with light. Non-limiting examples of depolarizing light-responsive polypeptides include “C1V1”, ChR1, VChR1, ChR2. Additional information regarding other light-responsive cation channels, anion pumps, and proton pumps can be found in U.S. Patent Application Publication No: 2009/0093403; and U.S. Pat. No. 9,359,449.

In some embodiments, the light-responsive polypeptide can be activated by blue light (e.g., in range of 490 nm-450 nm). In one embodiment, the light-responsive polypeptide can be activated by light having a wavelength of about 473 nm. In some embodiments, the light-responsive polypeptide can be activated by yellow light (e.g., in range of 590 nm-560 nm). In another embodiment, the light-responsive polypeptide can be activated by light having a wavelength of about 560 nm. In another embodiment, the light-responsive polypeptide can be activated by red light (e.g., in range of 700 nm-635 nm). In another embodiment, the light-responsive polypeptide can be activated by light having a wavelength of about 630 nm. In other embodiments, the light-responsive polypeptide can be activated by violet light (e.g., in range of 450 nm-400 nm). In one embodiment, light-responsive polypeptide can be activated by light having a wavelength of about 405 nm. In other embodiments, the light-responsive polypeptide can be activated by green light (e.g., in range of 560 nm-520 nm). In other embodiments, the light-responsive polypeptide can be activated by cyan light (e.g., in range of 520 nm-490 nm). In other embodiments, the light-responsive polypeptide can be activated by orange light (e.g., in range of 635 nm-590 nm). A person of skill in the art would recognize that each light-responsive polypeptide will have its own range of activating wavelengths.

In some cases, the regions of the brain with neurons containing a light-responsive polypeptide are illuminated using one or more optical fibers. The optical fiber may be configured in any suitable manner to direct a light emitted from a suitable source of light, e.g., a laser or light-emitting diode (LED) light source, to the region of the brain. The optical fiber may be any suitable optical fiber. In some cases, the optical fiber is a multimode optical fiber. The optical fiber may include a core defining a core diameter, where light from the light source passes through the core. The optical fiber may have any suitable core diameter. In some cases, the core diameter of the optical fiber is 10 mm or more, e.g., 20 mm or more, 30 mm or more, 40 mm or more, 50 mm or more, 60 mm or more, including 80 mm or more, and is 1,000 mm or less, e.g., 500 mm or less, 200 mm or less, 100 mm or less, including 70 mm or less. In some embodiments, the core diameter of the optical fiber is in the range of 10 to 1,000 mm, e.g., 20 to 500 mm, 30 to 200 mm, including 40 to 100 mm.

The optical fiber end that is implanted into the target region of the brain may have any suitable configuration suitable for illuminating a region of the brain with a light stimulus delivered through the optical fiber. In some cases, the optical fiber includes an attachment device at or near the distal end of the optical fiber, where the distal end of the optical fiber corresponds to the end inserted into the subject. In some cases, the attachment device is configured to connect to the optical fiber and facilitate attachment of the optical fiber to the subject, such as to the skull of the subject. Any suitable attachment device may be used. In some cases, the attachment device includes a ferrule, e.g., a metal, ceramic or plastic ferrule. The ferrule may have any suitable dimensions for holding and attaching the optical fiber.

In certain embodiments, methods of the present disclosure may be performed using any suitable electronic components to control and/or coordinate the various optical components used to illuminate the regions of the brain. The optical components (e.g., light source, optical fiber, lens, objective, mirror, and the like) may be controlled by a controller, e.g., to coordinate the light source illuminating the regions of the brain with light pulses. The controller may include a driver for the light source that controls one or more parameters associated with the light pulses, such as, but not limited to the frequency, pulse width, duty cycle, wavelength, intensity, etc. of the light pulses. The controllers may be in communication with components of the light source (e.g., collimators, shutters, filter wheels, moveable mirrors, lenses, etc.).

In some embodiments, the light-responsive polypeptides are activated by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, or about 2 sec, inclusive, including any times in between these numbers. In some embodiments, the light-responsive polypeptides are activated by light pulses that can have a light power density of any of about 0.05 mW/mm², about 0.1 mW/mm², about 0.25 mW/mm², about 0.5 mW/mm², about 0.75 mW/mm², about 1 mW/mm², about 2 mW/mm², about 3 mW/mm², about 4 mW/mm², about 5 mW/mm², about 6 mW/mm², about 7 mW/mm², about 8 mW/mm², about 9 mW/mm², about 10 mW/mm², about 20 mW/mm², about 50 mW/mm², about 100 mW/mm², about 250 mW/mm², about 500 mW/mm², about 750 mW/mm², about 1000 mW/mm², about 1100 mW/mm², about 1200 mW/mm², about 1300 mW/mm², about 1400 mW/mm², about 1500 mW/mm², about 1600 mW/mm², about 1700 mW/mm², about 1800 mW/mm², about 1900 mW/mm², about 2000 mW/mm², about 2100 mW/mm², about 2200 mW/mm², about 2300 mW/mm², about 2400 mW/mm², about 2500 mW/mm², about 2600 mW/mm², about 2700 mW/mm², about 2800 mW/mm², about 2900 mW/mm², about 3000 mW/mm², about 3100 mW/mm², about 3100 mW/mm², about 3300 mW/mm², about 3400 mW/mm², or about 3500 mW/mm², inclusive, including any values between these numbers.

The light stimulus used to activate the light-responsive polypeptide may include light pulses characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc.

The light pulses may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 Hz or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some embodiments, the light pulses have a frequency in the range of 0.1 to 100,000 Hz, e.g., 1 to 10,000 Hz, 1 to 1,000 Hz, including 5 to 500 Hz, or 10 to 100 Hz.

In some cases, the two sets of light pulses are characterized by having different parameter values, such as different pulse widths, e.g. short or long. The light pulses may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The average power of the light pulse, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or

2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses may vary and may depend on the activation wavelength of the light-responsive polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

In some embodiments, the light-responsive polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-responsive protein transport to the plasma membranes of mammalian cells can be fused to the N-terminus, the C-terminus, or to both the N- and C-terminal ends of the light-responsive polypeptide. In some cases, the one or more amino acid sequence motifs which enhance light-responsive polypeptide transport to the plasma membranes of mammalian cells is fused internally within a light-responsive polypeptide. Optionally, the light-responsive polypeptide and the one or more amino acid sequence motifs may be separated by a linker. In some embodiments, the light-responsive polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In some embodiments, the signal peptide sequence in the protein can be deleted or substituted with a signal peptide sequence from a different protein.

Exemplary light-responsive polypeptides and amino acid sequence motifs that find use in the present system and method are disclosed in, e.g., U.S. Pat. Nos. 10,538,560; 10,568,307; 9,284,353; 9,359,449; and 9,365,628; herein incorporated by reference.

Light-responsive polypeptides of interest include, for example, a step function opsin (SFO)6 protein or a stabilized step function opsin (SSFO) protein that can have specific amino acid substitutions at key positions in the retinal binding pocket of the protein. See, for example, WO 2010/056970, the disclosure of which is hereby incorporated by reference in its entirety. The polypeptide may be a cation channel derived from Volvox carteri(VChR1), optionally comprising one or more amino acid substitutions, e.g., C123A; C123S; D151A, etc. A light-responsive cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1, optionally having an amino acid substitution at amino acid residue E122 or E162. In other embodiments, the light-responsive cation channel protein is a C1C2 chimeric protein derived from the ChR1 and the ChR2 proteins from Ch/amydomonas reinhardti, wherein the protein is responsive to light and is capable of mediating a depolarizing current in the cell when the cell is illuminated with light. In some embodiments, a depolarizing light-responsive polypeptide is a red shifted variant of a depolarizing light-responsive polypeptide derived from Chlamydomonas reinhardtii; referred to as a “ReaChR polypeptide” or “ReaChR protein” or “ReaChR.” In some embodiments, a depolarizing light-responsive polypeptide is a SdChR polypeptide derived from Scherffelia dubia, wherein the SdChR polypeptide is capable of transporting cations across a cell membrane when the cell is illuminated with light. In some embodiments, adepolarizing light-responsive polypeptide is CnChR1, derived from Chlamydomonas noctigama, wherein the CnChR1 polypeptide is capable of transporting cations across a cell membrane when the cell is illuminated with light. In some embodiments, the light-responsive cation channel protein is a CsChrimson chimeric protein derived from a CsChR protein of Chloromonas subdivisa and CnChR1 protein from Chlamydomonas noctigama, wherein the N-terminus of the protein comprises the amino acid sequence of residues 1-73 of CsChR followed by residues 79-350 of the amino acid sequence of CnChR1; is responsive to light; and is capable of mediating a depolarizing current in the cell when the cell is illuminated with light. In some embodiments, a depolarizing light-responsive polypeptide can be, e.g., ShChR1, derived from Stigeoclonium helveticum, wherein the ShChR1 polypeptide is capable of transporting cations across a cell membrane when the cell is illuminated with light.

In some embodiments, a depolarizing light-responsive polypeptide is derived from Chlamydomonas reinhardtii (CHR1, and particularly CHR2) wherein the polypeptide is capable of transporting cations across a cell membrane when the cell is illuminated with light; and is capable of mediating a depolarizing current in the cell when the cell is illuminated with light. In some embodiments CaMKIIa-driven, humanized channelrhodopsin CHR2 H134R mutant fused to EYFP is used for optogenetic activation. The light used to activate the light-responsive cation channel protein derived from Chlamydomonas reinhardtii can have a wavelength between about 460 and about 495 nm or can have a wavelength of about 480 nm. The light-responsive cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive cation channel protein to regulate the polarization state of the plasma membrane of the cell. Additionally, the light-responsive cation channel protein can comprise one or more conservative amino acid substitutions and/or one or more non-conservative amino acid substitutions. The light-responsive proton pump protein containing substitutions, deletions, and/or insertions introduced into the native amino acid sequence suitably retains the ability to transport cations across a cell membrane. The protein may comprise various amino acid substitutions, e.g., one or more of H134R; T159C; L132C; E123A; etc. The protein may further comprise a fluorescent protein, for example, but not limited to, a yellow fluorescent protein, a red fluorescent protein, a green fluorescent protein, or a cyan fluorescent protein.

Neurons can be selectively activated or inhibited optogenetically by engineering neurons to express one or more light-responsive polypeptides configured to hyperpolarize or depolarize the neurons. Suitable light-responsive polypeptides and methods used thereof are described further below.

A light-responsive polypeptide for use in the present disclosure may be any suitable light-responsive polypeptide for selectively activating neurons of a subtype by illuminating the neurons with an activating light stimulus. In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the cell when activated by light of an activating wavelength. In some embodiments, the light-responsive polypeptide hyperpolarizes the cell when activated by light of an activating wavelength. Suitable hyperpolarizing and depolarizing polypeptides are known in the art and include, e.g., a channelrhodopsin (e.g., ChR2), variants of ChR2 (e.g., C128S, D156A, C128S+D156A, E123A, E123T), iC12, C1C2, GtACR2, NpHR, eNpHR3.0, C1V1, VChR1, VChR2, SwiChR, Arch, ArchT, KR2, ReaChR, ChiEF, Chronos, ChRGR, CsChrimson, and the like. In some cases, the light-responsive polypeptide includes bReaCh-ES, as described in, e.g., Rajasethupathy et al., Nature. 2015 Oct. 29; 526(7575):653, which is incorporated by reference. Hyperpolarizing and depolarizing opsins have been described in various publications; see, e.g., Berndt and Deisseroth (2015) Science 349:590; Berndt et al. (2014) Science 344:420; and Guru et al. (Jul. 25, 2015) Intl. J. Neuropsychopharmacol. pp. 1-8 (PMID 26209858).

The light-responsive polypeptide may be introduced into the neurons using any suitable method. In some cases, the neurons of a subtype of interest are genetically modified to express a light-responsive polypeptide. In some cases, the neurons may be genetically modified using a viral vector, e.g., an adeno-associated viral vector, containing a nucleic acid having a nucleotide sequence that encodes the light-responsive polypeptide. The viral vector may include any suitable control elements (e.g., promoters, enhancers, recombination sites, etc.) to control expression of the light-responsive polypeptide according to neuronal subtype, timing, presence of an inducer, etc.

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non-protein coding sequence, e.g., a sequence encoding a light-reactive protein; and the like) if the promoter affects its transcription and/or expression.

Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956; see also, e.g., U.S. Pat. Nos. 6,649,811, 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor-.beta. promoter (see, e.g., Liu et al. (2620) Gene Therapy 11:52-60); a motor neuron-specific gene Hb9 promoter (see, e.g., U.S. Pat. No. 7,632,679; and Lee et al. (2620) Development 131:3295-3306); and an alpha subunit of Ca²⁺-calmodulin-dependent protein kinase II (CaMKII) promoter (see, e.g., Mayford et al. (1996) Proc. Natl. Acad. Sci. USA 93:13250). Other suitable promoters include elongation factor (EF) 1 and dopamine transporter (DAT) promoters.

In some cases, neuronal subtype-specific expression of the light-responsive polypeptide may be achieved by using recombination systems, e.g., Cre-Lox recombination, Flp-FRT recombination, etc. Cell type-specific expression of genes using recombination has been described in, e.g., Fenno et al., Nat Methods, 2014 July; 11(7):763; and Gompf et al., Front Behav Neurosci. 2015 Jul. 2; 9:152, which are incorporated by reference herein.

In some embodiments, the vector is a recombinant adeno-associated virus (AAV) vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Virol., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

It is understood that one or more vectors may be administered to neural cells. If more than one vector is used, it is understood that they may be administered at the same or at different times.

### Systems

The present disclosure also provides systems which find use, e.g., in practicing the subject methods. In certain embodiments, the system comprises a neurostimulation device and a processor programmed according to a computer implemented method, described herein, to instruct the neurostimulation device to deliver neurostimulation to the brain of a subject in a manner effective to treat a neurological or neurodegenerative disease in a subject, wherein neurostimulation is applied to the brain at known locations of the pathological protein aggregated (e.g. based on medical imaging) or at predicted present locations of the pathological protein aggregates, at predicted future locations of the pathological aggregates, or at predicted past locations of the pathological protein aggregates, or a combination thereof. The system may be an open-loop or closed-loop system configured for performing the methods provided herein. In some embodiments, the system may include a DBS electrode adapted for positioning at a region of the brain where pathological protein aggregates are known to be present (e.g., by medical imaging) or predicted to occur in the past, present, or future to deliver electrical stimulation to that region of the brain. In a closed-loop system, the system may also include a computing means and control unit programmed to instruct a DBS electrode to apply an electrical stimulation to a region of the brain where pathological protein aggregates are present or predicted to occur in a manner effective to treat a neurological or neurodegenerative disease in the subject. In certain embodiments, the neurostimulation intervention could take the form of non-invasive stimulation, including transcranial electrical stimulation or transcranial magnetic stimulation. In some embodiments, one or more programmed stimulation parameters are modulated according to an algorithm's control law based on where pathological protein aggregates are present or predicted to occur, and modulated electrical stimulation is delivered to the brain via the control unit, pulse generator and DBS electrode in a manner effective to treat a neurological or neurodegenerative disease. The closed loop system may include an on-body pulse generator that is connected to the implanted DBS electrodes and hence can apply electrical stimulation to the brain automatically upon receiving a communication from a control unit programmed according to the computer implemented methods described herein.

The processor of the closed-loop system may run programming as described herein for predicting locations wherein pathological protein aggregates will develop in the brain of a subject and/or assessing the effectiveness of treatment and modulate a parameter of the treatment as needed without user intervention. Thus, the closed-loop system may not necessarily include a user interface for a user to instruct the DBS electrode to apply an electrical stimulation to the brain to treat a neurological or neurodegenerative disease in the subject. However, in some embodiments, a user interface may be included in the closed-loop system which may be used to confirm the recommendation of the closed loop system, or to override it, or to change the recommendation.

Components of systems for carrying out the presently disclosed methods are further described in the examples below.

### Administration of a Pharmacological Agent

Embodiments of the methods and systems provided in this disclosure may also include administration of an effective amount of at least one pharmacological agent. By “effective amount” is meant a dosage sufficient to treat a neurological or neurodegenerative disease in a subject as desired. The effective amount will vary somewhat from subject to subject, and may depend upon factors such as the age and physical condition of the subject, type of neurological or neurodegenerative disease, severity of the neurological or neurodegenerative disease being treated, the duration of the treatment, the nature of any concurrent treatment, the form of the agent, the pharmaceutically acceptable carrier used if any, the route and method of delivery, and analogous factors within the knowledge and expertise of those skilled in the art. Appropriate dosages may be determined in accordance with routine pharmacological procedures known to those skilled in the art, as described in greater detail below.

If a pharmacological approach is employed in the treatment of a neurological or neurodegenerative disease, the specific nature and dosing schedule of the agent will vary depending on the particular nature of the disorder to be treated. Representative pharmacological agents that may find use in treatment of Parkinson's disease may include, but are not limited to, L-DOPA (l-3,4-dihydroxyphenylalanine, also known as levodopa), carbidopa (N-amino-a-methyl-3-hydroxy-L-tyrosine monohydrate), carbidopa-levodopa (Rytary, Sinemet, Duopa), a dopamine agonist, including, without limitation, pramipexole (Mirapex ER), rotigotine, apomorphine (Apokyn), and amantadine (Gocovri); a monoamine oxidase B (MAO-B) inhibitor, including, without limitation, selegiline (Zelapar), rasagiline (Azilect) and safinamide (Xadago); a catechol O-methyltransferase (COMT) inhibitor, including, without limitation, entacapone (Comtan), opicapone (Ongentys), and Tolcapone (Tasmar); an anticholinergic agent, including, without limitation, benztropine (Cogentin) and trihexyphenidyl; an adenosine receptor antagonist, including, without limitation an A2A receptor antagonist such as istradefylline (Nourianz), or an antipsychotic, including, without limitation, nuplazid (Pimavanserin), or any combination thereof.

In certain aspects, the administration of a pharmacological agent involves using a pharmacological delivery device such as, but not limited to, pumps (implantable or external devices), epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags. Administration of a pharmacological agent may be performed by a user or by a closed loop system.

### Kits

Also provided are kits comprising software for carrying out the computer implemented methods, described herein, for predicting locations wherein pathological protein aggregates will develop in the brain of a subject who has a neurological or a neurodegenerative disease and/or instructing a neurostimulation device to apply neurostimulation to locations of the brain where pathological protein aggregates are predicted to be present (e.g., in the past, present, and/or future) in order to treat the neurological or neurodegenerative disease in the subject. In some embodiments, the kit comprises a non-transitory computer-readable medium and instructions for treating a subject who has a neurological or a neurodegenerative disease based on locations where pathological protein aggregates are predicted to develop in the brain of a subject, using the computer implemented methods, as described herein. In some embodiments, the kit comprises a system comprising a processor programmed according to a computer implemented method described herein; and a display component for displaying information regarding the locations where pathological protein aggregates are predicted to develop in the brain of a subject.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The methods and systems of the present disclosure can be used to optimize neurostimulation therapy for altering pathology for treatment of a neurological or neurodegenerative disease. Computer implemented methods are provided to optimize neurostimulation therapy parameters including, without limitation, the location, strength, and frequency of neurostimulation. For example, the location of neurostimulation can be set based on where the algorithm predicts pathological protein aggregates to occur. Other parameters such as stimulation frequency and pulse width can subsequently be set to target specific neuronal cell-types or circuits within the brain.

In particular, the subject methods can be used to treat synucleinopathies, including any disease associated with alpha-synuclein aggregation. Synucleinopathies include neurodegenerative diseases associated with pathological accumulation of aggregates of alpha-synuclein in neurons or glia, such as, but not limited to, Parkinson's disease, dementia with Lewy bodies, multiple system atrophy, neuroaxonal dystrophies such as infantile neuroaxonal dystrophy and Hallervorden-Spatz syndrome, Shy-Drager syndrome, striatonigral degeneration, and olivopontocerebellar atrophy. The subject methods can also be used to treat neurodegenerative diseases in which alpha-synuclein lesions contribute to pathological progression of the disease but are not the major protein constituent of lesions associated with the disease, such as Alzheimer's disease, amyotrophic lateral sclerosis, and Pick disease.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-95 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A computer implemented method for predicting locations where pathological protein aggregates will develop in the brain of a subject who has a neurological or a neurodegenerative disease, the computer performing steps comprising:
a) receiving an image of the brain of the subject;
b) identifying pathological protein aggregates in the image using a machine learning algorithm;
c) mapping positions of the pathological protein aggregates to neuroanatomical regions;
d) modeling a discretized distribution of the pathological protein aggregates in each neuroanatomical region as a set of differential equations using a Smoluchowski network model;
e) using regional gene density maps for each neuroanatomical region to calculate a distribution of gene effects on regional spreading and decay of the pathological protein aggregates;
f) predicting changes in regional density of the pathological protein aggregates as a function of time by modeling spreading, aggregation, decay, and the spatial gene expression of the pathological protein aggregates throughout the neuroanatomical regions, wherein spreading is assumed to occur retrogradely between anatomically interconnected neuroanatomical regions, wherein spreading is modeled as diffusion through a weighted directed graph connecting the neuroanatomical regions of the brain of the subject; and
g) predicting past locations, present locations, and future locations of the pathological protein aggregates based on said modeling.

2. The computer implemented method of aspect 1, further comprising adjusting one or more programmed neurostimulation parameters based on said predicting the changes in regional density of the pathological protein aggregates as a function of time.

3. The computer implemented method of aspect 2, wherein the one or more programmed neurostimulation parameters are selected from duration, amplitude, frequency, pulse width, and location of the neurostimulation.

4. The computer implemented method of any one of aspects 1-3, further comprising instructing a neurostimulation device to apply neurostimulation to locations of the brain where pathological protein aggregates are predicted to be present in order to treat the neurological or neurodegenerative disease in the subject.

5. The computer implemented method of aspect 4, further comprising instructing the neurostimulation device to apply electrical stimulation to locations of the brain where pathological protein aggregates are not yet present but are predicted to occur at a future time based on said predicting changes in the regional density of the pathological protein aggregates as a function of time.

6. The computer implemented method of any one of aspects 1-5, further comprising: performing image registration to a coordinate space comprising a plurality of voxels, wherein each voxel is represented as a cubic volumetric element centered at a coordinate in the coordinate space;
identifying positions in x, y, z coordinates of each pathological protein aggregate in the coordinate space;
measuring volumes of each pathological protein aggregate from total number of voxels occupied by each pathological protein aggregate;
calculating aggregate density for each voxel, wherein the aggregate density for each voxel is determined from total number of pathological protein aggregates having centers within the same voxel;
calculating total aggregate size for each voxel, wherein the total aggregate size is the total size of all the pathological protein aggregates having centers within the same voxel;
calculating mean aggregate size for each voxel as the total aggregate size divided by the aggregate density for each voxel;
calculating total signal intensity for each voxel from the total intensity of all the pathological protein aggregates having centers within the same voxel; and
calculating mean signal intensity for each voxel as the total signal intensity divided by the aggregate density for each voxel.

7. The computer implemented method of any one of aspects 1-6, wherein said modeling the discretized distribution of the pathological protein aggregates in each neuroanatomical region as a set of differential equations using a Smoluchowski network model comprises using the following set of differential equations:

$$\frac{d {c}_{1 , j}}{dt} = - α {\sum }_{k = 1}^{V} {L}_{jk} {c}_{1 , k} - {μ}_{1 , j} {c}_{1 , j} - 2 {c}_{1 , j}^{2} ( {κ}_{j} + ξ {\sum }_{k = 2}^{N} k {c}_{2 , k} ) - {c}_{1 , j} {\sum }_{k = 2}^{N} {c}_{k , j} , \\ \frac{d {c}_{2 , j}}{dt} = - {2}^{- η} α {\sum }_{k = 1}^{V} {L}_{jk} {c}_{2 , k} - {2}^{λ} {μ}_{2 , j} {c}_{2 , j} + {c}_{1 , j}^{2} ( {κ}_{j} + ξ {\sum }_{k = 2}^{N} {kc}_{2 , k} ) - {c}_{1 , j} {c}_{2 , j} , \\ \frac{d {c}_{i , j}}{dt} = - {i}^{- η} α {\sum }_{k = 1}^{V} {L}_{jk} {c}_{i , k} - {i}^{λ} {μ}_{ij} {c}_{ij} + {c}_{1 , j} ( {c}_{i - 1 , j} - ci , j ) , and \\ \frac{d {c}_{N , j}}{dt} = - {N}^{λ} {μ}_{N , j} {c}_{N , j} + {c}_{1 , j} {c}_{N - 1 , j} ,$$

wherein c_{i,j} represents the total count of pathological protein aggregates in a discretized size-bin indexed by l, in a brain region indexed by j, wherein an L matrix represents the Laplacian matrix of the weighted directed graph connecting the neuroanatomical regions of the brain, wherein η is chosen as a hyperparameter that slows the spread of large aggregates as the inverse power of the size, and wherein λ is chosen as a hyperparameter that accelerates the decay of pathological protein aggregates proportionally to the power of their size.

8. The computer implemented method of aspect 7, wherein initial values for α and μ are fit by sweeping through a 2-dimensional-grid and selecting values that result in a lowest mean-squared error between predicted and actual counts of the pathological protein aggregates.

9. The computer implemented method of aspect 7, wherein the initial value of μ=0, the initial value of k=0, ξ=0, and c is a one-dimensional size vector, wherein the set of differential equations simplifies to a standard network diffusion model.

10. The computer implemented method of any one of aspects 7-9, further comprising quantifying sensitivity of the model to specific neuroanatomical pathways in the brain, wherein a Jacobian matrix is calculated by taking a partial derivative of the model's output with respect to the weight of the anatomical connection strength between two neuroanatomical regions encoded into the model, wherein an element of the Jacobian matrix represents relative contribution of the anatomical connection between the two neuroanatomical regions to the spreading of pathological protein aggregates to a specific region of the brain.

11. The computer implemented method of any one of aspects 7-10, further comprising using the model to produce a ranking of candidate seed locations for a given pathological state c at t=T months post-injection (MPI) by a method comprising:
using each of the neuroanatomical regions as separate seed locations at t=0 within the model and simulated forward in time to t=T MPI, wherein each of the simulation results for the different neuroanatomical regions are compared with an observed state c using a pairwise similarity metric, wherein the similarity metric is a correlation coefficient between total regional aggregate counts across observed and simulated states; and
using the similarity metric values to sort the seed locations for the neuroanatomical regions as likely sites that lead to the observed pathological state c, wherein the ranking of candidate seed locations for the given pathological state c at t=T MPI is produced.

12. The computer implemented method of aspect 11, further comprising predicting the time since seeding t=T MPI for a given pathological state c by a method comprising:
comparing the whole-brain distribution of aggregate sizes for state c with simulated distributions at various t using a pairwise similarity metric without taking the seed location into account, wherein the distribution of simulated aggregate sizes across the whole brain is assumed to be invariant with respect to which neuroanatomical region is used as the seed location at t=0; and
calculating the mean squared error between the stimulated and observed distributions, wherein when deciding among several candidate t values, the mean squared errors are inverted and normalized to sum to 1 to provide a prediction probability for each t being the correct estimate of T for the given pathological state c.

13. The computer implemented method of any one of aspects 7-12, wherein the gene effects on regional spreading and decay of the pathological protein aggregates are determined by a method comprising:
assuming that α (spreading) and μ (decay) parameters are regionally dependent, wherein the spreading from a specific neuroanatomical region is proportional to the gene density in that region;
normalizing all genes to the same range so that only the regional distribution of gene expression relative to that gene's total whole-brain expression is compared, wherein a is a vector, and the product of α with the Laplacian connectivity matrix L has the effect of modifying the regional connectivity encoded into the model; and
normalizing each gene vector to have a mean of 1 and a standard deviation Σ that is empirically set to preserve a correlation between predicted and observed whole-brain aggregate count, wherein the normalization is chosen so that the product has the effect of maintaining the trace of the original Laplacian connectivity matrix L.

14. The computer implemented method of aspect 13, wherein derivation of the normalization for maintaining the trace of the original Laplacian connectivity matrix L comprises:
assuming the vector s is sampled from a multivariate normal distribution with mean-value 1 and standard deviation Σ, wherein s˜N(1,Σ);
using a definition of the matrix trace and representing s as a diagonal square matrix S, wherein the trace of the product of S and the Laplacian connectivity matrix L results in the following:

$$Tr ( SL ) = Tr ( [ \begin{matrix}{s}_{1} & ⋯ & 0 \\ ⋮ & ⋱ & ⋮ \\ 0 & ⋯ & {s}_{V}\end{matrix} ] L ) = {\sum }_{i = 1}^{V} {s}_{i} {L}_{ii} = s \cdot diag ( L ) = s \cdot l ,$$

wherein l represents the diagonal of L, and wherein the trace is equivalent to the dot product of s and l, which has an expectation value equivalent to the sum of the entries of l, which recovers the definition of the trace of L according to the following equations:

$$s \cdot l ∼ N ( 1 \cdot l , l \sum l ) \\ E [ s \cdot l ] = Tr ( L ) ,$$

wherein after each gene is encoded into the model;
comparing net effects on the regional correlation between the simulated and actual data to the baseline correlation with no genes; and
providing an ordered list of genes ranked by the relevance of their spatial expression map in improving the regional predictions of the model.

15. The computer implemented method of any one of aspects 1-14, wherein the cubic volumetric element has a width of 100 μm in the coordinate space.

16. The computer implemented method of any one of aspects 1-15, wherein one or more pathological protein aggregates map to a single voxel.

17. The computer implemented method of any one of aspects 1-16, further comprising performing multidimensional Gaussian filtering to account for variations in image registration between different samples.

18. The computer implemented method of any one of aspects 1-17, further comprising segmenting the image to produce a plurality of image segments.

19. The computer implemented method of any one of aspects 1-18, wherein said mapping comprises mapping the locations of the pathological protein aggregates to neuroanatomical regions of the Allen Human Brain Reference Atlas.

20. The computer implemented method of aspect 19, wherein said mapping comprises performing image registration to transform the locations of the pathological protein aggregates to the Allen Human Brain Reference Atlas coordinate space.

21. The computer implemented method of aspect 19 or 20, wherein anatomically interconnected neuroanatomical regions are identified from the Allen Connectivity Atlas.

22. The computer implemented method of any one of aspects 1-21, wherein the neuroanatomical regions are selected from an Anterior amygdalar area, Anterior cingulate area, dorsal part, Anterior cingulate area, ventral part, Nucleus accumbens, Anterodorsal nucleus, Anterior hypothalamic nucleus, Agranular insular area, dorsal part, Agranular insular area, posterior part, Agranular insular area, ventral part, Nucleus ambiguous, Anteromedial nucleus, dorsal part, Anteromedial nucleus, ventral part, Ansiform lobule, Accessory olfactory bulb, Anterior olfactory nucleus, Anterior pretectal nucleus, Arcuate hypothalamic nucleus, Dorsal auditory area, Primary auditory area, Ventral auditory area, Anteroventral nucleus of thalamus, Basolateral amygdalar nucleus, Basomedial amygdalar nucleus, Bed nuclei of the stria terminalis, Field CA1, Field CA2, Field CA3, Central amygdalar nucleus, Central lobule, Central lateral nucleus of the thalamus, Claustrum, Central linear nucleus raphe, Central medial nucleus of the thalamus, Cortical amygdalar area, anterior part, Cortical amygdalar area, posterior part, Caudoputamen, Superior central nucleus raphe, Culmen, Cuneiform nucleus, Dorsal cochlear nucleus, Dentate gyrus, Dorsomedial nucleus of the hypothalamus, Dentate nucleus, Dorsal peduncular area, Dorsal nucleus raphe, Ectorhinal area, Entorhinal area, lateral part, Entorhinal area, medial part, dorsal zone, Endopiriform nucleus, dorsal part, Endopiriform nucleus, ventral part, Flocculus, Fastigial nucleus, Frontal pole, cerebral cortex, Fundus of striatum, Globus pallidus, external segment, Globus pallidus, internal segment, Gigantocellular reticular nucleus, Gustatory areas, Intercalated amygdalar nucleus, Inferior colliculus, central nucleus, Inferior colliculus, dorsal nucleus, Inferior colliculus, external nucleus, Infralimbic area, Intermediodorsal nucleus of the thalamus, Inferior olivary complex, Interposed nucleus, Interpeduncular nucleus, Intermediate reticular nucleus, Lateral amygdalar nucleus, Lateral vestibular nucleus, Lateral dorsal nucleus of thalamus, Dorsal part of the lateral geniculate complex, Ventral part of the lateral geniculate complex, Lateral habenula, Lateral hypothalamic area, Lateral posterior nucleus of the thalamus, Lateral preoptic area, Lateral reticular nucleus, Lateral septal nucleus, caudal (caudodorsal) part, Lateral septal nucleus, rostral (rostroventral) part, Lateral septal nucleus, ventral part, Magnocellular nucleus, Magnocellular reticular nucleus, Mediodorsal nucleus of thalamus, Medullary reticular nucleus, dorsal part, Medullary reticular nucleus, ventral part, Medial amygdalar nucleus, Median preoptic nucleus, Medial geniculate complex, dorsal part, Medial geniculate complex, medial part, Medial geniculate complex, ventral part, Medial habenula, Medial mammillary nucleus, Main olfactory bulb, Primary motor area, Secondary motor area, Medial preoptic nucleus, Medial preoptic area, Medial pretectal area, Midbrain reticular nucleus, Medial septal nucleus, Medial vestibular nucleus Diagonal band nucleus, Nucleus incertus, Nucleus of the lateral lemniscus, Nucleus of the lateral olfactory tract, Nodulus (X), Nucleus of the optic tract, Nucleus of the posterior commissure, Nucleus of the solitary tract, Orbital area, lateral part, Orbital area, medial part, Orbital area, ventrolateral part, Olfactory tubercle, Posterior amygdalar nucleus, Piriform-amygdalar area, Periaqueductal gray, Parasubiculum, Parvicellular reticular nucleus, Parabrachial nucleus, Pontine central gray, Perirhinal area, Parafascicular nucleus, Paraflocculus, Pontine gray, Paragigantocellular reticular nucleus, dorsal part, Paragigantocellular reticular nucleus, lateral part, Posterior hypothalamic nucleus, Piriform area, Prelimbic area, Dorsal premammillary nucleus, Posterior complex of the thalamus, Posterior limiting nucleus of the thalamus, Postsubiculum, Peripeduncular nucleus, Pedunculopontine nucleus, Presubiculum, Paramedian lobule, Pontine reticular nucleus, caudal part, Pontine reticular nucleus, Nucleus prepositus, Principal sensory nucleus of the trigeminal, Parataenial nucleus, Posterior parietal association areas, Paraventricular hypothalamic nucleus, Paraventricular nucleus of the thalamus, Periventricular hypothalamic nucleus, posterior part, Periventricular hypothalamic nucleus, preoptic part, Pyramus (VIII), Retrochiasmatic area, Nucleus of reuniens, Rhomboid nucleus, Nucleus raphe magnus, Red nucleus, Midbrain reticular nucleus, retrorubral area, Retrosplenial area, lateral agranular part, Retrosplenial area, dorsal part, Retrosplenial area, ventral part, Reticular nucleus of the thalamus, Subparaventricular zone, Superior colliculus, motor related, Superior colliculus, sensory related, Septofimbrial nucleus, Substantia innominate, Simple lobule, Submedial nucleus of the thalamus, Substantia nigra, compact part, Substantia nigra, reticular part, Superior olivary complex, Subparafascicular area, Subparafascicular nucleus, magnocellular part, Subparafascicular nucleus, parvicellular part, Spinal vestibular nucleus, Spinal nucleus of the trigeminal, caudal part, Spinal nucleus of the trigeminal, interpolar part, Spinal nucleus of the trigeminal, oral part, Primary somatosensory area, barrel field, Primary somatosensory area, lower limb, Primary somatosensory area, mouth, Primary somatosensory area, nose, Primary somatosensory area, trunk, Primary somatosensory area, upper limb, Supplemental somatosensory area, Subthalamic nucleus, Subiculum, Supramammillary nucleus, Supratrigeminal nucleus, Superior vestibular nucleus, Temporal association areas, Postpiriform transition area, Tegmental reticular nucleus, Triangular nucleus of septum, Taenia tecta, Tuberal nucleus, Motor nucleus of trigeminal, Ventral anterior-lateral complex of the thalamus, Ventral cochlear nucleus, Facial motor nucleus, Visceral area, Anterolateral visual area, Anteromedial visual area, Lateral visual area, Primary visual area, Posterolateral visual area, posteromedial visual area, Ventral medial nucleus of the thalamus, Ventromedial hypothalamic nucleus, Ventral posterolateral nucleus of the thalamus, Ventral posteromedial nucleus of the thalamus, Ventral posteromedial nucleus of the thalamus, parvicellular part, Ventral tegmental area, and Hypoglossal nucleus.

23. The computer implemented method of any one of aspects 1-22, further comprising predicting where pathological protein aggregates originated in the brain of the subject.

24. The computer implemented method of any one of aspects 1-23, wherein the machine learning algorithm uses an artificial neural network.

25. The computer implemented method of any one of aspects 1-24, wherein the machine learning algorithm uses a deep learning algorithm.

26. The computer implemented method of aspect 25, wherein the deep learning algorithm uses a convolutional neural network, a deep neural network, a recurrent neural network, a deep residual neural network, a long short-term memory network, a deep belief network, a multilayer perceptron, or deep reinforcement learning.

27. The computer implemented method of any one of aspects 1-26, wherein the machine learning algorithm is supervised, semi-supervised, or unsupervised.

28. The computer implemented method of any one of aspects 1-27, wherein the subject is a human subject.

29. The computer implemented method of aspect 28, wherein modeling spreading, aggregation, and decay of the pathological protein aggregates in the brain of the human subject uses a simulation generated based on measuring spreading, aggregation, and decay of the pathological protein aggregates in a non-human animal model, wherein pathology measured in the non-human animal model experimentally is used to design simulation parameters to predict the past locations, the present locations, and the future locations of the pathological protein aggregates in the human subject.

30. The computer implemented method of aspect 29, wherein the simulation further uses brain anatomy or brain function measured from the non-human animal to predict the past locations, the present locations, and the future locations of the pathological protein aggregates in the human subject.

31. The computer implemented method of aspect 29 or 30, wherein the non-human animal is a mammal.

32. The computer implemented method of aspect 31, wherein the mammal is a rodent or a primate.

33. The computer implemented method of aspect 32, wherein the rodent is a mouse.

34. The computer implemented method of any one of aspects 29-33, wherein the simulation further uses brain anatomy or brain function measured from other human subjects to design simulation parameters to predict the past locations, the present locations, and the future locations of the pathological protein aggregates in the human subject.

35. The computer implemented method of any one of aspects 1-34, further comprising:
receiving a second image of the brain, wherein the second image is taken after said neurostimulation is applied to the brain;
repeating steps (b)-(o) using the second image; and
displaying changes in the total aggregate size for each voxel, the volume of each pathological protein aggregate for each voxel, and the aggregate density for each voxel in the image taken after said neurostimulation is applied to the brain of the subject compared to the image taken before said neurostimulation is applied to the brain of the subject.

36. The computer implemented method of any one of aspects 1-34, further comprising:
receiving a second image of the brain, wherein the second image is taken after said neurostimulation is applied to the brain;
repeating steps (b)-(o) using the second image;
modulating one or more programmed neurostimulation parameters based on any changes in the past locations, present locations, or future locations where the pathological protein aggregates are predicted to occur; and
instructing a neurostimulation device to apply modulated neurostimulation to the brain of the subject in order to treat the neurological or neurodegenerative disease in the subject.

37. The computer implemented method of any one of aspects 1-36, further comprising storing a user profile for the subject comprising information regarding the programmed neurostimulation parameters used to apply neurostimulation to the brain of the subject to treat the neurological or neurodegenerative disease based on where the computer implemented method predicts pathological protein aggregates are present or will develop at a future time.

38. A non-transitory computer-readable medium comprising program instructions that, when executed by a processor in a computer, causes the processor to perform the method of any one of aspects 1-37.

39. A kit comprising the non-transitory computer-readable medium of aspect 38 and instructions for treating a neurological or a neurodegenerative disease in a subject with neurostimulation.

40. A method for treating a neurological or neurodegenerative disease in a subject, the method comprising:
imaging pathological protein aggregates in the brain of the subject;
using the computer implemented method of any one of aspects 1-37 to predict where pathological protein aggregates will develop based on locations of the pathological protein aggregates that are detected in the brain of the subject by said imaging; and
applying neurostimulation at locations in the brain where the pathological protein aggregates are detected in the brain of the subject by said imaging and at locations where the computer implemented method predicts pathological protein aggregates will develop.

41. The method of aspect 40, wherein said imaging is performed using computed tomography (CT), single photon emission computed tomography (SPECT), magnetic resonance imaging, functional magnetic resonance imaging, optogenetic functional magnetic resonance imaging, or positron emission tomography (PET).

42. The method of aspect 40 or 41, further comprising adjusting stimulation frequency and pulse width of the neurostimulation to target specific neuronal cell-types or circuits within the brain at the locations in the brain where the computer implemented method predicts the pathological protein aggregates are present or will develop at the future time.

43. The method of any one of aspects 40-42, wherein the neurological or neurodegenerative disease is a synucleinopathy.

44. The method of aspect 43, wherein the synucleinopathy is Parkinson's disease, dementia with Lewy bodies, multiple system atrophy, neuroaxonal dystrophy Shy-Drager syndrome, striatonigral degeneration, or olivopontocerebellar atrophy.

45. The method of any one of aspects 40-42, wherein the neurological or neurodegenerative disease is Alzheimer's disease, amyotrophic lateral sclerosis, or frontotemporal dementia.

46. The method of any one of aspects 40-45, wherein the pathological protein aggregates comprise alpha-synuclein aggregates.

47. The method of any one of aspects 40-46, wherein said applying neurostimulation comprises applying neurostimulation using an electrode.

48. The method of aspect 47, wherein the electrode is a depth electrode or a surface electrode.

49. The method of aspect 47, wherein the electrode is a non-brain penetrating surface electrode array or a brain-penetrating electrode array.

50. The method of any one of aspects 40-49, wherein said applying neurostimulation comprises applying deep brain stimulation, transcranial magnetic stimulation, or transcranial electrical stimulation.

51. The method of any one of aspects 40-46, wherein said applying neurostimulation comprises applying neurostimulation optogenetically.

52. The method of aspect 51, wherein neurostimulation is applied optogenetically by a method comprising:
introducing a recombinant polynucleotide encoding a light-responsive ion channel into a neuron at the location in the brain where the computer implemented method predicts pathological protein aggregates are present or will develop at a future time, wherein the light-responsive ion channel is expressed in the neuron; and
illuminating the light-responsive ion channel with light at a wavelength that activates the light-responsive ion channel, wherein conduction of ions by the light-responsive ion channel in response to absorption of light results in hyperpolarization or depolarization of the neuron.

53. The method of aspect 52, wherein the light-responsive ion channel is a light-responsive anion-conducting opsin or a light-responsive proton conductance regulator.

54. The method of aspect 53, wherein the light-responsive anion-conducting opsin conducts chloride ions (Cl⁻).

55. The method of aspect 53 or 54, wherein the anion-conduction opsin is an anion-conducting channelrhodopsin or halorhodopsin.

56. The method of aspect 55, wherein the halorhodopsin is a Natronomonas pharaonis halorhodopsin (NpHR), enhanced NpHR (eNpHR) 1.0, eNpHR 2.0, or eNpHR 3.0.

57. The method of aspect 55, wherein the anion-conducting channelrhodopsin is iC1C2, SwiChR, SwiChR++, or iC++.

58. The method of aspect 53, wherein the light-responsive proton conductance regulator is a bacteriorhodopsin or an archaerhodopsin.

59. The method of aspect 58, wherein the light-responsive proton conductance regulator is Arch from Halorubrum sodomense, ArchT from Halorubrum sp., TP009 from Leptosphaeria maculans, or Mac from Leptosphaeria maculans.

60. The method of aspect 52, wherein the light-responsive ion channel is a light-responsive cation-conducting opsin.

61. The method of aspect 60, wherein the light-responsive cation-conducting opsin conducts calcium cations (Ca²⁺).

62. The method of aspect 60 or 61, wherein the light-responsive cation-conducting opsin is a light-responsive cation-conducting channelrhodopsin.

63. The method of aspect 62, wherein the light-responsive cation-conducting channelrhodopsin is a Chlamydomonas reinhardtii channelrhodopsin or a Volvox carteri channelrhodopsin.

64. The method of aspect 63, wherein the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

65. The method of any one of aspects 52-64, wherein the polynucleotide encoding the light-responsive ion channel is provided by a viral vector.

66. The method of aspect 65, wherein the viral vector is a lentiviral vector or an adeno-associated viral (AAV) vector.

67. The method of aspect 65 or 66, wherein the viral vector is stereotactically injected into the brain at the location where the computer implemented method predicts pathological protein aggregates are present or will develop at a future time.

68. The method of any one of aspects 65-67, wherein the vector further comprises a neuron-specific promoter operably linked to the polynucleotide encoding the light-responsive ion channel.

69. The method of any one of aspects 52-68, wherein expression of the light-responsive ion channel is inducible.

70. The method of any one of aspects 52-69, wherein said illuminating the light-responsive ion channel comprises delivering light from a light source to the light-responsive ion channel using a fiber-optic-based optical neural interface.

71. The method of aspect 70, wherein the light source is a solid-state diode laser.

72. The method of any one of aspects 52-71, wherein said applying neurostimulation comprises applying neurostimulation to a motor cortex region or a subcortical region of the brain.

73. The method of any one of aspects 40-72, wherein multiple cycles of the neurostimulation are performed.

74. The method of any one of aspects 40-73, further comprising assessing effectiveness of the treatment of the neurological or neurodegenerative disease in the subject.

75. The method of aspect 74, wherein said assessing comprises imaging the brain of the subject to measure sizes and identify locations of the pathological protein aggregates after said neurostimulation.

76. The method of aspect 74 or 75, wherein said assessing comprises measuring brain function of the subject after said neurostimulation.

77. The method of aspect 76, wherein said measuring brain function comprises performing electroencephalography (EEG), stereoelectroencephalography (sEEG), electrocorticography (ECoG), magnetoencephalography (MEG), single photon emission computed tomography (SPECT), functional magnetic resonance imaging (fMRI), optogenetic functional magnetic resonance imaging, or positron emission tomography (PET).

78. The method of aspect 76 or 77, further comprising modulating one or more programmed neurostimulation parameters to improve the brain function.

79. The method of any one of aspects 74-78, further comprising assessing severity of symptoms of the neurological or neurodegenerative disease using a visual analog scale, a verbal rating scale, a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale

80. A system for treating a neurological or neurodegenerative disease in a subject, the system comprising:
a neurostimulation device; and
a processor programmed according to the computer implemented method of any one of aspects 1-37 to instruct the neurostimulation device to deliver neurostimulation to the brain of the subject in a manner effective to treat the neurological or neurodegenerative disease in the subject, wherein neurostimulation is applied to the brain at predicted present locations of the pathological protein aggregates, at predicted future locations of the pathological protein aggregates, or at predicted past locations of the pathological protein aggregates, or a combination thereof.

81. The system of aspect 80, wherein the neurostimulation device comprises an electrode.

82. The system of aspect 81, wherein the electrode is a depth electrode or a surface electrode.

83. The system of aspect 82, wherein the electrode is a non-brain penetrating surface electrode array or a brain-penetrating electrode array.

84. The system of any one of aspects 80-83, wherein the neurostimulation device performs deep brain stimulation, transcranial magnetic stimulation, or transcranial electrical stimulation.

85. The system of any one of aspects 80-84, further comprising a display.

86. The system of aspect 85, wherein the display displays an image of the brain of the subject showing the predicted present locations, past locations, or future locations of the pathological protein aggregates determined by the computer implemented method.

87. The system of aspect 85 or 86, wherein the display displays information regarding the coordinates of each pathological protein aggregate, the volume of each pathological protein aggregate; the aggregate density for each voxel, the total aggregate size for each voxel, the mean aggregate size for each voxel, the total signal intensity for each voxel, the mean signal intensity for each voxel, or the mapping of the positions of the pathological protein aggregates to neuroanatomical regions, or any combination thereof.

88. The system of any one of aspects 85-87, wherein the display displays information regarding the distribution of gene effects on regional spreading and decay of the pathological protein aggregates.

89. The system of any one of aspects 85-88, wherein the display displays information regarding the predicted changes in regional density of the pathological protein aggregates as a function of time determined by the modeling of the spreading, aggregation, decay, and the spatial gene expression of the pathological protein aggregates throughout the neuroanatomical regions.

90. The system of any one of aspects 85-89, wherein the display displays information regarding the predicted past locations, present locations, and future locations of the pathological protein aggregates based on the modeling.

91. The system of any one of aspects 85-90, wherein the system further comprises a user interface comprising an input electronically coupled to the processor for instructing the neurostimulation device to apply neurostimulation to the brain of the subject to treat the neurological or neurodegenerative disease in the subject.

92. The system of aspect 91, wherein the user interface is password protected and is operable by a health care practitioner.

93. The system of any one of aspects 85-92, wherein the neurological or neurodegenerative disease is a synucleinopathy.

94. The system of aspect 93, wherein the synucleinopathy is Parkinson's disease, dementia with Lewy bodies, multiple system atrophy, neuroaxonal dystrophy Shy-Drager syndrome, striatonigral degeneration, or olivopontocerebellar atrophy.

95. The system of any one of aspects 85-92, wherein the neurological or neurodegenerative disease is Alzheimer's disease, amyotrophic lateral sclerosis, or frontotemporal dementia.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Mesoscale Connections and Gene Expression Empower Whole-Brain Modeling of α-Synuclein Spread, Aggregation, and Decay Dynamics

Parkinson's disease (PD) is the second most common neurodegenerative disorder. It is characterized by postural instability, tremor, rigidity, and bradykinesia (Goetz, 2011; Kalia and Lang, 2015). These clinical manifestations are caused primarily by loss of dopaminergic neurons from the substantia nigra. The hallmark pathology of PD is the presence of Lewy bodies (LB), cytoplasmic neuronal inclusions composed of misfolded aggregates of the protein α-synuclein (α-syn) (Dickson, 2012; Goedert et al., 2013; Kalia and Lang, 2015; Oliveira et al., 2021; Spillantini et al., 1997). A longstanding hypothesis about the etiopathogenesis of this debilitating disease has been the Braak hypothesis—which posits that pathological α-syn seeds form early in the disease and subsequently spread through the nervous system, correlating with the progression of motor and cognitive symptoms (Beach et al., 2009; Braak et al., 2002; Braak et al., 2003). An exciting area in neurodegenerative disease research is the emerging phenomenon of prion-like spreading of neurodegenerative disease proteins, including α-syn in PD (Aguzzi and Rajendran, 2009; Angot et al., 2010; Cushman et al., 2010; Guo and Lee, 2014; Jucker and Walker, 2013). Prions are well established as the protein-based infectious agent underlying the spongiform encephalopathies (for example, bovine spongiform encephalopathy in cattle and Creutzfeldt-Jakob disease in humans). In these rare, albeit devastating, diseases, the prion protein, PrP, converts from the normal soluble form to the aggregated self-templating infectious form. This process initiates an inexorable spread of pathology and contingent neurodegeneration throughout the brain (Aguzzi and Calella, 2009; Prusiner, 1998). But could this phenomenon extend to the more common neurodegenerative diseases like PD?

Early hints of this type of possibility came from postmortem analyses of individuals who had received fetal nigral transplants as a PD treatment and then had subsequently died several years later and come to autopsy (Kordower et al., 2008; Li et al., 2008). In some of the fetal grafts (which were only 11-16 years old at the time of autopsy), Lewy bodies, comprised of α-syn fibrils, were present. The findings were startling because they suggested the possibility that somehow α-syn aggregates from the host diseased tissue propagated to the new graft tissue. These findings were consistent with the longstanding Braak hypothesis—that PD pathology seems to spread through the brain in anatomically defined ways (Braak et al., 2003).

Although several studies have questioned this “prion-like” hypothesis owing to its inability to explain the sparsity of staged distributions in post-mortem human PD brains (Surmeier et al., 2017), studies in primary neurons and animal models demonstrate that α-syn pathology can and does spread in a cell-to-cell manner, causing impairments in excitability and ultimately leading to neuronal degeneration (Desplats et al., 2009; Hansen et al., 2011; Volpicelli-Daley et al., 2011). Injecting α-syn fibrils into α-syn overexpressing transgenic mice causes pathology and degeneration (Luk et al., 2012b). Importantly, α-syn fibrils injected into wild-type mice causes spread of pathology along anatomically interconnected brain regions, decrease in tyrosine hydroxylase-positive dopaminergic neurons, and results in motor impairments (Luk et al., 2012a). Countering the argument that these results could be a secondary consequence of a signaling pathway induced by aggregated α-syn, injection of α-syn fibrils into α-syn knockout mice causes no spread of pathology, no degeneration, and no motor impairment. Furthermore, injection of fibrils in α-syn heterozygous mice causes a reduction in pathology and a reduction in motor impairments (Luk et al., 2012a). This proves that spreading is α-syn-dependent. These observations have been extended to rats, non-human primates, and in human neurons (Bieri et al., 2019; Gribaudo et al., 2019; Paumier et al., 2015; Prusiner et al., 2015; Recasens et al., 2014; Shimozawa et al., 2017). These results point to α-syn trans-neuronal spreading playing a significant role in neurodegeneration in PD.

A picture emerges in which a small amount of α-syn fibrillar seeds (either formed spontaneously in the human brain or by direct injection into the mouse brain) can template the conversion of endogenous α-syn to an aggregated state and set in motion a flywheel that drives the nervous system inexorably toward disease. The big challenge now is to define how these aggregates spread from one brain region to the next. Are some regions selectively vulnerable? Are others resilient? These questions will need to be answered across space and time. Several recent studies have applied computational network diffusion models to predict the early stages of α-syn spreading patterns (Henderson et al., 2019a; Pandya et al., 2019), providing evidence that anatomical connectivity can accurately predict these patterns. Such spatiotemporal models will be crucial for further understanding and ultimately treating a progressive disease like PD at variable points of its progression. Recent studies have also used transgenic animal models and cell-specific labeling techniques to explore the genetic determinants behind α-syn spreading (Henderson et al., 2020; Henrich et al., 2020), indicating that levels of gene expression, such as endogenous α-syn or GBA1, are additionally important factors. However, previous studies did not quantify full three-dimensional whole-brain pathology and did not report spreading patterns beyond 6 months post-injection. We hypothesized that both a full spatial representation and tracking the later stages of spreading would be crucial in characterizing changes in pathology and degeneration that are known to occur in such progressive neurodegenerative diseases.

Here, we used tissue clearing and light-sheet fluorescence microscopy to three-dimensionally image α-syn pathology in the whole mouse brain, as well as a computational pipeline for anatomically mapping each neuronal inclusion to the Allen Reference Atlas (ARA). Merging the data into the ARA coordinate system allowed comparisons with previous studies that mapped mesoscale axonal projections between neuroanatomical regions (Oh et al., 2014) and spatial transcriptomics across many genes (Lein et al., 2007). Statistical comparisons of brain maps at various stages of disease progression revealed a biphasic spreading and decay curve with differential timing by region. Furthermore, tracking the size of each α-syn aggregate longitudinally across regions uncovered a pattern of steady increase and rapid decline of mean aggregate size per region, implying both prion-like aggregation and subsequent neurodegeneration. In order to capture these simultaneous effects, we developed a computational model that incorporates spreading, aggregation, decay, and spatial gene expression of pathology across the whole brain. The development and validation of this model provides a foundation for tracking both the origin and progression of this highly complex disease.

### Results

### Whole-Brain Quantification of α-Synuclein Pathology Using Tissue Clearing and Light-Sheet Microscopy

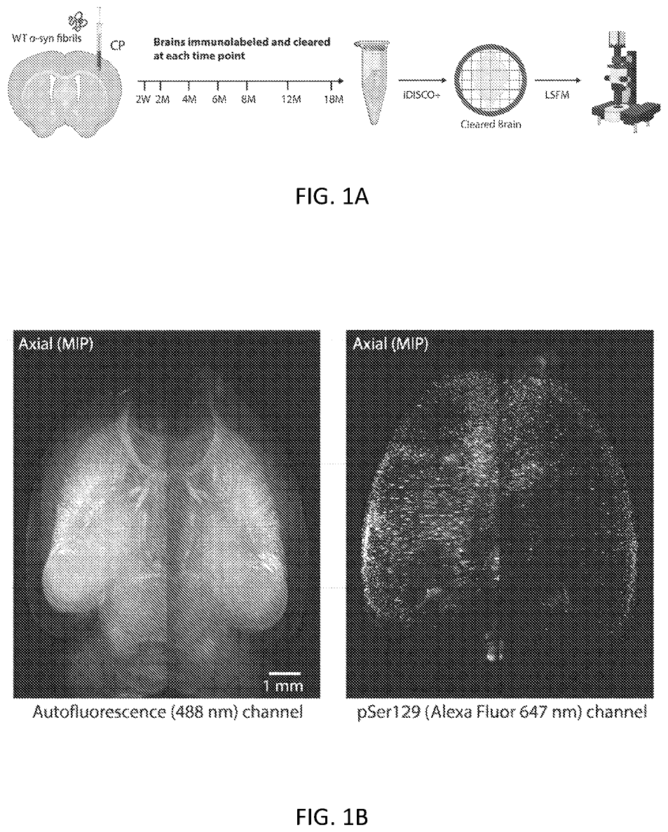

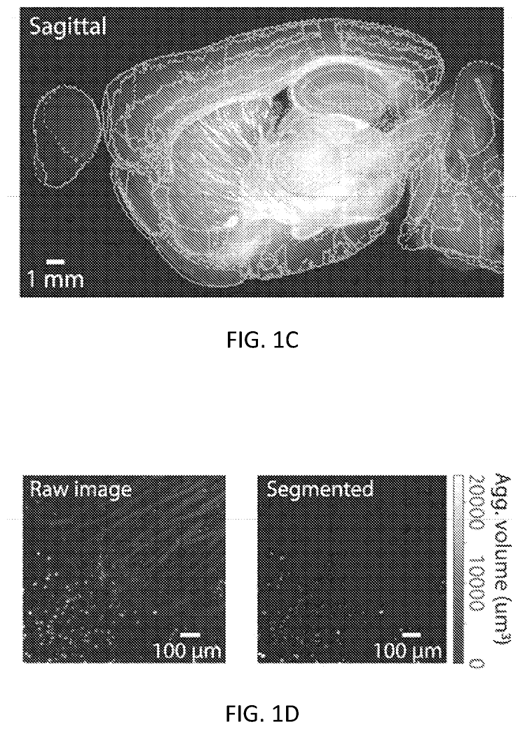

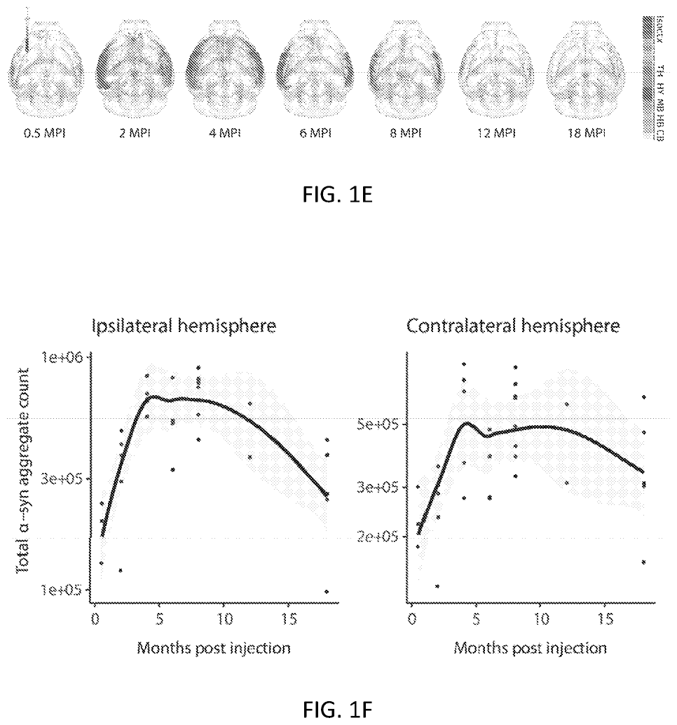

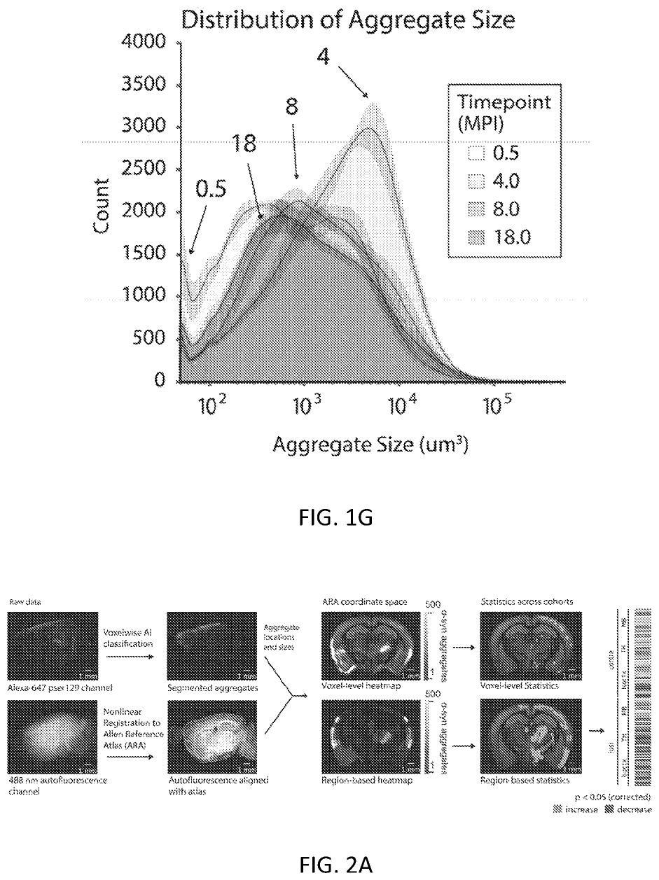

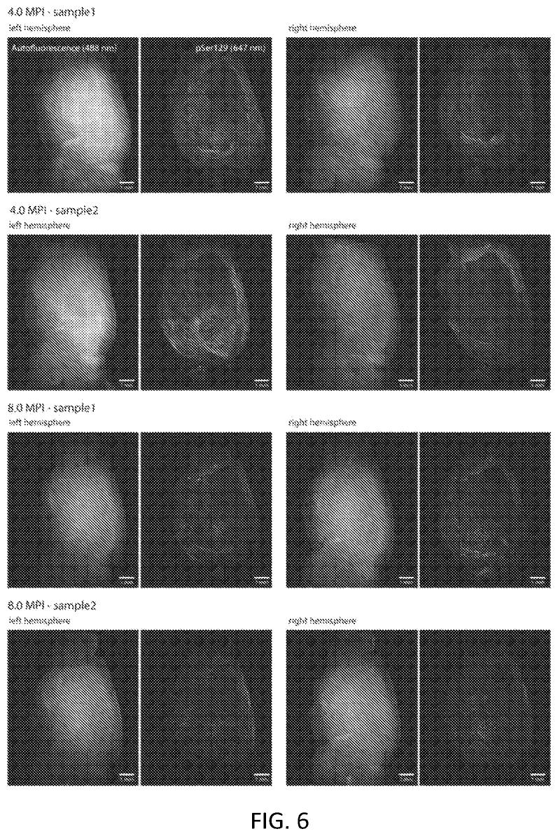

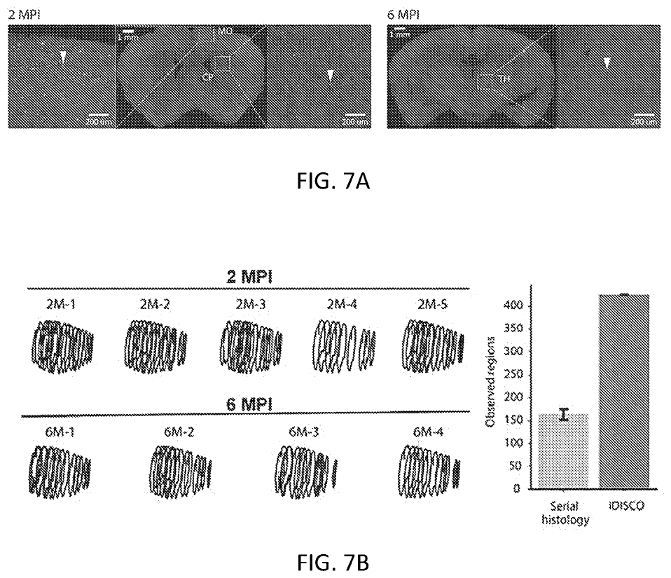

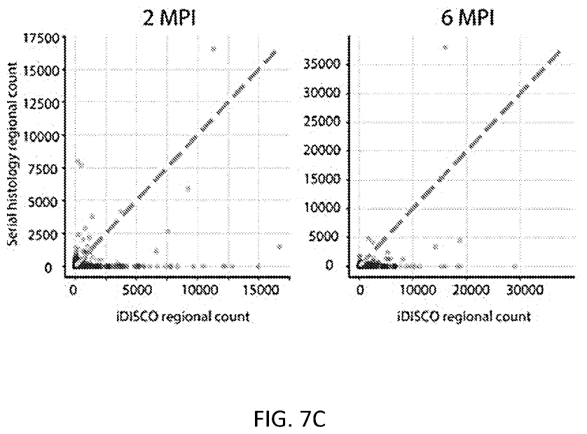

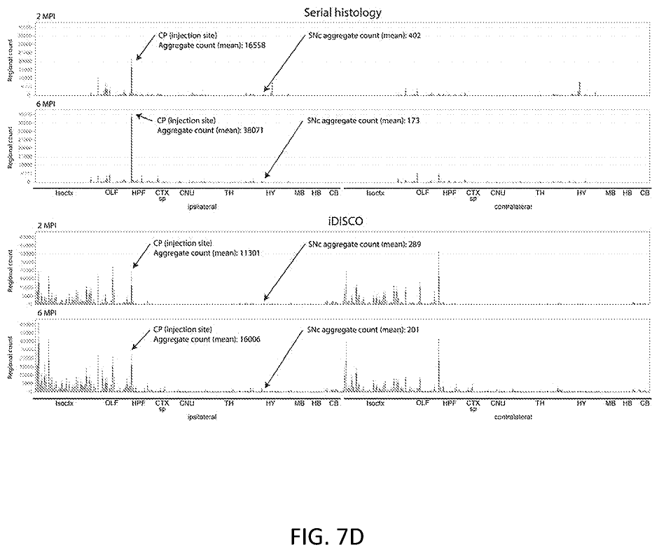

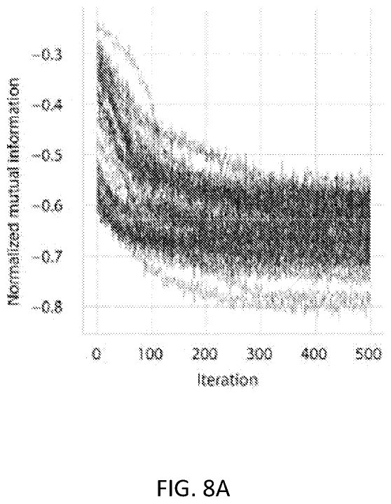

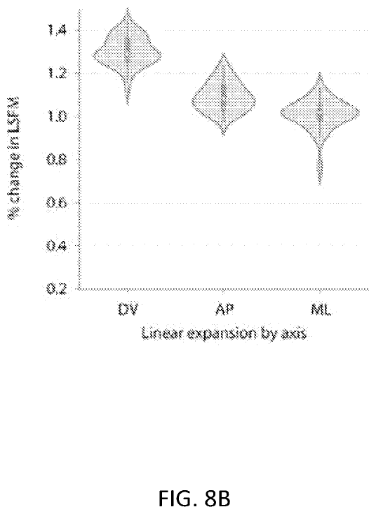

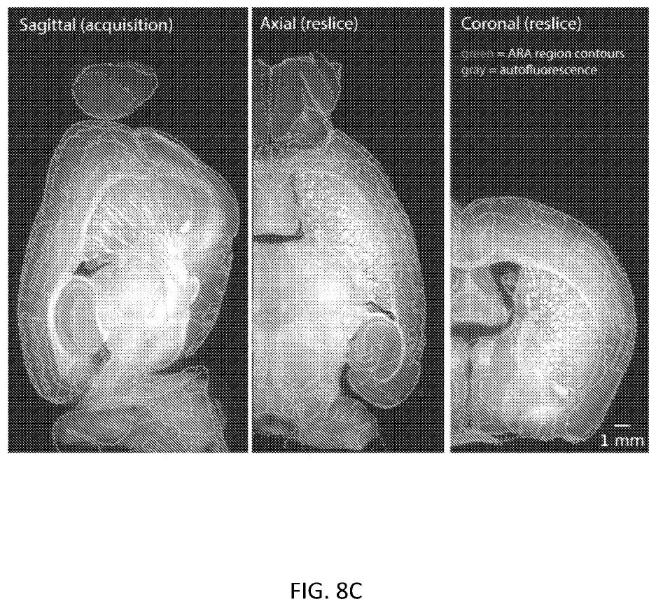

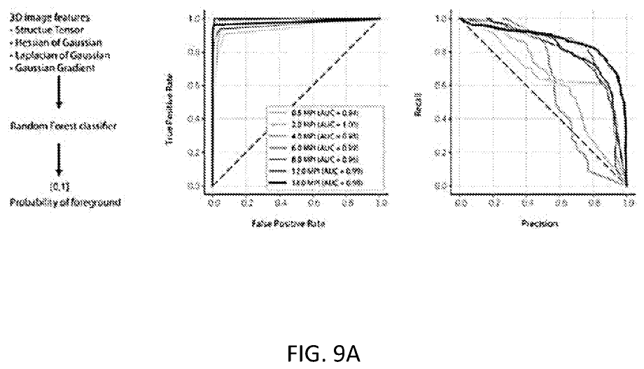

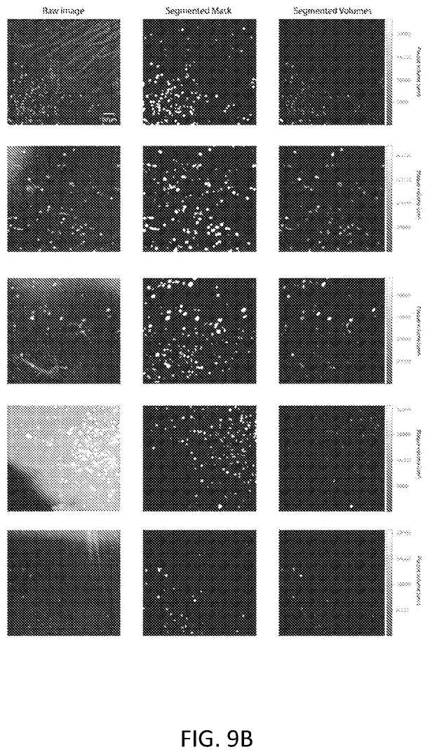

The direct injection of α-syn pre-formed fibrils (PFF) triggers whole-brain pathology and neurodegeneration, serving as a robust model of Parkinson's disease (Henderson et al., 2019b; Luk et al., 2012a). To track pathology throughout disease progression, we ipsilaterally injected mice aged 8-10 weeks with α-syn PFFs in the striatum, or Caudoputamen (CP), and processed mouse brains for immunohistochemistry at various timepoints up to 18 months post injection. (FIG. 1A). Building off of recent advancements in three-dimensional whole-brain immunolabeling and imaging, we optimized the iDISCO+ protocol to immunolabel α-syn aggregates (using an antibody to specifically detect aggregated endogenous α-syn phosphorylated on Serine 129) (Renier et al., 2016; Renier et al., 2014) and imaged samples using a light-sheet microscope (FIGS. 1B and 6). We validated virtual sections from these three-dimensional datasets against traditional serial histology (FIG. 7) and found them to be consistent with previous studies that used the same injection site (Bieri et al., 2019; Henderson et al., 2019a; Luk et al., 2012a), with pathology developing in both the ipsilateral striatum, intermediate layers of the cortex, and substantia nigra at both 2 and 6 months post-injection (FIG. S7A). After imaging, we used a quantification pipeline building on several open source software tools, such as Ilastik (Berg et al., 2019) (machine learning library) and ClearMap (Renier et al., 2016) (registration library), to detect each α-syn aggregate and assign it to a voxel or anatomical region from the Allen Reference Atlas (FIGS. 1C, 1D, 8, and 9) (Oh et al., 2014). Since we were able to capture each α-syn neuronal inclusion's three-dimensional volume (FIG. 1D), we could visualize the distribution of aggregates and aggregate size across timepoints (FIG. 1E). Plotting the total α-syn aggregate count in the whole brain against timepoints of months post injection (MPI) revealed a biphasic curve (FIG. 1F), starting with pathological spreading (between 0-6 months post injection) followed by decay (between 8-18 months post injection). We observed a relatively larger spike of smaller aggregates at the earliest time point post injection, whereas at 4- and 8-months post injection the distribution of volumes shifted to double the mean aggregate-volume per voxel. Surprisingly, at the latest 18-month time point, this distribution shifted back to lower aggregate burden, which was not accompanied with the presence or reappearance of smaller size aggregates (FIG. 1G).

### Statistical Analysis Across Longitudinal Groups Reveals Region-Dependent Spreading, Accumulation, and Decay.

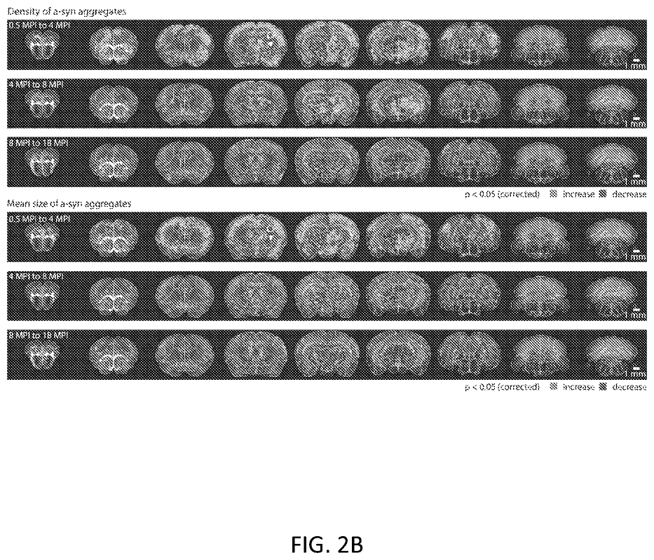

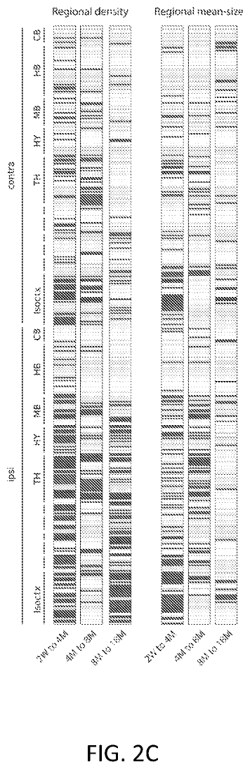

To define the spatiotemporal patterns of α-syn pathology, we ran statistical comparisons at the voxel and regional levels between cohorts at various times sacrificed post-injection. Because of variability in spreading patterns across adjacent timepoints, we instead used timepoints spaced at least 3 months apart for these comparisons. Our quantitative pipeline captured both total aggregate count and mean aggregate volume for each voxel (FIG. 2A). Therefore, we performed statistical comparisons for each of these two metrics between each selected pair of timepoints. Comparing total aggregate count at the voxel level (FIG. 2B), we observed statistically significant clusters with widely varying rates of both spread and decay for different brain subregions. For example, when widespread pathology in the cortex has already aggregated and begins decaying by 4 months post-injection, aggregates begin to appear for the first time in various subcortical clusters, including within the thalamus and contralateral hippocampus. Statistics comparing the mean aggregate size at the voxel level demonstrate a similar initial increase both cortically and subcortically from 0.5 to 4 MPI, followed by a whole-brain decrease from 4 to 8 MPI (FIG. 2B). The clusters of significant increase or decrease from the voxel-level analysis generally obeyed the boundaries of anatomical brain regions; we observed similar biphasic trends when computing both the α-syn aggregate count and mean-size metrics across regions from the Allen Brain Atlas (FIG. 2C). Thus, different brain regions exhibit different spatiotemporal patterns/dynamics of α-syn spreading, accumulation, and decay.

### Computational Model of Spreading

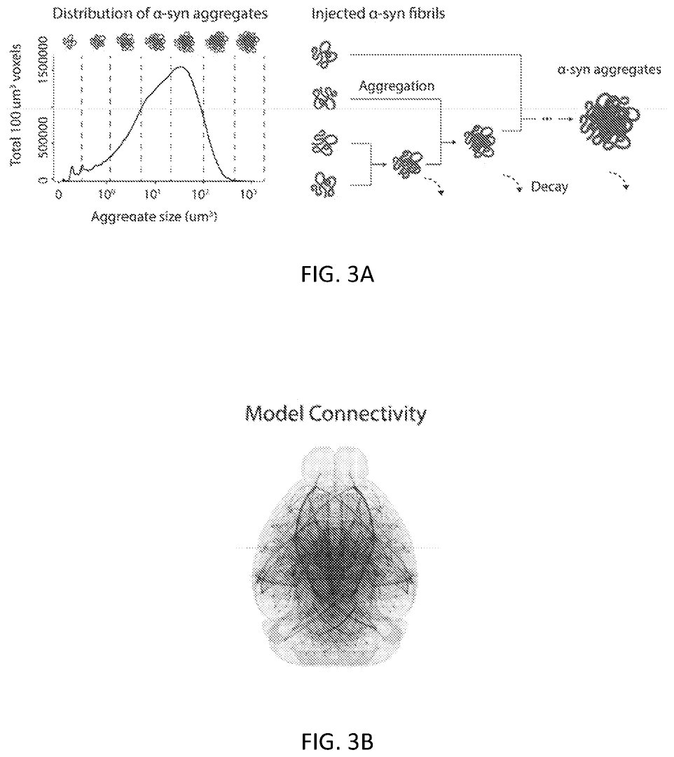

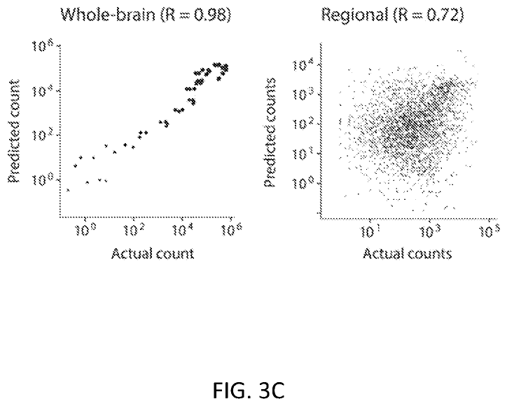

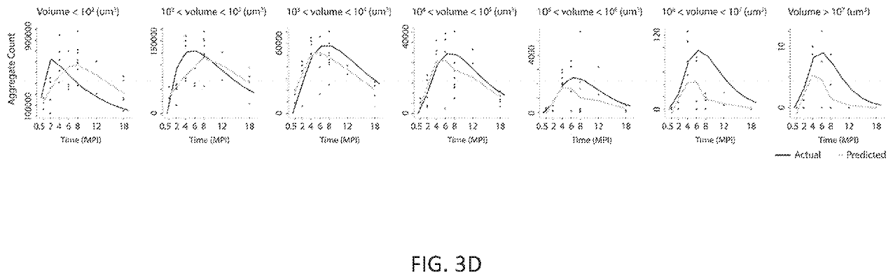

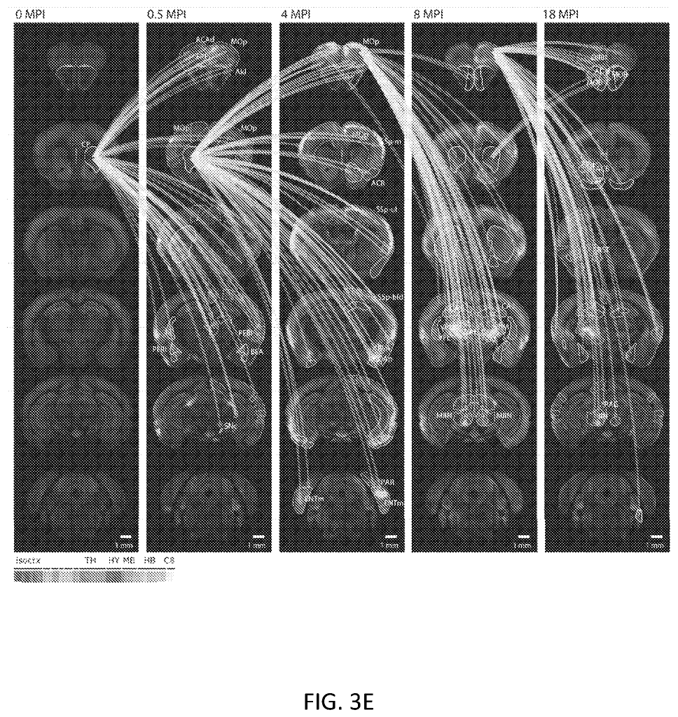

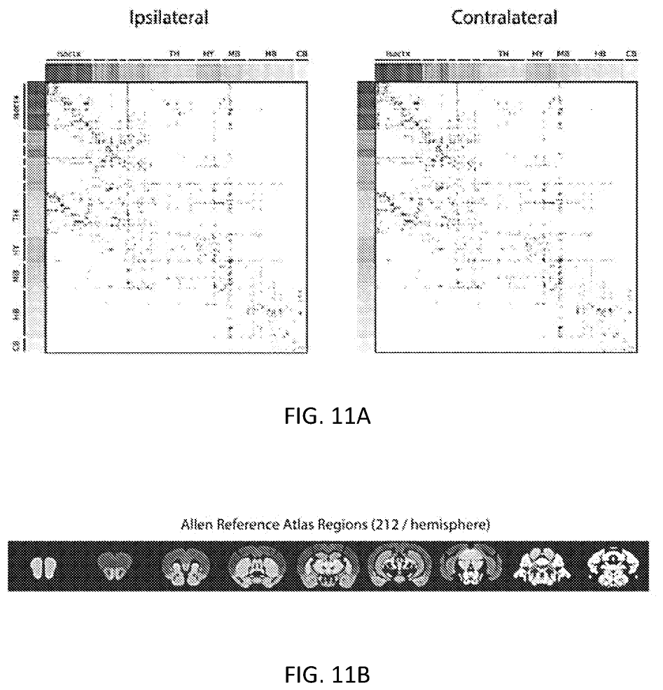

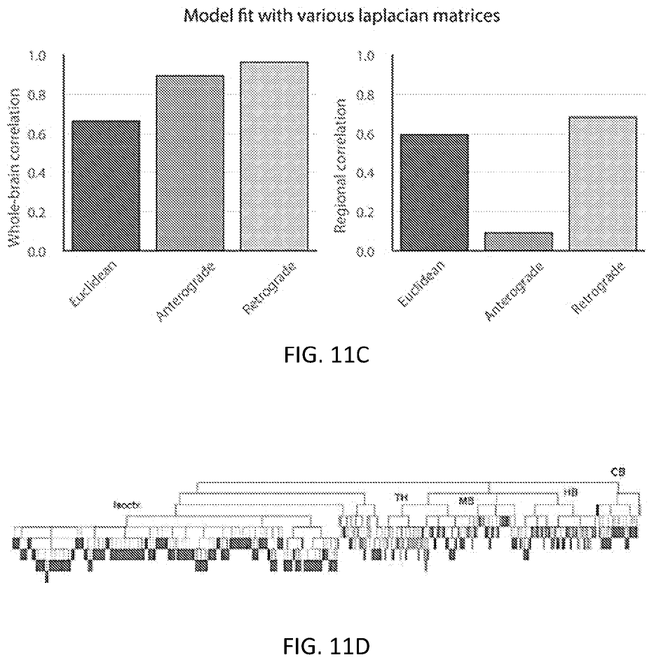

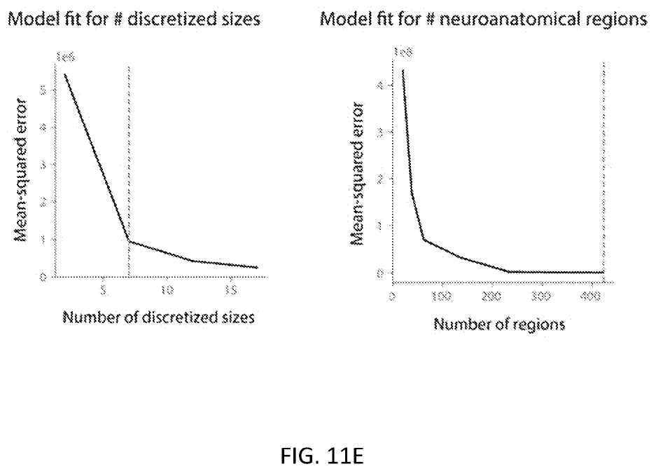

Despite being able to explain much of the initial regional variation in spreading, previous applications of computational models (Henderson et al., 2019a) only accounted for the spreading of α-syn. Thus, they are unable to generalize to later time points throughout the disease, notably even at 4 MPI where decay in pathology starts to occur (FIG. 2B). To more accurately predict the progression of α-syn pathology, we developed a new computational model by incorporating mechanistic insights regarding α-syn pathogenesis and trafficking from recent in vivo and in vitro studies. The key steps in this model consist of α-syn uptake into neurons, intracellular processing and interactions, and finally release of pathological α-syn. A set of differential equations model a discretized distribution of α-syn aggregate counts in each neuroanatomical region (FIG. 3A). The model initially assumes quick uptake of injected α-syn fibrils into neurons within the target region. This has been confirmed by studies showing that extracellular α-syn fibrils are integrated into neurons through endocytosis (Brahic et al., 2016; Desplats et al., 2009; Henderson et al., 2019b; Konno et al., 2012). These injected fibrils are considered the smallest discrete pathological unit that can exist in the brain. However, as misfolded α-syn is processed through endo-lysosomal and cytoplasmic compartments, it can both recruit endogenous α-syn into a pathogenic fibrillar state, as well as merge with existing fibrils to form aggregates of larger size. Building off previous studies (Bieri et al., 2019), we assume retrograde spreading of any α-syn aggregate through the brain connectome, which the model incorporates as diffusion through a directed weighted graph (FIGS. 3B and S11A). We derived this anatomical connectivity from the Allen Connectivity Atlas (Oh et al., 2014), which includes 424 regions across the whole brain (FIGS. 11A and 11B; Table S1). Our model does not incorporate fragmentation of α-syn aggregates inside of the cytoplasm because previous studies have shown the fragmentation rate to be undetectably low (Gaspar et al., 2017).

Although we set most model parameters a priori, we fit the model's parameters controlling the rate of spread and decay to data from α-syn PFF injection into the striatum, ranging from 0.5 to 18 MPI. Despite only being fit to maximize the model's output in predicting the whole-brain α-syn aggregate count (FIGS. 3C and 3D), the resultant model additionally captures the regional variation in pathology with a high Pearson correlation coefficient of 0.72 (FIG. 3C), providing evidence for the theory of a primarily retrograde neuronal spreading mechanism. We tested additional networks based on either anterograde connectivity or Euclidean distance between regions but neither could capture both whole-brain and regional variability (FIG. 11C). Calculating model sensitivity through the Jacobian matrix allows for weighting of brain pathways that account for the most significant α-syn spread. This analysis highlights many retrograde pathways in the cortico-basal-ganglia-thalamo-cortical loop. Several examples with the highest Jacobian weight are early spreading from ipsilateral striatum to many cortical areas, such as the infralimbic area (ILA), main olfactory bulb (MOB), and gustatory area (GU). This is followed by spreading from the cortex to thalamus and other subcortical areas at later time points (FIG. 3E).

### Prediction of Spreading Patterns for Different Injection Sites

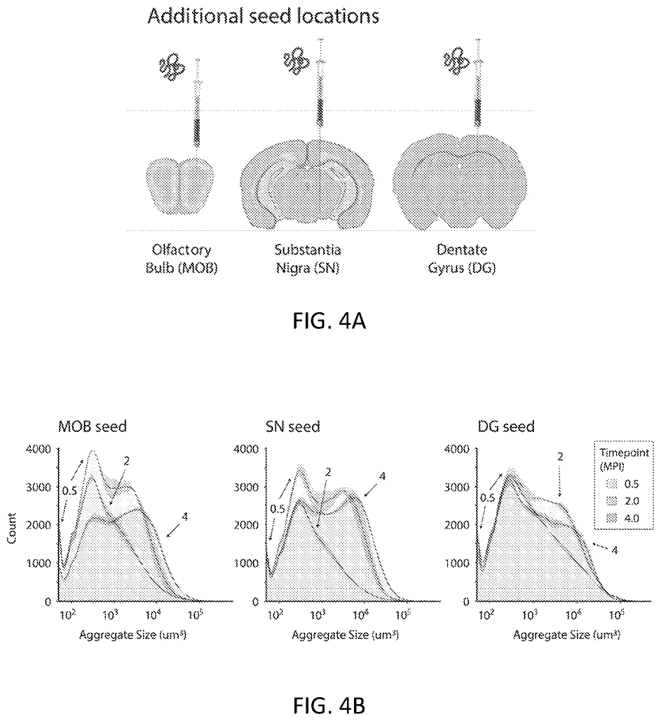

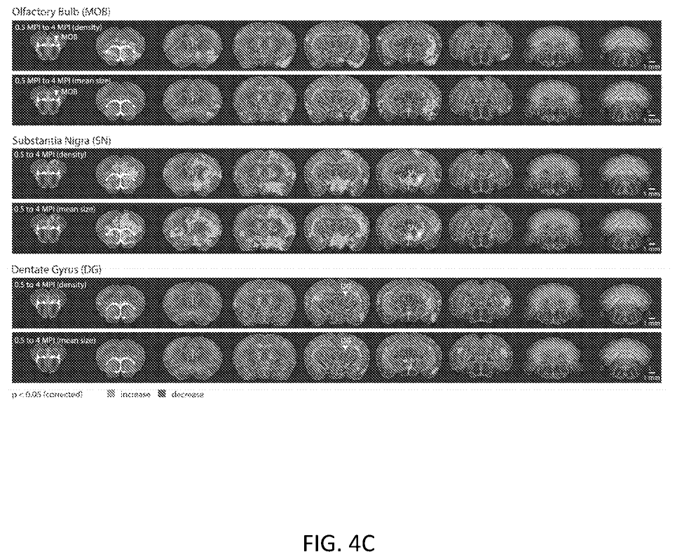

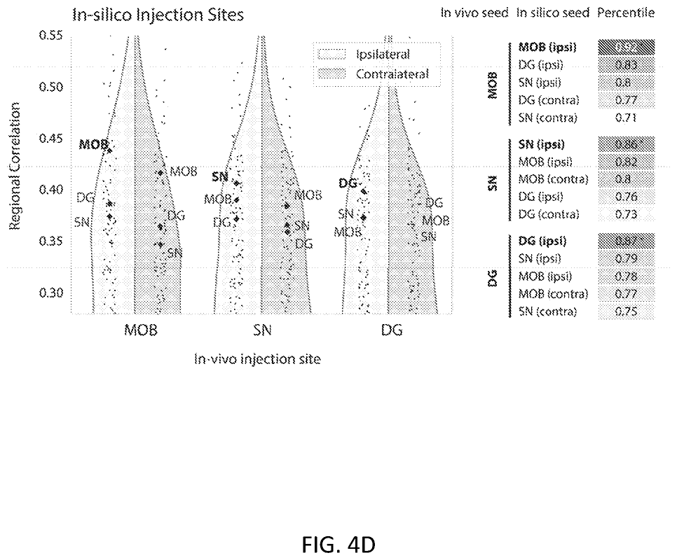

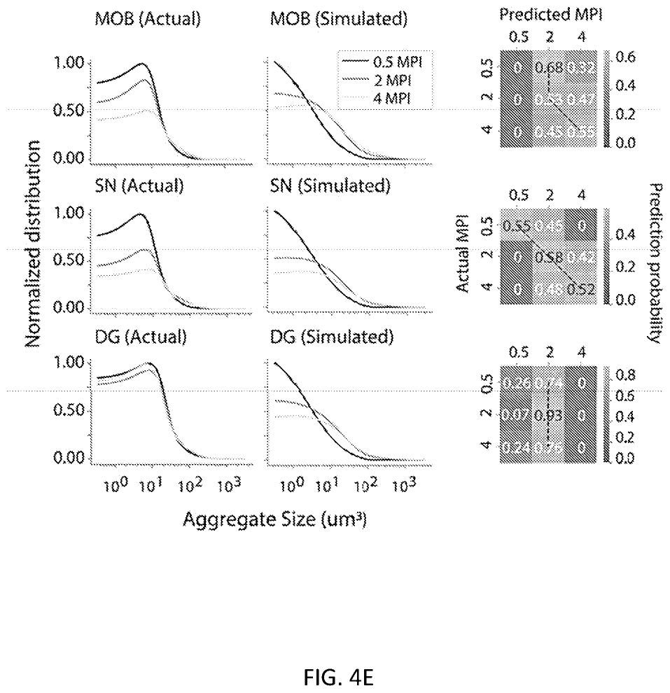

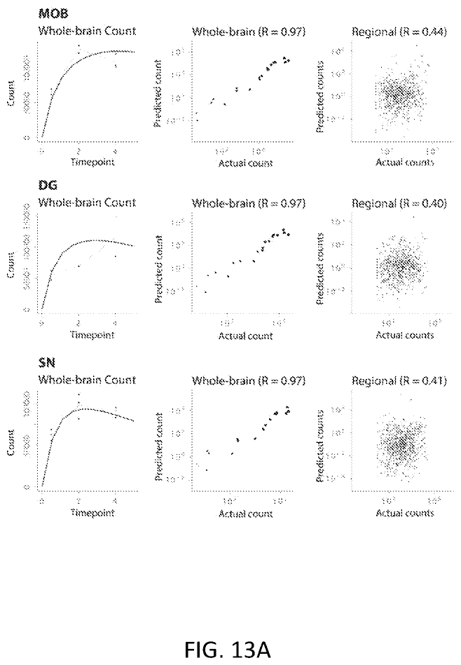

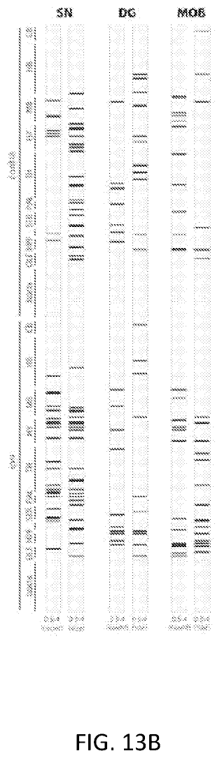

After finding that our model based primarily on anatomical connectivity was able to accurately predict pathology resulting from α-syn PFFs injected into the striatum, we next tested the generalizability of this model to different seed locations (i.e., injection sites). We performed additional injections of α-syn PFFs throughout various regions of the brain and compared our model's predictions with actual quantified pathological states. Since one of the prevailing theories behind α-synucleinopathies is that a single seeding event can result in spread throughout the nervous system, we chose a variety of distinct seed locations with relevance for Parkinson's disease and other synucleinopathies: substantia nigra pars compacta, main olfactory bulb, and dentate gyrus (FIG. 4A). Using our iDISCO immunolabeling, imaging, and computational processing pipeline, we quantified voxel-level and regional aggregate density and mean-size for brains at 0.5 MPI, 2 MPI, and 4 MPI for each seed location. These seed locations induced remarkably consistent distributions of aggregate size (FIG. 4B), with the earliest 0.5 MPI maps displaying spikes of small aggregates, and this distribution tending towards larger aggregates over time. However, statistical tests from 0.5 to 4 MPI, at both the voxel and neuroanatomical level, yielded distinct spatial patterns of spreading and aggregation depending on the seed location (FIGS. 4C and 13B).

We then applied our computational model to predict the regional pathological density for each discretized size bin across time. This involved modifying the initial condition of the simulated model and integrating forward in time, while keeping all parameters and hyperparameter values fixed. After iterating through each region in silico and generating the full time series of disease progression, we selected the region that best predicts the pathological state for a given dataset (FIG. 4E). This method consistently predicted the correct ipsilateral hemisphere and neuroanatomical region of the initial seed from amongst all other seed locations (FIG. 4E). Since all additional seed locations demonstrated consistent distributions of aggregate volume across the whole brain (FIG. 4B), we hypothesized that we could also predict the duration of time since injection through inversion of this model, which we indeed found to be the case (FIG. 4F).

Encoding Spatial Transcriptomics into the Regional Model

Consistent with current models (Henderson et al., 2019a), anatomical connectivity seems to be the primary driver of the patterns of α-syn spread; our computational model with edges simply weighted by anatomical retrograde connectivity was able to predict the regional spreading patterns with a high degree of correlation. But Parkinson's disease is associated with a diverse set of genetic susceptibility factors. Do these converge on and impact α-syn spreading? The gene encoding α-syn itself, Snca, directly impacts spreading, because knockout of α-syn expression in mouse is sufficient to prevent widespread pathology following injections of α-syn PFFs, almost certainly because there is no endogenous α-syn to convert into aggregated form (Luk et al., 2012a; Luna et al., 2018; Taguchi et al., 2014). Other PD genes have also been connected to α-syn spreading. Transgenic mice engineered to express a PD-causing mutation in the Lrrk2 gene (LRRK2:G2019S) showed increased α-syn aggregation upon PFF injection (Bieri et al., 2019; Henderson et al., 2019a), as did human iPS neurons (Bieri et al., 2019).

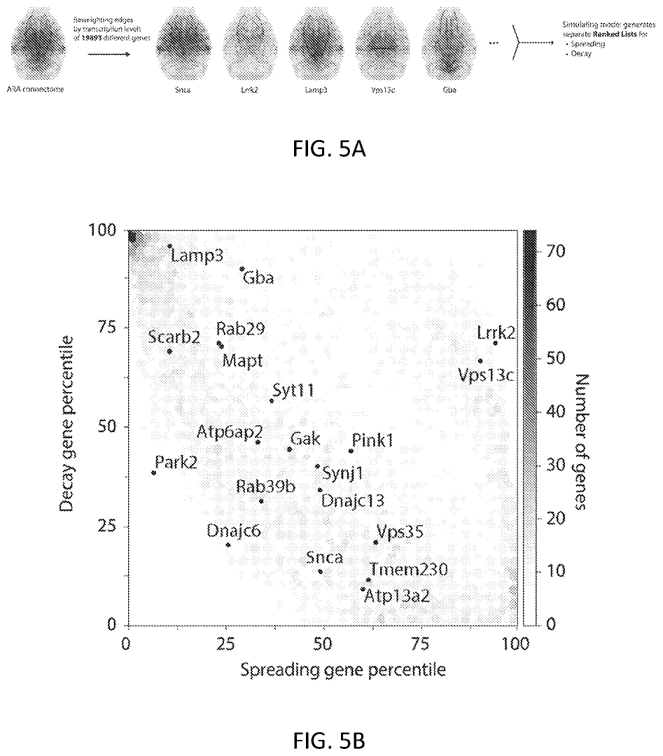

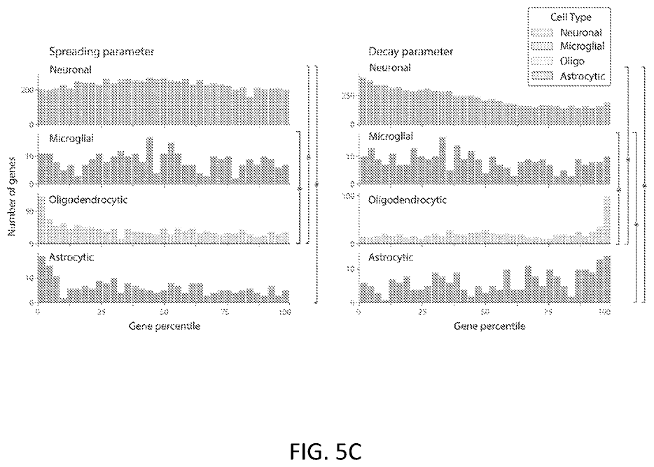

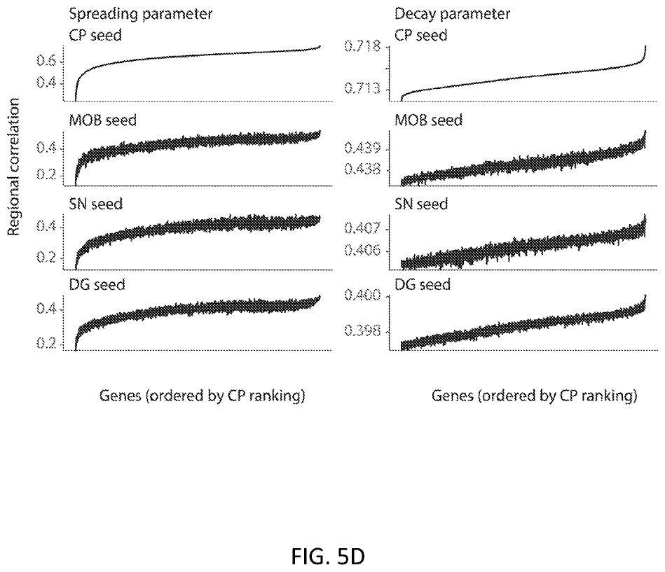

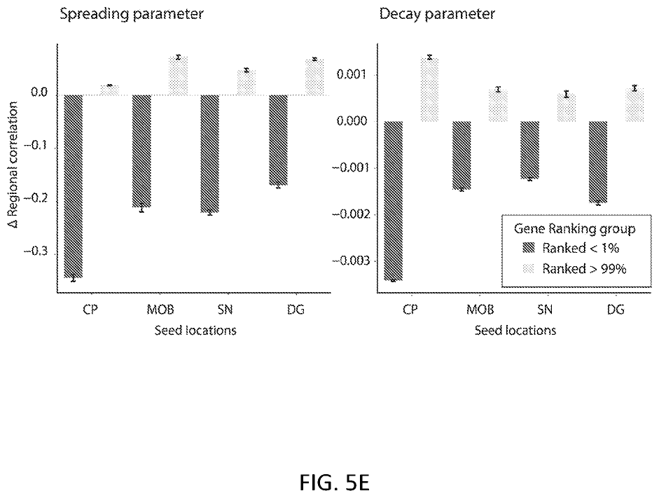

We hypothesized that encoding regional genetic data into the computational model would allow us to rank a gene's effects on the separate spreading and decaying steps of the model and potentially improve the model's predictive power, as measured by the Pearson correlation coefficient of actual versus predicted regional variation. We encoded 19,893 regional gene density maps from the Allen in situ hybridization (ISH) database (Lein et al., 2007) into our previously fit computational model, using the same 424 regions spanning the whole brain (FIG. 5A). Re-simulating α-syn progression with each encoded gene density map provided a distribution of their effects on the spreading and decay parameters (FIG. 5B). Interestingly, the Lrrk2 gene improved the model's regional predictions when incorporated into the spreading parameter, and in that case ranked very highly (94^{th} percentile) amongst all genes. This is consistent with the hypothesis that Lrrk2 is important in vesicular trafficking pathways (Henderson et al., 2019b), and the recent evidence that reducing levels of Lrrk2 decreases α-syn aggregation (Bieri et al., 2019).

After associating each gene with its most likely cell type using the Allen Cell Types RNA-Seq Database (Tasic et al., 2018), we explored the relationship between a gene's cell type and its ranking. Since our model assumes a neuronal mechanism of transport, we hypothesized that genes from neuronal cell types would dominate the model's spreading term, which is indeed the case (FIG. 5C). However, for the decaying term in the model, we unexpectedly found a cluster of genes primarily expressed in oligodendrocytes, with Myelin basic protein (Mbp) being the highest ranked gene (FIG. 5C). This finding is consistent with new data integrating genome-wide association studies with cell type atlases to show that oligodendrocytes play a key role in Parkinson's disease (Bryois et al., 2020). Furthermore, accruing evidence suggests that another synucleinopathy, MSA (multiple system atrophy), an aggressive degenerative disease characterized by oligodendroglial cytoplasmic α-syn inclusions, behaves like a prion and that α-syn may indeed be the prion (Prusiner et al., 2015). Moreover, recent research suggests that the cytoplasmic milieu of oligodendrocytes promotes the formation of particularly potent α-syn seeds, which can spread to neurons (Peng et al., 2018). The enrichment of oligodendrocyte genes in our model (FIG. 5C) lends further support for a role of oligodendrocytes in α-syn spreading.

Lastly, we found that using other seed locations to re-simulate the model and evaluate gene importance generated remarkably consistent rankings independent of injection site-based dataset (FIG. 5D). Applying the average of the top percentile of ranked genes improved this correlation value for all injection sites, while conversely, the average of the lowest percentile of genes drastically reduced the predictive power of the model (FIG. 5E). Future studies will be required to define the functional impact of these top-ranked genes on α-syn spreading but this list provides a resource for testing hypotheses.

### Discussion

Here, we demonstrate the ability of a mesoscale computational model to predict both the origin and progression of a neurodegenerative disease by using this computational model alongside quantitative high-resolution, whole-brain imaging. As longitudinal statistics across neuroanatomical regions and at the voxel-level demonstrated, the spreading and decay of pathology following seeding of α-syn PFFs is highly dynamic in nature, with many regions containing overlapping phases of spreading and decline. Nonetheless, our computational model is based on known mechanisms of α-syn PFF pathogenesis and accurately reconstructs the longitudinal counts of α-syn aggregates of various sizes across 424 brain regions. Retrograde anatomical connectivity can explain much of the regional variability in spread, but separately encoding 19,893 genes from a spatial transcriptomics database into the model additionally allowed us to uncover the relative involvement of genes in the model's spreading and decaying terms.

Although the incorporation of regional gene information into the models only uncovers correlative relationships between genes and the spreading and decaying of α-syn pathology, we provide evidence that the top-ranked genes in this analysis generalize to other PFF seed locations as well. With this list of genes in hand, future studies will aim to test their functional impact on α-syn spread and some may even represent therapeutic targets to slow down or stop spread.

Idiopathic PD, which represents most cases, can be seeded from various parts of both the nervous system and peripheral organs (Challis et al., 2020; Kim et al., 2019; Peelaerts et al., 2015; Sacino et al., 2014). The comparisons between in silico simulation of α-syn pathogenesis and data from various injection sites provide a testbed for this model and demonstrate its generalizability in predicting the origins and future patterns for arbitrary seeding datasets. In a broader sense, this model holds promise for analyzing human brain imaging data (such as once accurate α-syn PET ligands are developed) to wind the clock back and predict how and where α-syn pathology originated. The clock can also be wound forward to predict the future trajectory and tailor therapeutic interventions accordingly. We propose this clinical application of such a model as relevant to PD or any progressive protein-spreading neurodegenerative disorder, such as Alzheimer's disease, amyotrophic lateral sclerosis, or frontotemporal dementia (Goedert et al., 2010; Guo and Lee, 2014; Jucker and Walker, 2013). Because our model provides a metric for predicting the in vivo seed location when given an unseen set of pathological states, being able to predict the seed location and progression given a pathological state would have high utility in clinical diagnostic and therapeutic applications for many of these neurodegenerative diseases.

We found that encoding gene transcription levels by neuroanatomical region into our model significantly impacted fitting to the data. This potentially points to the implications of relative gene transcription levels in the spreading and decay process. This comprehensive modeling and gene encoding approach is what enables this ability to rank genes. In terms of detailed implications, future validation is needed to explore certain genes of interest, but the main point is that we can start to form a hypothesis from these gene rankings. We also highlight the significant finding in this study that this process produced similar rankings of genes across datasets from different pre-formed fibrils seeding locations (FIGS. 5D and 5E), even with these seeding locations producing vastly distinct spreading patterns of pathology (FIG. 4C). This consistency supports the unique information that this approach provides, and we believe will enable future studies to explore the roles of these genes further.

Future extensions to this model could include considering additional mechanisms such as vascular, ventricular, or glial densities. Many studies have also shown spreading of seeds from both the gut and peripheral nervous system. However, owing to the current lack of quantitative atlases that connect the peripheral-to-brain or gut-brain axes, and the technological difficulty in imaging a cleared whole mouse body using light sheet microscopy, we only focused on the dynamics of pathology across the central nervous system. However, our model could integrate these datasets when they become available. Furthermore, although the encoding of genes from a spatial transcriptomics database revealed potential implications of these genes in either the spreading or decay of pathology, this analysis does not take into consideration protein expression levels, or any effects caused by genetic mutation. This computational model could help form hypotheses for further studies looking into these effects.

As our labeling approach with DISCO utilizes polyclonal secondary antibodies, we expect that multiple polyclonal secondary antibodies bind to each primary antibody, thus providing fluorescent signal amplification. An inherent limitation in this approach is that an alpha-synuclein aggregate's size, as measured by the three-dimensional morphology in the fluorescence channel, is not directly indicative of the actual aggregate size (nonlinear but monotonic). However, we expect this multiple binding to affect all samples equally. Given that we are performing large-scale and stringent statistics across many cohorts of mice, we expect that our statistical tests can still uncover regions of interest that show significant increase or decrease in aggregate size.

The clinical detection of pathological α-syn and other proteinaceous seeds for neurodegenerative diseases is currently performed primarily through post-mortem analysis. However, many advancements in nuclear medicine, similar to PET and SPECT radiotracers that detect amyloid depositions in vivo, will likely allow for quantitative evaluation of a patient's pathological state. Current tracers work well for amyloid-beta and tau but are still in development for α-syn. Given the various etiologies that have been observed clinically, it will undoubtedly be essential to be able to differentiate between synucleinopathies with different origins and trajectories. The generalizability and interpretability of the computational model we present here offers unique advantages because it can both infer the progression of α-syn spreading patterns when given the current pathological state, or inversely produce the likely seed locations, and time since seeding, that led to this state. All these applications will help empower more accurate disease classification and prediction of clinical phenotypes for a wide array neurodegenerative disease.

### Limitations of the Study

The methods presented in this study have demonstrated that whole-brain imaging and computational modeling can accurately describe and predict the longitudinal dynamics of pathology in neurodegenerative disease. While this model can accurately reconstruct the observed dynamics of whole brain pathology change over time across 424 neuroanatomical regions, it made several important assumptions based on recent discoveries in the literature. Specifically, these include neuronal uptake of injected α-syn fibrils (Brahic et al., 2016; Desplats et al., 2009; Henderson et al., 2019b; Konno et al., 2012), synaptic spreading of α-syn pathology (Bieri et al., 2019), prion-like aggregation of pathology into larger units, and eventual decay of this pathology (Luk et al., 2012a). Future work could extend the model to incorporate parameters that were not actively considered and test the sensitivity of the model's predictions to these assumptions. For example, it is possible that grouping aggregate counts into the 424 neuroanatomical regions does not fully capturing the complex pathology dynamics we observed. Although we tested multiple sets of neuroanatomical regions across the atlas hierarchy of regions (FIG. 13D), this number was ultimately constrained by the Allen Connectivity Atlas (Oh et al., 2014). Future studies could take full advantage of the high-resolution images obtained in our study to create more detailed models that take pathology dynamics across cortical layers or more granular anatomical regions into account.

### Experimental Model and Subject Details

### Animals

Mouse husbandry and procedures were performed in accordance with institutional guidelines and approved by the Stanford Administrative Panel on Animal Care (APLAC). 10-12-week-old male C57Bl6J mice (The Jackson Laboratory, cat #000664) were used for stereotaxic injections. Mice were housed under specific pathogen-free conditions under a 12 h light-dark cycle, ad libitum diet and free access to water.

### Method Details

### PFF Preparation

The expression and purification of mouse wild-type α-syn was performed as previously described (Ghee et al., 2005). α-syn fibril formation was induced by incubation in 50 mM Tris-HCl, pH 7.5, 150 mM KCl buffer at 37° C. under continuous shaking in an Eppendorf Thermomixer at 600 rpm. α-syn fibrils were centrifuged twice at 15,000 g for 10 min and resuspended in PBS. All fibrils were fragmented prior to in vivo use by sonication for 20 min in 2-ml Eppendorf tubes in a Vial Tweeter powered by an ultrasonic processor UIS250v (250 W, 2.4 kHz; Hielscher Ultrasonic, Teltow, Germany). 5 ug of fibrils/mouse were used for in vivo mouse experiments. The fibrils were endotoxin free, as assessed using the Pierce LAL Chromogenic Endotoxin Quantification Kit.

### Fibril Injections

Stereotaxic injections were performed on 10-12-week-old adult mice. Animals were placed in a stereotaxic frame and anesthetized with 2% isoflurane (2 L/min oxygen flow rate) delivered through an anesthesia nose cone. Ophthalmic eye ointment was applied to prevent desiccation of the cornea during surgery. The area around the incision was trimmed, cleaned, and disinfected. A small hole was drilled above the injection site. PFF or vehicle solutions were injected unilaterally into the dorsal striatum, hippocampus, olfactory or substantia nigra using the following coordinates (from bregma): Striatum—anterior (AP)=+0.4 mm, lateral (ML)=+/−1.85 mm from midline, depth (DV)=−2.7 mm (from dura). Olfactory bulb: AP, +4.50 mm; ML −0.75 mm; DV −1 mm. Dentate gyrus: AP −2 mm, ML=1.5 mm, DV=−2.1 mm. Substantia nigra pars compacta: AP −3.1 mm, ML 1.2 mm, DV −3.75 mm. Mice were injected with sonicated PFFs (5 μg/mouse) or PBS vehicle control. PFFs were sonicated prior to injection. 1 μl volume was injected at a rate of 100 nl/min using a 5 μl Hamilton syringe with a 32 G needle. To limit reflux along the injection track, the needle was maintained in situ for five minutes, before being slowly retrieved. The skin was closed with silk suture. Each mouse was injected subcutaneously with analgesics and monitored during recovery. Animals were sacrificed 2 weeks to 18 months post injection.

### Tissue Processing

Mice were anesthetized with isoflurane and transcardially perfused with 0.9% saline followed by 25 ml of 4% PFA. Brains were dissected and post-fixed in 4% paraformaldehyde (PFA) pH 7.4, at 4° C. for 48 hours. Brains for histology stored in 30% sucrose in 1×PBS at 4° C. PFA-fixed brains were sectioned at 35 um (coronal sections) with a cryo-microtome (Leica) and stored in cryoprotective medium (30% glycerol, 30% ethylene glycol) at −20° C. Brains for iDISCO tissue clearing and labeling were stored in PBS with 0.05% sodium azide.

### Tissue Clearing

Each sample was fully immunolabeled and cleared using the previously described iDISCO protocol (Renier et al., 2014), which describes the sample pretreatment, blocking, immunolabeling, and clearing steps in more detail. The methanol pretreatment step was performed for all samples. For primary immunolabeling, an anti-phospho-synuclein (pSer129) Rabbit polyclonal antibody was used at 1:1000 dilution for 7 days, while a Donkey anti-Rabbit IgG (H+L) Alexa Fluor 647 nm antibody was used for secondary immunolabeling at 1:1000 dilution for 7 days. All other clearing parameters were used as previously reported (Renier et al., 2014).

### Immunohistochemistry

Tissue processing and immunohistochemistry was performed on free-floating sections according to standard published techniques. 1:6 to 1:12 series of all coronal sections were used for all histological experiments. Sections were rinsed 3 times in TBST, pre-treated with 0.6% H₂O₂ and 0.1% Triton X-100 and blocked in 5% goat serum in TBST. Free-floating coronal sections were incubated overnight with mouse-α-syn pSer129 antibodies (81A; 1:5000, Covance/BioLegend cat #MMS-5091). After overnight incubation at 4° C., sections were rinsed 3 times in TBST. The primary antibody staining was revealed using fluorescently-labeled secondary antibodies (Thermo Fisher Scientific cat #A-21137). Sections were counter-stained with DAPI, mounted on Superfrost Plus slides (Fisher Scientific) and coverslipped using ProlongDiamond antifade mountant (Thermo Fisher Scientific cat #P36961). Images of pSer129 aggregates were acquired using a Leica DMI6000B inverted fluorescence microscope by an investigator blinded to the treatment group.

### Microscopy

Each sample was imaged using an LaVision Biotec Ultramicroscope II within two days of finishing iDISCO clearing. Microscope settings of a full sheet width, numerical aperture of 0.103, mechanical step-size of 3.5 um, and light-sheet thickness of 7 um were used for all acquisitions. A 488 nm excitation laser and 460/40 nm emission filter (center wavelength/FWHM) were used for each autofluorescence acquisition. A 639 nm excitation laser and 620/60 nm emission filter were used for detecting fluorescent α-syn pathology. The left and right hemispheres of each brain sample were imaged separately. Each acquisition was in the sagittal plane. Each acquired slice had an in-plane resolution of 4.0625×4.0625 um, with a slice resolution of 3.5 um.

### Quantification and Statistical Analysis

### Registration, Segmentation, and Quantification

Upon voxel-wise binarization of the raw iDISCO α-syn fluorescence channel using a machine learning model into foreground (pathology) and background (autofluorescence), binary morphological operations are used to find the connected components. Each connected component is considered a separate α-syn aggregate, and the position (x,y,z), peak intensity (in the corresponding raw data), and volume (number of voxels) of each aggregate is saved.

The non-linear transformation resulting from the registration process is used to transform each aggregate to the Allen Reference Atlas (ARA) coordinate space. Each voxel in this coordinate space is a 100-μm width cube centered at that coordinate. Since the atlas is at a lower spatial resolution (100 μm) than the raw data (4.0625 μm), multiple aggregates may map to a single voxel in the ARA space. For a given ARA voxel, we define the density as the total number of aggregates with centers within that voxel. We also define the total-size for a voxel as the total size of all the aggregates with centers within that voxel. We compute the mean-aggregate-size for each voxel as its total-size divided by the density. A similar calculation is performed for the total and mean intensities at each voxel. The density, mean-aggregate-size, and mean-aggregate-intensity are considered separate metrics.

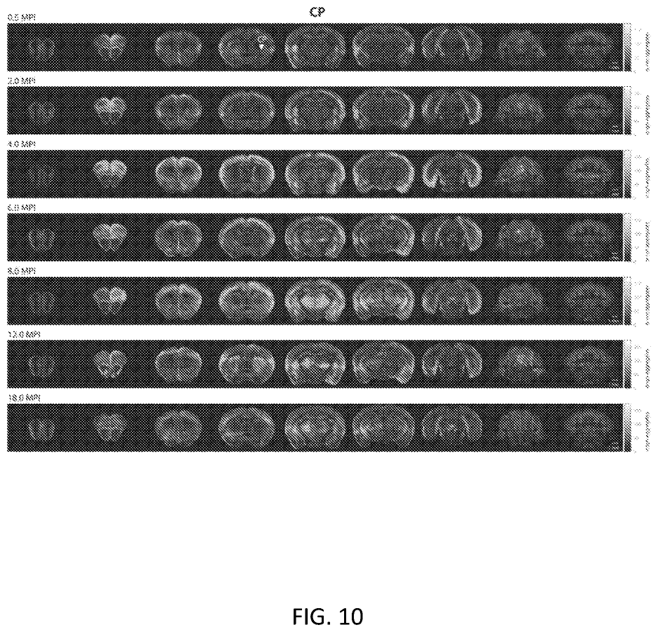

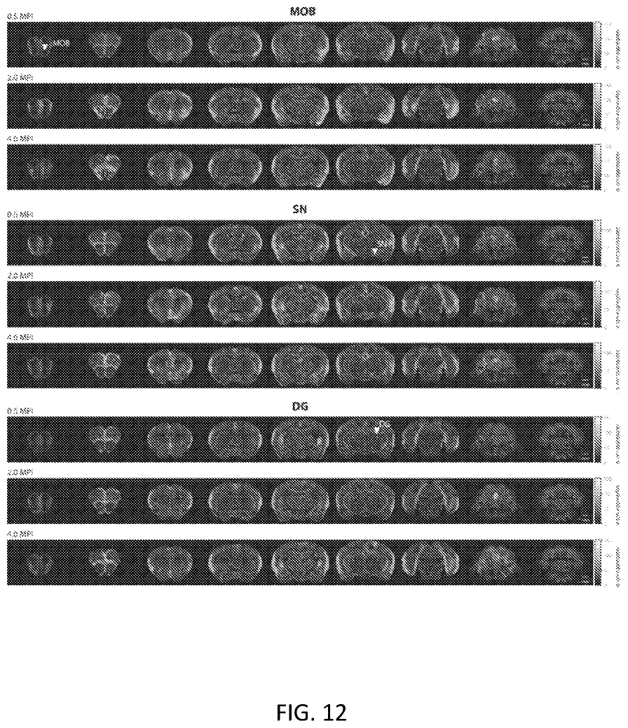

To account for variations in registration quality between samples, a multidimensional Gaussian filter (σ=15 voxels) is applied to the density, mean-aggregate-size, and mean-aggregate-intensity spatial maps to smooth the values across neighboring voxels. This filter size was empirically determined based on the registration results. Mean smoothed spatial maps for various timepoints are presented in FIGS. 10 and 12.

Sections from immunohistochemistry were also segmented for pathology and registered to the ARA using a similar computational pipeline, which was applied in two dimensions instead of three. For each brain section, the corresponding coronal ARA slice was first manually selected. The DAPI channel for each section was then registered to this atlas slice. Aggregates from the pSer129 channel were also detected using a machine learning model. For a given brain sample, the total aggregate count for each neuroanatomical region across all imaged histological sections was calculated. Since the histological sections only capture a sparse representation of the brain volume, each region's aggregate count was extrapolated by dividing by the total observed volume for that region, then multiplying by the total volume of that region in the ARA.

### Statistical Analysis

The smoothed maps from the image processing pipeline are used for two-sided T-tests at each voxel between samples at different timepoints. Due to the variability in the spreading patterns between adjacent time points, statistical tests were only run between time points with adequate spacing: 0.5 MPI vs 4 MPI, 4 MPI vs 8 MPI, and 8 MPI vs 18 MPI. Thus, the 2, 6, and 12 MPI time points were omitted. In order to account for the large number of voxels at a 25 m resolution, multiple comparison corrections were performed using the Benjamini-Hochberg method (Benjamini and Hochberg, 1995). The corrected p-values were thresholded at 0.05 for determining significance. Similar analysis was performed for counts when grouped into ARA anatomical regions.

### Computational Modeling

This Smoluchowski network model has been described extensively in previous studies (Fornari et al., 2020; Wattis, 2006), and is governed by the following set of differential equations.

$$\frac{d {c}_{1 , j}}{dt} = - α {\sum }_{k = 1}^{V} {L}_{jk} {c}_{1 , k} - {μ}_{1 , j} {c}_{1 , j} - 2 {c}_{1 , j}^{2} ( {κ}_{j} + ξ {\sum }_{k = 2}^{N} k {c}_{2 , k} ) - {c}_{1 , j} {\sum }_{k = 2}^{N} {c}_{k , j} \\ \frac{d {c}_{2 , j}}{dt} = - {2}^{- η} α {\sum }_{k = 1}^{V} {L}_{jk} {c}_{2 , k} - {2}^{λ} {μ}_{2 , j} {c}_{2 , j} + {c}_{1 , j}^{2} ( {κ}_{j} + ξ {\sum }_{k = 2}^{N} {kc}_{2 , k} ) - {c}_{1 , j} {c}_{2 , j} \\ \frac{d {c}_{i , j}}{dt} = - {i}^{- η} α {\sum }_{k = 1}^{V} {L}_{jk} {c}_{i , k} - {i}^{λ} {μ}_{ij} {c}_{ij} + {c}_{1 , j} ( {c}_{i - 1 , j} - ci , j ) \\ \frac{d {c}_{N , j}}{dt} = - {N}^{λ} {μ}_{N , j} {c}_{N , j} + {c}_{1 , j} {c}_{N - 1 , j}$$

c_{i,j} represents the total count of aggregates in the discretized size-bin indexed by l, in the brain region indexed by j. The L matrix represents the Laplacian matrix of the weighted directed graph connecting the various neuroanatomical regions of the brain, taken from the Allen Connectivity Atlas (Oh et al., 2014). As this system of differential equations has no closed form solution, numerical integration with the software package SciPy was used to solve for the state dynamics given the initial conditions. η was chosen as a hyperparameter that slows the spread of large aggregates as the inverse power of the size, while λ was chosen as a hyperparameter that accelerates the decay of aggregates proportionally to the power of their size. The initial values for α and μ, which control the rate of spreading between nodes and decay at a given node, respectively, were fit by sweeping through a 2D-grid (0.001 to 1000) and selecting the values that resulted in the lowest mean-squared error between the predicted and actual counts (α=0.037, μ=0.139). In the case of μ=0, k=0, ξ=0, and a one-dimensional size vector c, the above system of differential equations simplifies to the standard network diffusion model used in earlier studies (Henderson et al., 2019a; Pandya et al., 2019).

In order to quantify the model's sensitivity to specific neuroanatomical pathways in the brain, the Jacobian matrix was calculated by taking the partial derivative of the model's output with respect to the weight of the anatomical connection strength between two regions encoded into the model. An element of this matrix represents the relative importance of that anatomical connection in the spreading of aggregates to a specific region.

This model can be used to produce a ranking of the candidate seed locations for a given pathological state c at t=T MPI. To generate this ranking, each of the 424 neuroanatomical regions are used as separate seed locations at t=0 within the model and simulated forward in time to t=T MPI. Each of the 424 simulation results are then compared with the observed state c using a pairwise similarity metric. In this case, the similarity metric was the correlation coefficient between total regional aggregate counts across the observed and simulated states. The similarity metric values can then be used to sort the 424 seed locations as likely sites that lead to the observed pathological state c.

Similarly, the model can be used to predict the time since seeding t=T MPI for a given pathological state c. A property of this computational model is that the distribution of simulated aggregate sizes across the whole brain is invariant to which neuroanatomical region is used as the seed location at t=0. Therefore, the whole-brain distribution of aggregate sizes for state c can be compared with simulated distributions at various t using a pairwise similarity metric without taking the seed location into account. The mean squared error was calculated between the stimulated and observed distributions. When deciding among several candidate t values (0.5 MPI, 2 MPI, 4 MPI), the mean squared errors are inverted and normalized to sum to 1, providing a prediction probability for each t being the correct estimate of T for the given pathological state c.

In order to also encode regional genetic differences and evaluate their differential effect on model performance, we reconsider the α (spreading) and μ (decay) parameters as regionally dependent. The spreading from a specific region is made proportional to the gene density in that region. All genes are normalized to the same range so that we are only comparing the regional distribution of gene expression relative to that gene's total whole-brain expression. Since a is now considered a vector, the product of it with the Laplacian connectivity matrix L has the effect of modifying the regional connectivity encoded into the model. In order to preserve the model's previous fit to capturing the whole-brain longitudinal spread, we normalize each gene vector to have mean 1 and a standard deviation Σ, which is empirically set to preserve the correlation between predicted and observed whole-brain count. The normalization was chosen so that this product has the effect of maintaining the trace of the original matrix L. A derivation of this normalization preserving the trace of the Laplacian matrix is as follows: we assume the vector s is sampled from a multivariate normal distribution with mean-value 1 and standard deviation Σ.

$$s ∼ N ( 1 , \sum )$$

By using the definition of the matrix trace and representing s as a diagonal square matrix S, the trace of the product of S and the Laplacian matrix L results in the following, where l represents the diagonal of L.

$$Tr ( SL ) = Tr ( [ \begin{matrix}{s}_{1} & ⋯ & 0 \\ ⋮ & ⋱ & ⋮ \\ 0 & ⋯ & {s}_{V}\end{matrix} ] L ) = {\sum }_{i = 1}^{V} {s}_{i} {L}_{ii} = s \cdot diag ( L ) = s \cdot l$$

Thus, the trace is equivalent to the dot product of s and l, which has an expectation value equivalent to the sum of the entries of l, which recovers the definition of the trace of L.

$$s \cdot l ∼ N ( 1 \cdot l , l \sum l ) \\ E [ s \cdot l ] = Tr ( L )$$

After each gene is encoded into the model, its net effect on the regional correlation between the simulated and actual data is compared to the baseline correlation with no genes. This provides an ordered list of genes, ranked by the relevance of their spatial expression map in improving the regional predictions the model.

### REFERENCES

Aguzzi, A., and Calella, A. M. (2009). Prions: Protein Aggregation and Infectious Diseases. Physiol Rev 89, 1105-1152. 10.1152/physrev.00006.2009.
Aguzzi, A., and Rajendran, L. (2009). The transcellular spread of cytosolic amyloids, prions, and prionoids. Neuron 64, 783-790. 10.1016/j.neuron.2009.12.016.
Angot, E., Steiner, J. A., Hansen, C., Li, J. Y., and Brundin, P. (2010). Are synucleinopathies prion-like disorders? Lancet Neurol 9, 1128-1138. 10.1016/S1474-4422(10)70213-1.
Beach, T. G., Adler, C. H., Lue, L., Sue, L. I., Bachalakuri, J., Henry-Watson, J., Sasse, J., Boyer, S., Shirohi, S., Brooks, R., et al. (2009). Unified staging system for Lewy body disorders: correlation with nigrostriatal degeneration, cognitive impairment and motor dysfunction. Acta Neuropathol 117, 613-634. 10.1007/s00401-009-0538-8.
Benjamini, Y., and Hochberg, Y. (1995). Controlling the false discovery rate: a practical and powerful approach to multiple testing. Journal of the Royal statistical society: series B (Methodological) 57, 289-300.
Berg, S., Kutra, D., Kroeger, T., Straehle, C. N., Kausler, B. X., Haubold, C., Schiegg, M., Ales, J., Beier, T., Rudy, M., et al. (2019). ilastik: interactive machine learning for (bio) image analysis. Nature Methods 16, 1226-1232. 10.1038/s41592-019-0582-9.
Bieri, G., Brahic, M., Bousset, L., Couthouis, J., Kramer, N.J., Ma, R., Nakayama, L., Monbureau, M., Defensor, E., Schule, B., et al. (2019). LRRK2 modifies alpha-syn pathology and spread in mouse models and human neurons. Acta Neuropathol 137, 961-980. 10.1007/s00401-019-01995-0.
Braak, H., Del Tredici, K., Bratzke, H., Hamm-Clement, J., Sandmann-Keil, D., and Rub, U. (2002). Staging of the intracerebral inclusion body pathology associated with idiopathic Parkinson's disease (preclinical and clinical stages). J Neurol 249 Suppl 3, Ill/1-5. 10.1007/s00415-002-1301-4.
Braak, H., Del Tredici, K., Rub, U., de Vos, R. A., Jansen Steur, E. N., and Braak, E. (2003). Staging of brain pathology related to sporadic Parkinson's disease. Neurobiol Aging 24, 197-211. 10.1016/s0197-4580(02)00065-9.
Brahic, M., Bousset, L., Bieri, G., Melki, R., and Gitler, A. D. (2016). Axonal transport and secretion of fibrillar forms of alpha-synuclein, Abeta42 peptide and HTTExon 1. Acta Neuropathol 131, 539-548. 10.1007/s00401-016-1538-0.
Bryois, J., Skene, N. G., Hansen, T. F., Kogelman, L. J. A., Watson, H. J., Liu, Z. J., Brueggeman, L. O., Breen, E. O. M., Bulik, A., Arenas, E. N., et al. (2020). Genetic identification of cell types underlying brain complex traits yields insights into the etiology of Parkinson's disease. Nat Genet 52, 482-+. 10.1038/s41588-020-0610-9.
Challis, C., Hori, A., Sampson, T. R., Yoo, B. B., Challis, R. C., Hamilton, A. M., Mazmanian, S. K., Volpicelli-Daley, L. A., and Gradinaru, V. (2020). Gut-seeded alpha-synuclein fibrils promote gut dysfunction and brain pathology specifically in aged mice. Nat Neurosci 23, 327-336. 10.1038/s41593-020-0589-7.
Cushman, M., Johnson, B. S., King, O. D., Gitler, A. D., and Shorter, J. (2010). Prion-like disorders: blurring the divide between transmissibility and infectivity. J Cell Sci 123, 1191-1201. 10.1242/jcs.051672.
Desplats, P., Lee, H. J., Bae, E. J., Patrick, C., Rockenstein, E., Crews, L., Spencer, B., Masliah, E., and Lee, S. J. (2009). Inclusion formation and neuronal cell death through neuron-to-neuron transmission of alpha-synuclein. P Natl Acad Sci USA 106, 13010-13015. 10.1073/pnas.0903691106.
Dickson, D. W. (2012). Parkinson's Disease and Parkinsonism: Neuropathology. Csh Perspect Med 2. ARTN a009258 10.1101/cshperspect.a009258.
Fornari, S., Schafer, A., Kuhl, E., and Goriely, A. (2020). Spatially-extended nucleation-aggregation-fragmentation models for the dynamics of prion-like neurodegenerative protein-spreading in the brain and its connectome. J Theor Biol 486. ARTN 110102 10.1016/j.jtbi.2019.110102.
Gaspar, R., Meisl, G., Buell, A. K., Young, L., Kaminski, C. F., Knowles, T. P. J., Sparr, E., and Linse, S. (2017). Secondary nucleation of monomers on fibril surface dominates alpha-synuclein aggregation and provides autocatalytic amyloid amplification. Q Rev Biophys 50, e6. 10.1017/S0033583516000172.
Ghee, M., Melki, R., Michot, N., and Mallet, J. (2005). PA700, the regulatory complex of the 26S proteasome, interferes with alpha-synuclein assembly. FEBS J 272, 4023-4033. 10.1111/j.1742-4658.2005.04776.x.
Goedert, M., Clavaguera, F., and Tolnay, M. (2010). The propagation of prion-like protein inclusions in neurodegenerative diseases. Trends Neurosci 33, 317-325. 10.1016/j.tins.2010.04.003.
Goedert, M., Spillantini, M. G., Del Tredici, K., and Braak, H. (2013). 100 years of Lewy pathology. Nat Rev Neurol 9, 13-24. 10.1038/nrneurol.2012.242.
Goetz, C. G. (2011). The history of Parkinson's disease: early clinical descriptions and neurological therapies. Cold Spring Harb Perspect Med 1, a008862. 10.1101/cshperspect.a008862.
Gribaudo, S., Tixador, P., Bousset, L., Fenyi, A., Lino, P., Melki, R., Peyrin, J. M., and Perrier, A. L. (2019). Propagation of alpha-Synuclein Strains within Human Reconstructed Neuronal Network. Stem Cell Rep 12, 230-244. 10.1016/j.stemcr.2018.12.007.
Guo, J. L., and Lee, V. M. Y. (2014). Cell-to-cell transmission of pathogenic proteins in neurodegenerative diseases. Nat Med 20, 130-138. 10.1038/nm.3457.
Hansen, C., Angot, E., Bergstrom, A. L., Steiner, J. A., Pieri, L., Paul, G., Outeiro, T. F., Melki, R., Kallunki, P., Fog, K., et al. (2011). alpha-Synuclein propagates from mouse brain to grafted dopaminergic neurons and seeds aggregation in cultured human cells. J Clin Invest 121, 715-725. 10.1172/Jci43366.
Henderson, M. X., Cornblath, E. J., Darwich, A., Zhang, B., Brown, H., Gathagan, R. J., Sandler, R. M., Bassett, D. S., Trojanowski, J. Q., and Lee, V. M. Y. (2019a). Spread of alpha-synuclein pathology through the brain connectome is modulated by selective vulnerability and predicted by network analysis. Nat Neurosci 22, 1248-+. 10.1038/s41593-019-0457-5.
Henderson, M. X., Sedor, S., McGeary, I., Cornblath, E. J., Peng, C., Riddle, D. M., Li, H. L., Zhang, B., Brown, H. J., Olufemi, M. F., et al. (2020). Glucocerebrosidase Activity Modulates Neuronal Susceptibility to Pathological alpha-Synuclein Insult. Neuron 105, 822-836 e827. 10.1016/j.neuron.2019.12.004.
Henderson, M. X., Trojanowski, J. Q., and Lee, V. M. (2019b). alpha-Synuclein pathology in Parkinson's disease and related alpha-synucleinopathies. Neurosci Lett 709, 134316. 10.1016/j.neulet.2019.134316.
Henrich, M. T., Geibl, F. F., Lakshminarasimhan, H., Stegmann, A., Giasson, B. I., Mao, X., Dawson, V. L., Dawson, T. M., Oertel, W. H., and Surmeier, D. J. (2020). Determinants of seeding and spreading of alpha-synuclein pathology in the brain. Sci Adv 6. 10.1126/sciadv.abc2487.
Jucker, M., and Walker, L. C. (2013). Self-propagation of pathogenic protein aggregates in neurodegenerative diseases. Nature 501, 45-51. 10.1038/nature12481.
Kalia, L. V., and Lang, A. E. (2015). Parkinson's disease. Lancet 386, 896-912. 10.1016/S0140-6736(14)61393-3.
Kim, S., Kwon, S. H., Kam, T. I., Panicker, N., Karuppagounder, S. S., Lee, S., Lee, J. H., Kim, W. R., Kook, M., Foss, C. A., et al. (2019). Transneuronal Propagation of Pathologic alpha-Synuclein from the Gut to the Brain Models Parkinson's Disease. Neuron 103, 627-641 e627. 10.1016/j.neuron.2019.05.035.
Konno, M., Hasegawa, T., Baba, T., Miura, E., Sugeno, N., Kikuchi, A., Fiesel, F. C., Sasaki, T., Aoki, M., Itoyama, Y., and Takeda, A. (2012). Suppression of dynamin GTPase decreases alpha-synuclein uptake by neuronal and oligodendroglial cells: a potent therapeutic target for synucleinopathy. Mol Neurodegener 7, 38. 10.1186/1750-1326-7-38.
Kordower, J. H., Chu, Y., Hauser, R. A., Freeman, T. B., and Olanow, C. W. (2008). Lewy body-like pathology in long-term embryonic nigral transplants in Parkinson's disease. Nat Med 14, 504-506. 10.1038/nm1747.
Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007). Genome-wide atlas of gene expression in the adult mouse brain. Nature 445, 168-176. 10.1038/nature05453.
Li, J. Y., Englund, E., Holton, J. L., Soulet, D., Hagell, P., Lees, A. J., Lashley, T., Quinn, N. P., Rehncrona, S., Bjorklund, A., et al. (2008). Lewy bodies in grafted neurons in subjects with Parkinson's disease suggest host-to-graft disease propagation. Nat Med 14, 501-503. 10.1038/nm1746.
Luk, K. C., Kehm, V., Carroll, J., Zhang, B., O'Brien, P., Trojanowski, J. Q., and Lee, V. M. (2012a). Pathological alpha-synuclein transmission initiates Parkinson-like neurodegeneration in nontransgenic mice. Science 338, 949-953. 10.1126/science.1227157.
Luk, K. C., Kehm, V. M., Zhang, B., O'Brien, P., Trojanowski, J. Q., and Lee, V. M. (2012b). Intracerebral inoculation of pathological alpha-synuclein initiates a rapidly progressive neurodegenerative alpha-synucleinopathy in mice. J Exp Med 209, 975-986. 10.1084/jem.20112457.
Luna, E., Decker, S. C., Riddle, D. M., Caputo, A., Zhang, B., Cole, T., Caswell, C., Xie, S. X., Lee, V. M. Y., and Luk, K. C. (2018). Differential alpha-synuclein expression contributes to selective vulnerability of hippocampal neuron subpopulations to fibril-induced toxicity. Acta Neuropathol 135, 855-875. 10.1007/s00401-018-1829-8.
Oh, S. W., Harris, J. A., Ng, L., Winslow, B., Cain, N., Mihalas, S., Wang, Q., Lau, C., Kuan, L., Henry, A. M., et al. (2014). A mesoscale connectome of the mouse brain. Nature 508, 207-214. 10.1038/nature13186.
Oliveira, L. M. A., Gasser, T., Edwards, R., Zweckstetter, M., Melki, R., Stefanis, L., Lashuel, H. A., Sulzer, D., Vekrellis, K., Halliday, G. M., et al. (2021). Alpha-synuclein research: defining strategic moves in the battle against Parkinson's disease. NPJ Parkinsons Dis 7, 65. 10.1038/s41531-021-00203-9.
Pandya, S., Zeighami, Y., Freeze, B., Dadar, M., Collins, D. L., Dagher, A., and Raj, A. (2019). Predictive model of spread of Parkinson's pathology using network diffusion. Neuroimage 192, 178-194. 10.1016/j.neuroimage.2019.03.001.
Paumier, K. L., Luk, K. C., Manfredsson, F. P., Kanaan, N. M., Lipton, J. W., Collier, T. J., Steece-Collier, K., Kemp, C. J., Celano, S., Schulz, E., et al. (2015). Intrastriatal injection of pre-formed mouse alpha-synuclein fibrils into rats triggers alpha-synuclein pathology and bilateral nigrostriatal degeneration. Neurobiology of Disease 82, 185-199. 10.1016/j.nbd.2015.06.003.
Peelaerts, W., Bousset, L., Van der Perren, A., Moskalyuk, A., Pulizzi, R., Giugliano, M., Van den Haute, C., Melki, R., and Baekelandt, V. (2015). alpha-Synuclein strains cause distinct synucleinopathies after local and systemic administration. Nature 522, 340-344. 10.1038/nature14547.
Peng, C., Gathagan, R. J., Covell, D. J., Medellin, C., Stieber, A., Robinson, J. L., Zhang, B., Pitkin, R. M., Olufemi, M. F., Luk, K. C., et al. (2018). Cellular milieu imparts distinct pathological alpha-synuclein strains in alpha-synucleinopathies. Nature 557, 558-563. 10.1038/s41586-018-0104-4.
Prusiner, S. B. (1998). Prions. Proc Natl Acad Sci USA 95, 13363-13383. 10.1073/pnas.95.23.13363.
Prusiner, S. B., Woerman, A. L., Mordes, D. A., Watts, J. C., Rampersaud, R., Berry, D. B., Patel, S., Oehler, A., Lowe, J. K., Kravitz, S. N., et al. (2015). Evidence for alpha-synuclein prions causing multiple system atrophy in humans with parkinsonism. Proc Natl Acad Sci USA 112, E5308-5317. 10.1073/pnas.1514475112.
Recasens, A., Dehay, B., Bove, J., Carballo-Carbajal, I., Dovero, S., Perez-Villalba, A., Fernagut, P. O., Blesa, J., Parent, A., Perier, C., et al. (2014). Lewy body extracts from Parkinson disease brains trigger alpha-synuclein pathology and neurodegeneration in mice and monkeys. Ann Neurol 75, 351-362. 10.1002/ana.24066.
Renier, N., Adams, E. L., Kirst, C., Wu, Z., Azevedo, R., Kohl, J., Autry, A. E., Kadiri, L., Umadevi Venkataraju, K., Zhou, Y., et al. (2016). Mapping of Brain Activity by Automated Volume Analysis of Immediate Early Genes. Cell 165, 1789-1802. 10.1016/j.cell.2016.05.007.
Renier, N., Wu, Z., Simon, D. J., Yang, J., Ariel, P., and Tessier-Lavigne, M. (2014). iDISCO: a simple, rapid method to immunolabel large tissue samples for volume imaging. Cell 159, 896-910. 10.1016/j.cell.2014.10.010.
Sacino, A. N., Brooks, M., Thomas, M. A., McKinney, A. B., Lee, S., Regenhardt, R. W., McGarvey, N. H., Ayers, J. I., Notterpek, L., Borchelt, D. R., et al. (2014). Intramuscular injection of alpha-synuclein induces CNS alpha-synuclein pathology and a rapid-onset motor phenotype in transgenic mice. Proc Natl Acad Sci USA 111, 10732-10737. 10.1073/pnas.1321785111.
Shimozawa, A., Ono, M., Takahara, D., Tarutani, A., Imura, S., Masuda-Suzukake, M., Higuchi, M., Yanai, K., Hisanaga, S. I., and Hasegawa, M. (2017). Propagation of pathological alpha-synuclein in marmoset brain. Acta Neuropathol Commun 5, 12. 10.1186/s40478-017-0413-0.
Spillantini, M. G., Schmidt, M. L., Lee, V. M., Trojanowski, J. Q., Jakes, R., and Goedert, M. (1997). Alpha-synuclein in Lewy bodies. Nature 388, 839-840. 10.1038/42166.
Surmeier, D. J., Obeso, J. A., and Halliday, G. M. (2017). Parkinson's Disease Is Not Simply a Prion Disorder. J Neurosci 37, 9799-9807. 10.1523/JNEUROSCI.1787-16.2017.
Taguchi, K., Watanabe, Y., Tsujimura, A., Tatebe, H., Miyata, S., Tokuda, T., Mizuno, T., and Tanaka, M. (2014). Differential expression of alpha-synuclein in hippocampal neurons. PLoS One 9, e89327. 10.1371/journal.pone.0089327.
Tasic, B., Yao, Z., Graybuck, L. T., Smith, K. A., Nguyen, T. N., Bertagnolli, D., Goldy, J., Garren, E., Economo, M. N., Viswanathan, S., et al. (2018). Shared and distinct transcriptomic cell types across neocortical areas. Nature 563, 72-78. 10.1038/s41586-018-0654-5.
Volpicelli-Daley, L. A., Luk, K. C., Patel, T. P., Tanik, S. A., Riddle, D. M., Stieber, A., Meaney, D. F., Trojanowski, J. Q., and Lee, V. M. (2011). Exogenous alpha-synuclein fibrils induce Lewy body pathology leading to synaptic dysfunction and neuron death. Neuron 72, 57-71. 10.1016/j.neuron.2011.08.033.
Wattis, J. A. (2006). An introduction to mathematical models of coagulation-fragmentation processes: a discrete deterministic mean-field approach. Physica D: Nonlinear Phenomena 222, 1-20.

TABLE 1
KEY RESOURCES

| | | |
|---|---|---|
| REAGENT or RESOURCE | SOURCE | IDENTIFIER |

Antibodies

| | | |
|---|---|---|
| Rabbit Anti-Human alpha | Abcam | RRID: AB_869973 |
| Synuclein, phospho (Ser129) | | |
| Monoclonal Antibody | | |
| Donkey Anti-Rabbit IgG | ThermoFisher | RRID: AB_2536183 |
| (H + L) Polyclonal | | |
| Antibody, Alexa Fluor 647 | | |
| Purified anti-α-Synuclein | BioLegend | Cat# MMS-5091 |
| Phospho (Ser129) Antibody | | |
| Goat anti-Mouse IgG2a | ThermoFisher | RRID: AB_2535776 |
| Cross-Adsorbed Secondary | | |
| Antibody, Alexa Fluor 555 | | |

Deposited Data

| | | |
|---|---|---|
| Allen Mouse Brain Atlas | Allen Institute | RRID: SCR_002978 |
| Allen Mouse Brain | Allen Institute | RRID: SCR_008848 |
| Connectivity Atlas | | |

Experimental Models: Organisms/Strains

| | | |
|---|---|---|
| C57BL/6J mice | Jackson | Strain #: 000664; |
| | Laboratory | RRID: IMSR_JAX: |
| | | 000664 |

Other

| | | |
|---|---|---|
| Ultramicroscope II | LaVision | Stanford Neuroscience |
| | Biotec | Microscopy Service |

TABLE S1
Anatomical Regions, Related to FIG. 2. 212 anatomical
brain regions (per hemisphere) from the Allen Brain
Atlas were used for statistical analysis and modeling.

| Acronym | Name |
|---|---|
| AAA | Anterior amygdalar area |
| ACAd | Anterior cingulate area, dorsal part |
| ACAv | Anterior cingulate area, ventral part |
| ACB | Nucleus accumbens |
| AD | Anterodorsal nucleus |
| AHN | Anterior hypothalamic nucleus |
| AId | Agranular insular area, dorsal part |
| AIp | Agranular insular area, posterior part |
| AIv | Agranular insular area, ventral part |
| AMB | Nucleus ambiguus |
| AMd | Anteromedial nucleus, dorsal part |
| AMv | Anteromedial nucleus, ventral part |
| AN | Ansiform lobule |
| AOB | Accessory olfactory bulb |
| AON | Anterior olfactory nucleus |
| APN | Anterior pretectal nucleus |
| ARH | Arcuate hypothalamic nucleus |
| AUDd | Dorsal auditory area |
| AUDp | Primary auditory area |
| AUDv | Ventral auditory area |
| AV | Anteroventral nucleus of thalamus |
| BLA | Basolateral amygdalar nucleus |
| BMA | Basomedial amygdalar nucleus |
| BST | Bed nuclei of the stria terminalis |
| CA1 | Field CA1 |
| CA2 | Field CA2 |
| CA3 | Field CA3 |
| CEA | Central amygdalar nucleus |
| CENT | Central lobule |
| CL | Central lateral nucleus of the thalamus |
| CLA | Claustrum |
| CLI | Central linear nucleus raphe |
| CM | Central medial nucleus of the thalamus |
| COAa | Cortical amygdalar area, anterior part |
| COAp | Cortical amygdalar area, posterior part |
| CP | Caudoputamen |
| CS | Superior central nucleus raphe |
| CUL | Culmen |
| CUN | Cuneiform nucleus |
| DCO | Dorsal cochlear nucleus |
| DG | Dentate gyrus |
| DMH | Dorsomedial nucleus of the hypothalamus |
| DN | Dentate nucleus |
| DP | Dorsal peduncular area |
| DR | Dorsal nucleus raphe |
| ECT | Ectorhinal area |
| ENTl | Entorhinal area, lateral part |
| ENTm | Entorhinal area, medial part, dorsal zone |
| EPd | Endopiriform nucleus, dorsal part |
| EPv | Endopiriform nucleus, ventral part |
| FL | Flocculus |
| FN | Fastigial nucleus |
| FRP | Frontal pole, cerebral cortex |
| FS | Fundus of striatum |
| GPe | Globus pallidus, external segment |
| GPi | Globus pallidus, internal segment |
| GRN | Gigantocellular reticular nucleus |
| GU | Gustatory areas |
| IA | Intercalated amygdalar nucleus |
| ICc | Inferior colliculus, central nucleus |
| ICd | Inferior colliculus, dorsal nucleus |
| ICe | Inferior colliculus, external nucleus |
| ILA | Infralimbic area |
| IMD | Intermediodorsal nucleus of the thalamus |
| IO | Inferior olivary complex |
| IP | Interposed nucleus |
| IPN | Interpeduncular nucleus |
| IRN | Intermediate reticular nucleus |
| LA | Lateral amygdalar nucleus |
| LAV | Lateral vestibular nucleus |
| LD | Lateral dorsal nucleus of thalamus |
| LGd | Dorsal part of the lateral geniculate complex |
| LGv | Ventral part of the lateral geniculate complex |
| LH | Lateral habenula |
| LHA | Lateral hypothalamic area |
| LP | Lateral posterior nucleus of the thalamus |
| LPO | Lateral preoptic area |
| LRN | Lateral reticular nucleus |
| LSc | Lateral septal nucleus, caudal (caudodorsal) part |
| LSr | Lateral septal nucleus, rostral (rostroventral) part |
| LSv | Lateral septal nucleus, ventral part |
| MA | Magnocellular nucleus |
| MARN | Magnocellular reticular nucleus |
| MD | Mediodorsal nucleus of thalamus |
| MDRNd | Medullary reticular nucleus, dorsal part |
| MDRNv | Medullary reticular nucleus, ventral part |
| MEA | Medial amygdalar nucleus |
| MEPO | Median preoptic nucleus |
| MGd | Medial geniculate complex, dorsal part |
| MGm | Medial geniculate complex, medial part |
| MGv | Medial geniculate complex, ventral part |
| MH | Medial habenula |
| MM | Medial mammillary nucleus |
| MOB | Main olfactory bulb |
| MOp | Primary motor area |
| MOs | Secondary motor area |
| MPN | Medial preoptic nucleus |
| MPO | Medial preoptic area |
| MPT | Medial pretectal area |
| MRN | Midbrain reticular nucleus |
| MS | Medial septal nucleus |
| MV | Medial vestibular nucleus |
| NDB | Diagonal band nucleus |
| NI | Nucleus incertus |
| NLL | Nucleus of the lateral lemniscus |
| NLOT | Nucleus of the lateral olfactory tract |
| NOD | Nodulus (X) |
| NOT | Nucleus of the optic tract |
| NPC | Nucleus of the posterior commissure |
| NTS | Nucleus of the solitary tract |
| ORBI | Orbital area, lateral part |
| ORBm | Orbital area, medial part |
| ORBvl | Orbital area, ventrolateral part |
| OT | Olfactory tubercle |
| PA | Posterior amygdalar nucleus |
| PAA | Piriform-amygdalar area |
| PAG | Periaqueductal gray |
| PAR | Parasubiculum |
| PARN | Parvicellular reticular nucleus |
| PB | Parabrachial nucleus |
| PCG | Pontine central gray |
| PERI | Perirhinal area |
| PF | Parafascicular nucleus |
| PFL | Paraflocculus |
| PG | Pontine gray |
| PGRNd | Paragigantocellular reticular nucleus, dorsal part |
| PGRNl | Paragigantocellular reticular nucleus, lateral part |
| PH | Posterior hypothalamic nucleus |
| PIR | Piriform area |
| PL | Prelimbic area |
| PMd | Dorsal premammillary nucleus |
| PO | Posterior complex of the thalamus |
| POL | Posterior limiting nucleus of the thalamus |
| POST | Postsubiculum |
| PP | Peripeduncular nucleus |
| PPN | Pedunculopontine nucleus |
| PRE | Presubiculum |
| PRM | Paramedian lobule |
| PRNc | Pontine reticular nucleus, caudal part |
| PRNr | Pontine reticular nucleus |
| PRP | Nucleus prepositus |
| PSV | Principal sensory nucleus of the trigeminal |
| PT | Parataenial nucleus |
| PTLp | Posterior parietal association areas |
| PVH | Paraventricular hypothalamic nucleus |
| PVT | Paraventricular nucleus of the thalamus |
| PVp | Periventricular hypothalamic nucleus, posterior part |
| PVpo | Periventricular hypothalamic nucleus, preoptic part |
| PYR | Pyramus (VIII) |
| RCH | Retrochiasmatic area |
| RE | Nucleus of reuniens |
| RH | Rhomboid nucleus |
| RM | Nucleus raphe magnus |
| RN | Red nucleus |
| RR | Midbrain reticular nucleus, retrorubral area |
| RSPagl | Retrosplenial area, lateral agranular part |
| RSPd | Retrosplenial area, dorsal part |
| RSPv | Retrosplenial area, ventral part |
| RT | Reticular nucleus of the thalamus |
| SBPV | Subparaventricular zone |
| SCm | Superior colliculus, motor related |
| SCs | Superior colliculus, sensory related |
| SF | Septofimbrial nucleus |
| SI | Substantia innominata |
| SIM | Simple lobule |
| SMT | Submedial nucleus of the thalamus |
| SNc | Substantia nigra, compact part |
| SNr | Substantia nigra, reticular part |
| SOC | Superior olivary complex |
| SPA | Subparafascicular area |
| SPFm | Subparafascicular nucleus, magnocellular part |
| SPFp | Subparafascicular nucleus, parvicellular part |
| SPIV | Spinal vestibular nucleus |
| SPVC | Spinal nucleus of the trigeminal, caudal part |
| SPVI | Spinal nucleus of the trigeminal, interpolar part |
| SPVO | Spinal nucleus of the trigeminal, oral part |
| SSp-bfd | Primary somatosensory area, barrel field |
| SSp-ll | Primary somatosensory area, lower limb |
| SSp-m | Primary somatosensory area, mouth |
| SSp-n | Primary somatosensory area, nose |
| SSp-tr | Primary somatosensory area, trunk |
| SSp-ul | Primary somatosensory area, upper limb |
| SSs | Supplemental somatosensory area |
| STN | Subthalamic nucleus |
| SUB | Subiculum |
| SUM | Supramammillary nucleus |
| SUT | Supratrigeminal nucleus |
| SUV | Superior vestibular nucleus |
| TEa | Temporal association areas |
| TR | Postpiriform transition area |
| TRN | Tegmental reticular nucleus |
| TRS | Triangular nucleus of septum |
| TT | Taenia tecta |
| TU | Tuberal nucleus |
| V | Motor nucleus of trigeminal |
| VAL | Ventral anterior-lateral complex of the thalamus |
| VCO | Ventral cochlear nucleus |
| VII | Facial motor nucleus |
| VISC | Visceral area |
| VISal | Anterolateral visual area |
| VISam | Anteromedial visual area |
| VISl | Lateral visual area |
| VISp | Primary visual area |
| VISpl | Posterolateral visual area |
| VISpm | posteromedial visual area |
| VM | Ventral medial nucleus of the thalamus |
| VMH | Ventromedial hypothalamic nucleus |
| VPL | Ventral posterolateral nucleus of the thalamus |
| VPM | Ventral posteromedial nucleus of the thalamus |
| VPMpc | Ventral posteromedial nucleus of the thalamus, |
| | parvicellular part |
| VTA | Ventral tegmental area |
| XII | Hypoglossal nucleus |

### Example 2

### Neuromodulation Modifies α-Synuclein Spreading Dynamics In Vivo and is Predicted by Changes in Whole-Brain Function

Optogenetics is a powerful tool for selectively stimulating specific neuronal cell types with high spatial and temporal specificity (13). In Alzheimer's Disease, recent studies have demonstrated that optogenetic stimulations at gamma frequencies can attenuate amyloid pathology (14), a discovery which has since led to both auditory and visual non-invasive alternatives (14, 15). In the case of synucleinopathies such as Parkinson's Disease, optogenetics has been shown to rescue motor symptoms in α-synuclein-induced disease models (16, 17), or as a potential treatment following parkinsonian neurodegeneration (18-20). However, no study to date has demonstrated that stimulation can be used in vivo to affect the spreading and aggregation of whole brain α-synuclein pathology. Existing treatments like deep brain stimulation are effective at correcting circuit imbalances caused by neurodegeneration in Parkinson's Disease (21). An intervention that can target susceptible neurons with spatial, temporal, and cell-type specificity and affect the formation of pathology would be an important complement to treating Parkinson's Disease and other neurodegenerative disorders.

Here, we introduce a method for altering disease progression and modulating the aggregation of α-syn at the whole brain level using cell-type specific stimulation of neuronal circuitry with optogenetics. Using whole brain clearing alongside quantitative mapping of pathology (22, 23), we demonstrate that repeated optogenetic stimulation affects the spreading dynamics of alpha-synuclein aggregates in a region-specific manner. To understand the mechanism behind this intervention of pathological spread, we additionally explore colocalization with functional activity. As many neurodegenerative diseases are progressive in nature with distinct neuroanatomical profiles, it is important to be able to design and evaluate interventions that can modify the underlying pathological disease state with spatial and temporal precision.

### Results

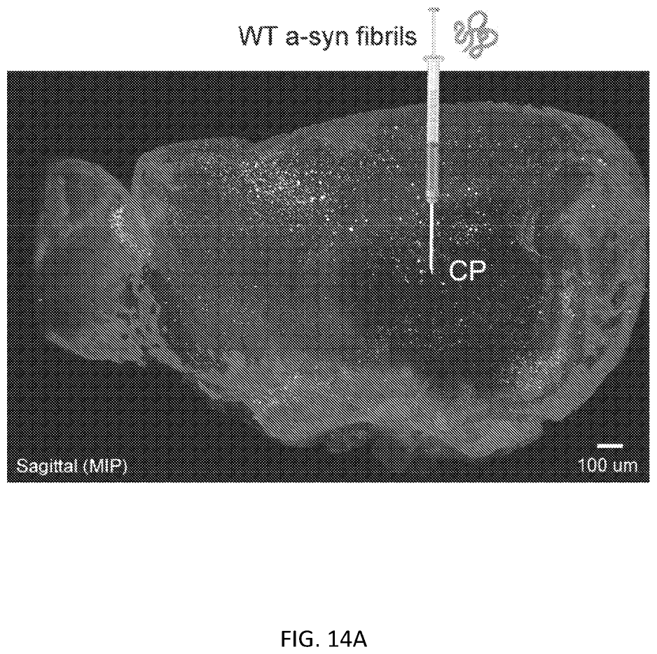

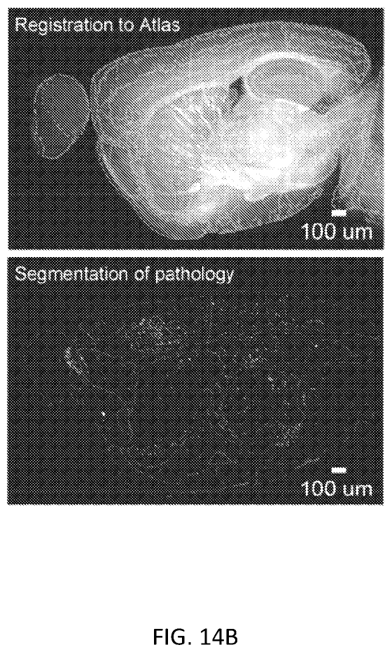

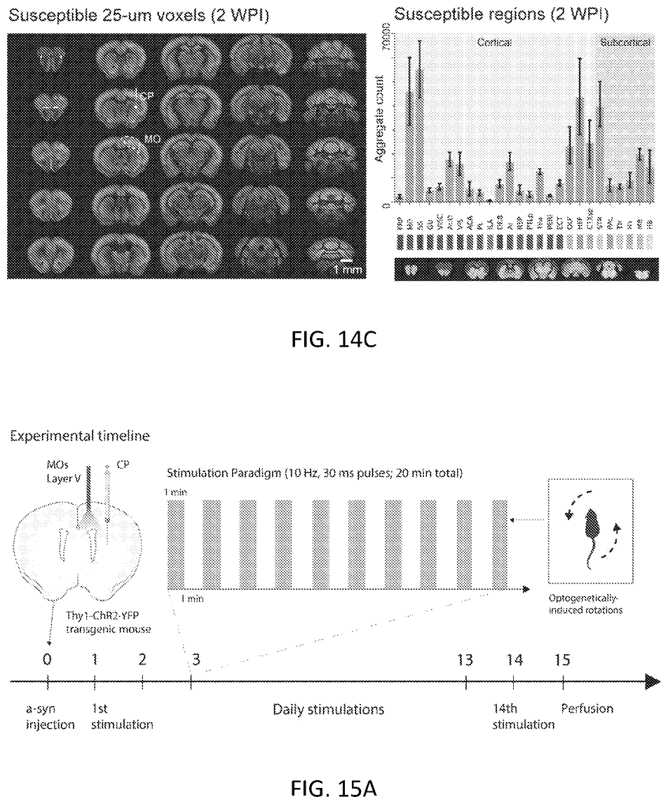

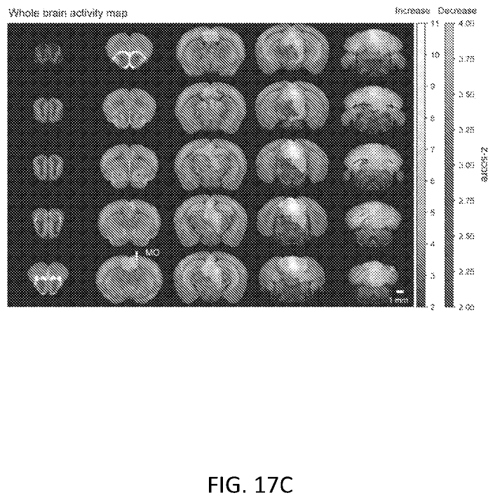

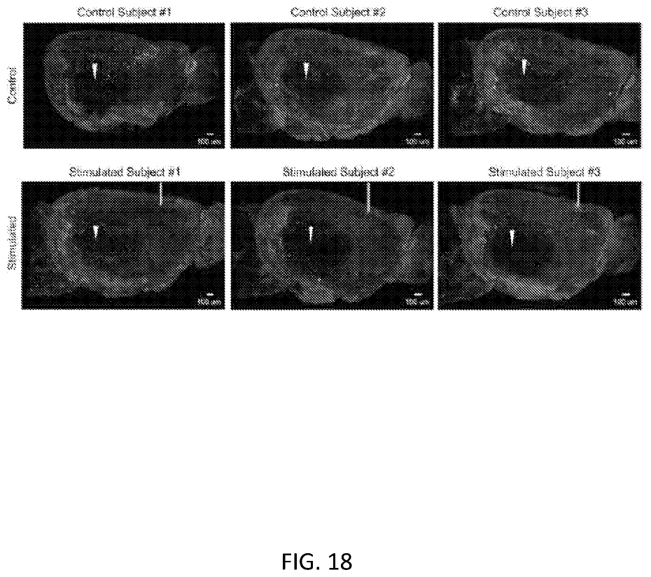

The direct injection of α-syn PFFs into the dorsal striatum results in widespread pathology throughout the brain, selective neurodegeneration, and Parkinsonian motor deficits (9). We measured whole brain pathology resulting from PFF injections using the tissue clearing and immunolabeling technique DISCO (23). DISCO allows for intact tissue to be labeled, cleared, and imaged in three dimensions without sectioning, allowing for the preservation and eventual quantification of three-dimensional α-syn aggregates throughout the whole brain. Following injection of PFFs into the striatum, extracted whole brains were immunolabeled, cleared, and imaged with light-sheet fluorescent microscopy (LSFM) (FIG. 14A, FIG. 18). We used previously-validated automated quantification pipelines, ClearMap and Ilastik (22, 24) for the segmentation of pathology and registration to the Allen Reference Atlas (ARA) (FIG. 14B) (25). For each subject, we automatically counted α-syn aggregates within both neuroanatomical regions and 25-mm voxels, uncovering the relative susceptibility of these regions of interest to pathology (FIG. 14C). This approach captured widespread pathology across the ipsilateral and contralateral hemispheres, consistent with previous reports of α-syn aggregation following PFF injections into the striatum (9, 10). Across a cohort of mice at two weeks post injection (WPI), we compared total aggregate counts across neuroanatomical regions from the ARA (FIG. 14C). The motor cortex (MO) harbored abundant α-syn pathology. A canonical pathway within the basal ganglia circuitry consists of projections from the cortex to striatum (26), and several recent studies have identified retrograde anatomical connectivity as a leading candidate for predicting downstream α-syn pathology (10, 27). Therefore, to stimulate motor cortex neurons that are involved in synaptic pathological spread, we chose to optogenetically target Layer 5 of the Secondary Motor Area in the ARA (28), which projects strongly to the striatum.

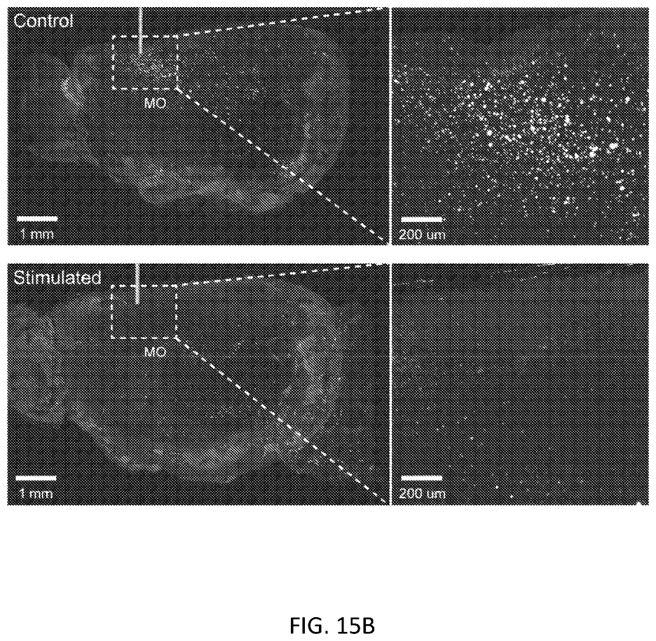

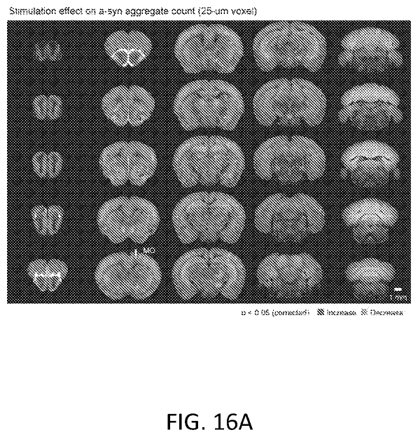

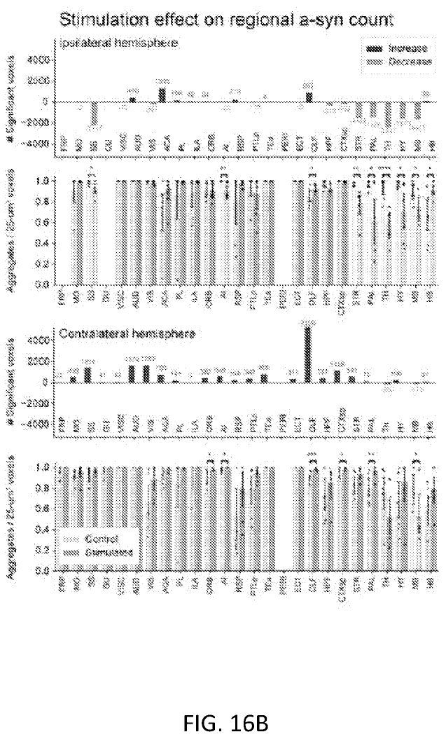

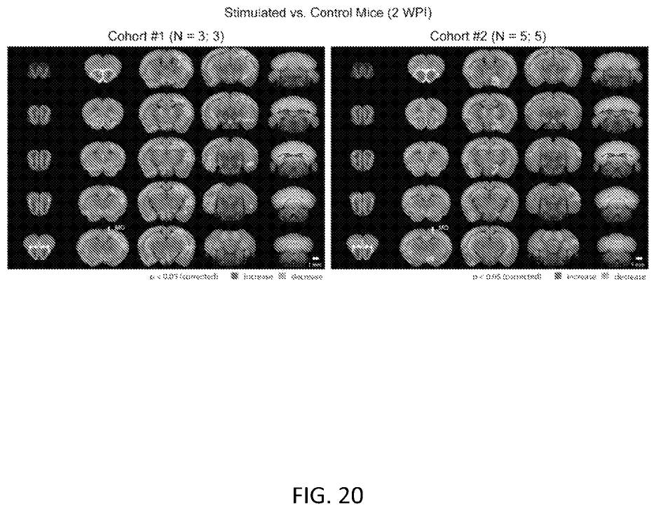

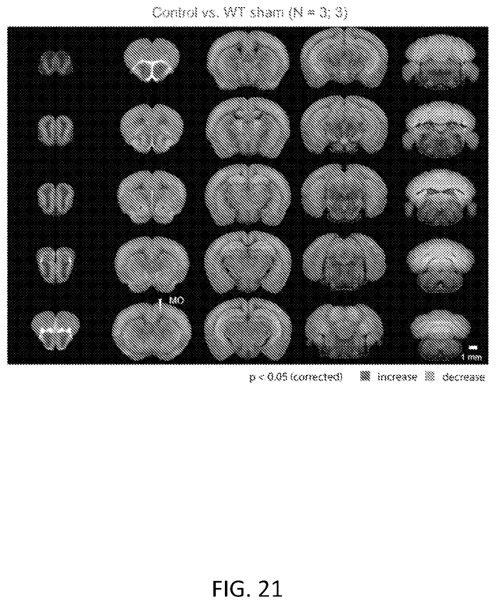

Following unilateral injection of α-syn PFFs into the striatum and two days of rest, we subjected a cohort of Thy1-ChR2 mice (N=8) in the treatment group to daily 20-minute stimulations for two weeks (FIG. 15A). We also injected a control group of Thy1-ChR2 mice (N=8) with α-syn PFFs and implanted them with an optical fiber, but they did not undergo the two-week stimulation. Following the two-week period, we used DISCO and LSFM to capture whole brain pathology for animals in both groups. Maximum intensity projections (MIP) of the raw data depict the effect of the stimulation on whole brain pathology (FIG. 15B), which show a striking decrease in pathology at the site of stimulation. To quantify and localize these effects, we performed voxel-level statistical tests across the two groups in the ARA space, which uncovered significant clusters of both increasing and decreasing aggregate count throughout the brain (FIG. 16A). Most notably, optogenetic stimulation decreased α-syn aggregation at the site of stimulation in the motor cortex and various subcortical regions but the entire contralateral cortex saw a markedly increased α-syn aggregate count. Statistical comparisons between neuroanatomical regions confirmed these findings (FIG. 16B). To validate whether a consistent outcome is obtained across different DISCO experiment batches, we performed these the experiments across two separate (N=3 (control), 3 (stimulation); N=5 (control), 5 (stimulation)) cohorts of animals. The resulting statistical maps from the two cohorts showed strikingly similar effects (FIG. 20). FIG. 16A depicts the intervention effect on these two combined cohorts (N=8 (control); N=8 (stimulation)). In order to account for potential effects of laser stimulation on pathology, such as heating or visual stimulation artifacts, we included an additional sham group of wild type mice (N=3) that underwent injection, implantation, and two-week mock laser stimulations. Whole brain pathology from this group showed no difference when compared with control wild type mice (N=3) (FIG. 21).

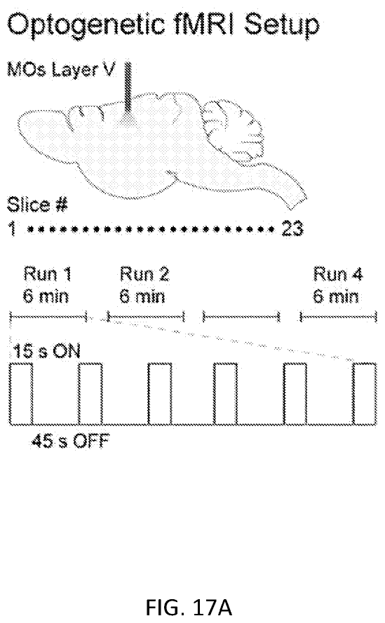

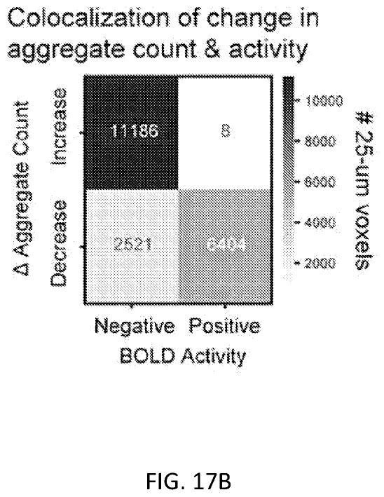

Optogenetic functional magnetic resonance imaging (ofMRI) is a technique for examining spatiotemporal changes in activity throughout the whole brain during cell-type-specific optogenetic stimulation (29, 30). We stimulated the same motor area with optogenetics during fMRI recording in order to measure changes in brain function caused by neurostimulation (FIG. 17A). Statistical analysis of these recordings resulted in activation maps indicating regions of significantly increased or decreased downstream neuronal activity (FIG. 17C). These activation maps, when colocalized with changes in pathology (FIG. 16A), showed remarkable similarity with opposite polarity (FIG. 17B). Positive activity, which represents active brain voxels significantly driven by the stimulation, showed high colocalization with a decrease in pathology. However, negative activity, which represents voxels significantly more active between stimulations, was highly localized to regions with increases in pathology.

In this study, we introduce a neurostimulation method for manipulating the spread of misfolded proteins throughout the whole brain. The ability to capture the whole brain pathological state using tissue clearing allows for a whole brain readout for determining changes in pathology caused by neurostimulation. Integration with ofMRI enabled colocalization of activation maps with changes in pathology, indicating that both polarity and localization of downstream activity during stimulation was highly indicative of resulting changes in pathology. More work is needed to uncover the full mechanism by which misfolded proteins such as α-syn spread throughout the connectome, and the biochemical pathways relating neuronal activity with modified spreading dynamics. However, the ability to readout and reliably manipulate the spread of pathological proteins like α-syn offers high clinical utility in the development of future therapeutics targeting neurodegenerative disease progression.

### Materials and Methods

Animals. Mouse husbandry and procedures were performed in accordance with institutional guidelines and approved by the Stanford Administrative Panel on Animal Care (APLAC). 10-12-week-old male Thy1-ChR2-YFP mice (The Jackson Laboratory, cat #007612) were used for stereotaxic injections. Mice were housed under specific pathogen-free conditions under a 12 h light-dark cycle, ad libitum diet and free access to water. Mice were excluded from the study if they experienced seizures following any of the daily stimulations. Hence, two total mice were excluded based on this criterion and were not used for tissue clearing and immunolabeling. A total of 25 mice were used in this study for tissue clearing: six mice (three in each control and stimulated group) for the initial cohort of two-week stimulations, ten mice (five in each control and stimulated group) for the follow-up cohort of two-week stimulations, and six mice (three in each control and sham group) for the comparisons between control and sham wild type mice with no opsin expression. An additional three mice were used for measuring brain activity using optogenetic fMRI.

PFF preparation. The expression and purification of mouse wild-type α-syn was performed as previously described (Ghee et al., 2005). The formation of α-Syn fibrils was induced by incubation in 50 mM Tris-HCl, pH 7.5, 150 mM KCl buffer at 37° C. under continuous shaking inside an Eppendorf Thermomixer at 600 rpm. α-Syn fibrils were centrifuged twice for 10 minutes at 15,000 g and then resuspended in PBS. All fibrils were fragmented prior to in vivo use by sonication for 20 min in 2-ml Eppendorf tubes in a Vial Tweeter powered by an ultrasonic processor UIS250v (250 W, 2.4 kHz; Hielscher Ultrasonic, Teltow, Germany). 5 ug of fibrils were used for each in vivo mouse experiment.

Fibril injections and Fiber implantation. Stereotaxic injections were performed on 10-12-week-old adult mice. Animals were placed in a stereotaxic frame and anesthetized with 2% isoflurane (2 L/min oxygen flow rate) delivered through an anesthesia nose cone. Ophthalmic eye ointment was applied to prevent desiccation of the cornea during surgery. The area around the incision was trimmed, cleaned, and disinfected. A small hole was drilled above the injection site. PFF solutions were injected unilaterally into the dorsal striatum using the following coordinates (from bregma): anterior=+0.4 mm, lateral=+/−1.85 mm from midline, depth=−2.7 mm (from dura). Mice were injected with sonicated PFFs (5 μg/mouse). 1 μl volume was injected at a rate of 100 nl/min using a 5 μl Hamilton syringe with a 32 G needle. To limit reflux along the injection track, the needle was maintained in situ for five minutes, before being slowly retrieved. The skin was closed with silk sutures.

After the fibril injection, a custom-designed fiber-optic implant was next mounted and secured on the skull using metabond (Parkell Inc.), with the optical fiber extending from the implant's base to the desired depth (˜0.2 mm above the stimulation site). The target stimulation site was in the Secondary Motor Area (MOs) Layer V, using the following coordinates: ML=−0.5 mm; AP=1.7 mm; DV=0.45 mm. Following surgery, mice were given buprenorphine (0.05 mg/kg, subcutaneously [s.c.]) twice daily for 2 days to minimize post-operative discomfort.

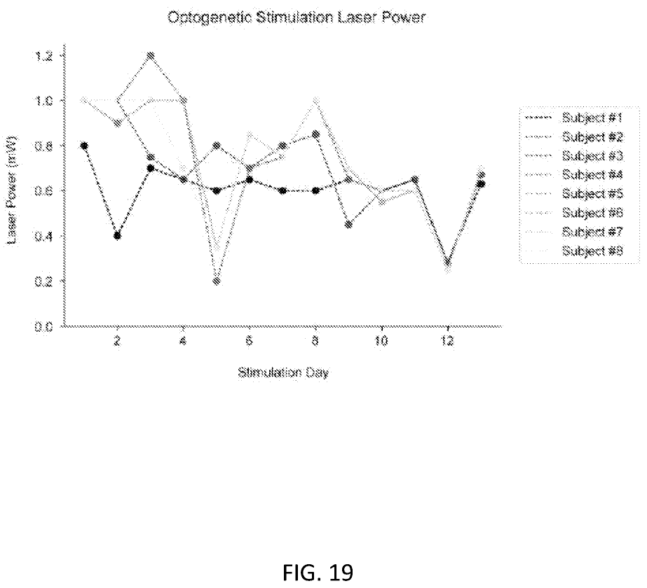

Optogenetic Stimulations. Mice in the treatment group were subjected to daily optogenetic stimulations for fourteen days, with one stimulation session per day. Each stimulation session consisted of ten alternating 1-minute stimulation and 1-minute rest blocks, totaling twenty minutes. The laser for optogenetic stimulation was delivered at 10 Hz with a 30% duty cycle, resulting in a 30 ms pulse width. Stimulation parameters were chosen based on physiological firing rates and the delivered laser power was minimized to only elicit a steady rotational bias in the mice. On each day of stimulation for a subject, the minimum laser power required to invoke robust rotational behavior was determined (FIG. 19).

Tissue Clearing. Each sample was fully immunolabeled and cleared using the previously described iDISCO protocol (Renier et al., 2014). An anti-phospho-synuclein (pSer129) Rabbit polyclonal antibody was used as the primary antibody at 1:1000 dilution, while a Donkey anti-Rabbit IgG (H+L) Alexa Fluor 647 nm antibody was used as a secondary antibody at 1:1000 dilution.

Microscopy. Each sample was imaged using an LaVision Biotec Ultramicroscope II within two days of finishing iDISCO clearing. Microscope settings of a full sheet width, numerical aperture of 0.103, mechanical step-size of 3.5 um, and light-sheet thickness of 7 um were used for all acquisitions. 4xx/488 nm excitation and emission filters were used for each autofluorescence acquisition, and 6xx/647 nm filters for detecting fluorescent α-synuclein pathology. The left and right hemispheres of each brain sample were imaged separately. The light-sheet, and thus each acquired slice, were acquired in the sagittal plane. Each acquisition had an in-plane resolution of 4.0625×4.0625 um, with a slice resolution of 3.5 um. Following light-sheet imaging, each acquired hemisphere was segmented for pathology and registered to a standardized atlas using ClearMap and Ilastik software (Renier et al., 2016, Berg et al., 2019).

Statistics. The smoothed maps of α-syn aggregate count were used for two-sided t-tests at each voxel between samples in two compared groups. In order to perform multiple comparisons corrections on the statistical maps, a non-parametric, threshold-free, cluster-based technique (Ridgway et al., 2012) was used to locate the clusters with significant change (p<0.05) while accounting for the correlation structure within these maps. This correction method considers both the distribution of cluster sizes and magnitudes of the test statistics within each cluster, which is necessary for capturing both diffuse and highly localized treatment effects. For statistical tests between neuroanatomical regions in the Allen Reference Atlas, multiple comparisons were corrected with the Benjamini-Hochberg method for setting the false discovery rate (Benjamini et al., 1995).

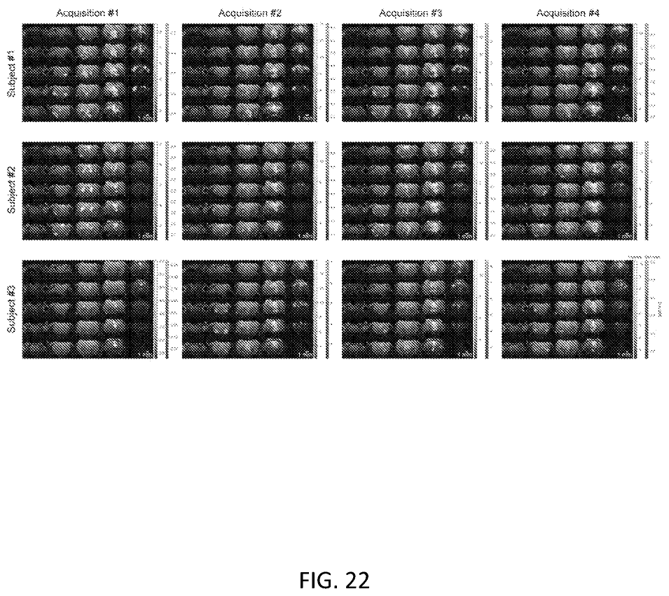

Optogenetic fMRI Experiments and Data Analysis. Optogenetic fMRI scanning was performed using a 7 Tesla Bruker Biospec small animal MRI system. Mice were scanned under very light anesthesia (0.3%-0.7% isoflurane mixed with O₂ and N₂O). Each ofMRI scan consisted of six 15 s pulse trains of optical stimulation delivered once per minute over 6 min. Most stimulation parameters during scanning were the same as those used for treatment (10 Hz, 30% duty cycle), except the laser power was increased to 1.5 mW to account for the effect of anesthesia. fMRI voxels significantly modulated by optogenetic stimulation were identified using generalized linear models in the FSL software package (Jenkinson et al., 2012). Individual activity maps presenting significant positive and negative activity during stimulation are presented in FIG. 22. Using the FSL registration package, registered scans were then averaged across the four acquisitions for each subject, then across all three subjects, resulting in the single activity map presented in FIG. 17.

### REFERENCES

1. M. Jucker, L. C. Walker, Self-propagation of pathogenic protein aggregates in neurodegenerative diseases. Nature 501, 45-51 (2013).
2. L. M. A. Oliveira et al., Alpha-synuclein research: defining strategic moves in the battle against Parkinson's disease. NPJ Parkinsons Dis 7, 65 (2021).
3. M. Goedert, M. G. Spillantini, K. Del Tredici, H. Braak, 100 years of Lewy pathology. Nat Rev Neurol 9, 13-24 (2013).
4. H. Braak et al., Staging of the intracerebral inclusion body pathology associated with idiopathic Parkinson's disease (preclinical and clinical stages). J Neurol 249 Suppl 3, Ill/1-5 (2002).
5. H. Braak et al., Staging of brain pathology related to sporadic Parkinson's disease. Neurobiol Aging 24, 197-211 (2003).
6. L. A. Volpicelli-Daley et al., Exogenous alpha-synuclein fibrils induce Lewy body pathology leading to synaptic dysfunction and neuron death. Neuron 72, 57-71 (2011).
7. V. M. Lee, J. Q. Trojanowski, Mechanisms of Parkinson's disease linked to pathological alpha-synuclein: new targets for drug discovery. Neuron 52, 33-38 (2006).
8. J. H. Kordower, Y. Chu, R. A. Hauser, T. B. Freeman, C. W. Olanow, Lewy body-like pathology in long-term embryonic nigral transplants in Parkinson's disease. Nat Med 14, 504-506 (2008).
9. K. C. Luk et al., Pathological alpha-synuclein transmission initiates Parkinson-like neurodegeneration in nontransgenic mice. Science 338, 949-953 (2012).
10. M. X. Henderson et al., Spread of alpha-synuclein pathology through the brain connectome is modulated by selective vulnerability and predicted by network analysis. Nat Neurosci 22, 1248-1257 (2019).
11. S. Pandya et al., Predictive model of spread of Parkinson's pathology using network diffusion. Neuroimage 192, 178-194 (2019).
12. M. T. Henrich et al., Determinants of seeding and spreading of alpha-synuclein pathology in the brain. Sci Adv 6, (2020).
13. O. Yizhar, L. E. Fenno, T. J. Davidson, M. Mogri, K. Deisseroth, Optogenetics in neural systems. Neuron 71, 9-34 (2011).
14. H. F. laccarino et al., Gamma frequency entrainment attenuates amyloid load and modifies microglia. Nature 540, 230-235 (2016).
15. A. J. Martorell et al., Multi-sensory Gamma Stimulation Ameliorates Alzheimer's-Associated Pathology and Improves Cognition. Cell 177, 256-271 e222 (2019).
16. C. Qi, S. Varga, S. J. Oh, C. J. Lee, D. Lee, Optogenetic Rescue of Locomotor Dysfunction and Dopaminergic Degeneration Caused by Alpha-Synuclein and EKO Genes. Exp Neurobiol 26, 97-103 (2017).
17. H. C. Moon et al., Effect of optogenetic modulation on entopeduncular input affects thalamic discharge and behavior in an AAV2-alpha-synuclein-induced hemiparkinson rat model. Neurosci Lett 662, 129-135 (2018).
18. A. V. Kravitz et al., Regulation of parkinsonian motor behaviours by optogenetic control of basal ganglia circuitry. Nature 466, 622-U627 (2010).
19. V. Gradinaru, M. Mogri, K. R. Thompson, J. M. Henderson, K. Deisseroth, Optical Deconstruction of Parkinsonian Neural Circuitry. Science 324, 354-359 (2009).
20. L. A. V. Magno et al., Optogenetic Stimulation of the M2 Cortex Reverts Motor Dysfunction in a Mouse Model of Parkinson's Disease. J Neurosci 39, 3234-3248 (2019).
21. M. S. Okun, Deep-Brain Stimulation for Parkinson's Disease. New England Journal of Medicine 367, 1529-1538 (2012).
22. N. Renier et al., Mapping of Brain Activity by Automated Volume Analysis of Immediate Early Genes. Cell 165, 1789-1802 (2016).
23. N. Renier et al., iDISCO: a simple, rapid method to immunolabel large tissue samples for volume imaging. Cell 159, 896-910 (2014).
24. S. Berg et al., ilastik: interactive machine learning for (bio) image analysis. Nature Methods 16, 1226-1232 (2019).
25. S. M. Sunkin et al., Allen Brain Atlas: an integrated spatio-temporal portal for exploring the central nervous system. Nucleic Acids Res 41, D996-D1008 (2013).
26. J. L. Lanciego, N. Luquin, J. A. Obeso, Functional Neuroanatomy of the Basal Ganglia. Cold Spring Harbor Perspectives in Medicine 2, (2012).
27. C. Mezias, N. Rey, P. Brundin, A. Raj, Neural connectivity predicts spreading of alpha-synuclein pathology in fibril-injected mouse models: Involvement of retrograde and anterograde axonal propagation. Neurobiology of Disease 134, (2020).
28. N. N. Foster et al., The mouse cortico-basal ganglia-thalamic network. Nature 598, 188-+(2021).
29. J. H. Lee et al., Global and local fMRI signals driven by neurons defined optogenetically by type and wiring. Nature 465, 788-792 (2010).
30. H. J. Lee et al., Activation of Direct and Indirect Pathway Medium Spiny Neurons Drives Distinct Brain-wide Responses. Neuron 91, 412-424 (2016).

### Example 3

Solving Brain Circuit Function and Dysfunction with Computational Modeling and Optogenetic fMRI

Can we systematically design treatments for brain disorders such as Parkinson's Disease? To enable this, we need a full algorithmic description, beyond simple correlations, of how specific cells or brain regions cooperatively contribute to the behavior or symptom in the context of whole-brain network. What does it take to generate such algorithms? Cell types cannot be ignored because even neurons that are in the same location of the brain can drive completely opposite functions and resulting behaviors (1, 2). Neurons often interact with large networks across the whole brain. A limited field of view within the brain is thus insufficient to understand these algorithms. Therefore, in order to obtain the data necessary to reconstruct these algorithms of behavioral control, we need an imaging system that can measure cell-type-specific, whole-brain function. Optogenetic functional magnetic resonance imaging (ofMRI) (3) has begun to achieve this goal. With ofMRI, we can select cell-type-specific modulation targets while monitoring the outcome of such modulation across the whole brain, in vivo with high spatiotemporal resolution. This has opened a new window into the study of brain function. We can see how modulating specific elements of the brain leads to specific behaviors of interest, while also directly observing the inner workings of the brain that led to such behaviors. Through computational modeling of ofMRI-signal measured across the whole-brain (4), cell-type-specific, large-scale brain function can be quantitatively described at the regional level. Once regional interaction maps are reconstructed, we envision that biophysical modeling can be combined to enable cell-type-specific, large-scale modeling of brain function at the single-cell-spiking level. In addition, while restoring brain function is the ultimate goal of neurological disease treatment, understanding how prominent pathology relates to brain function is also of critical importance. In this review, we will discuss approaches taken to date, and approaches that can be employed in the future towards these goals.

Bridging Scales with Optogenetic fMRI.

ofMRI is a technology that combines optogenetic stimulation with fMRI readout. Optogenetics (5, 6) enables cell-type-specific, millisecond-scale, activity modulation using light while high-field fMRI measures the resulting hemodynamic responses in live subjects across the whole brain. In the initial proof-of-concept study (3), motor cortex excitatory neurons triggered fMRI responses that could be measured throughout the brain with sub-second temporal resolution. To accelerate scientific discovery with ofMRI, several technological innovations were made. Real-time imaging with robustness to the live subject's motion that achieves data acquisition, reconstruction, motion correction (7), and analysis of 3D images with high accuracy in approximately 12 ms was developed. To resolve cortical layer and sub-nuclei specific responses, novel compressed sensing (8-10) and machine learning based fMRI technology was developed, which achieved significant reduction in voxel volume. MR-compatible optrodes and electrodes were also developed for simultaneous electrophysiological recordings to validate the neural basis of the ofMRI hemodynamic signal (11, 12). They can achieve simultaneous acquisition of electrophysiology recordings during fMRI sessions, and provide information with higher temporal resolution in regions of interest identified by ofMRI.

Utilizing these advanced ofMRI technologies, capabilities and precision of ofMRI has been extensively tested. It has been shown that location, polarity, and temporal shape of neural activity can be accurately inferred from the ofMRI signal (3, 13, 14), and that neural activity can be measured by ofMRI across multiple synapses (11, 15). Stimulation cell-type, location, frequency (14, 15), and intensity (11) was shown to dramatically change the location and shape of activities throughout the brain. It was also made clear that whole-brain neural dynamics as measured by ofMRI can accurately predict distinct behaviors (2, 14, 15).

Many studies have used ofMRI to improve our understanding of fundamental circuitries associated with behavior, memory, and cognition. For instance, ofMRI studies identified that frequency-dependent thalamic activities drive distinct whole-brain function in circuits associated with arousal, attention, and somatosensory function (16-18). ofMRI studies revealed distinct dorsal and ventral hippocampal control of brain-wide function (15, 19), circuits associated with cell-type-specific targeting of somatosensory cortex (20), and cerebellar cortex functional control over forebrain and midbrain (21). Another study revealed brain-wide dynamics that govern how the medial prefrontal cortex regulates reward-related behaviors through distant regions such as the striatum (22). A recent study has tried fMRI with cell-type-specific activation of astrocytes (23). These studies have shown that the observed hemodynamic activities are closely tied to neuronal activities using either simultaneous or follow-up electrophysiology. Furthermore, although most ofMRI studies have been conducted in rodents, it has also been applied to non-human primates, where both saccade latencies and whole-brain activity were found to be dependent on specific neuronal targets in the motor cortex (24).

The ability to probe and readout whole-brain activity with ofMRI has also advanced our understanding of dysfunctional circuitry associated with neurological disorders. For studying epilepsy, ofMRI provided a unique advantage of being able to optogenetically-induce seizures with precise origins on demand, while measuring the resulting whole-brain activities with simultaneous electrophysiology recordings. This enabled studies that can generate models to predict and classify seizures using its early activity markers (15, 25). Furthermore, longitudinal effects of seizures on global brain function could be measured to understand how the disease progresses and how seizures are generated and maintained (26). These advances aid our understanding of circuit mechanisms underlying seizures, help design intervention parameters, such as stimulation location and frequency to effectively inhibit seizures. Optogenetic fMRI can also elucidate mechanisms behind existing therapies, such as poststroke recovery. Activation as measured by ofMRI was highly predictive of the degree of recovery, and identified sensory circuits involved in this process (27).

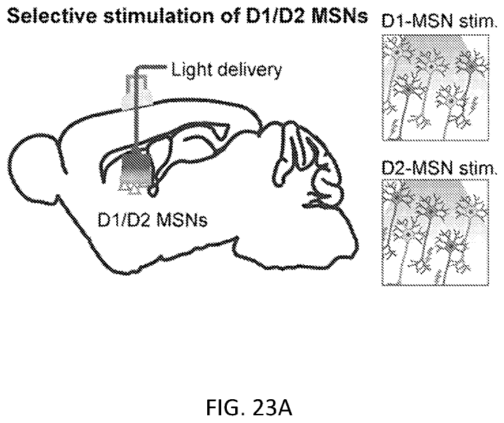

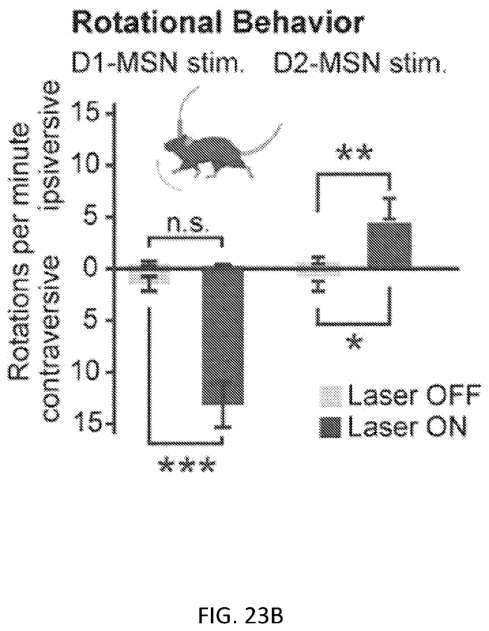

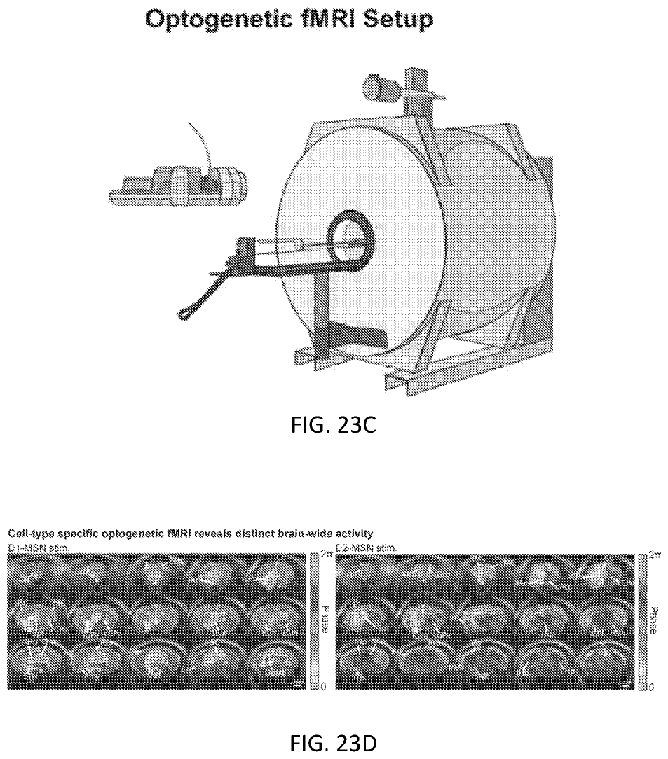

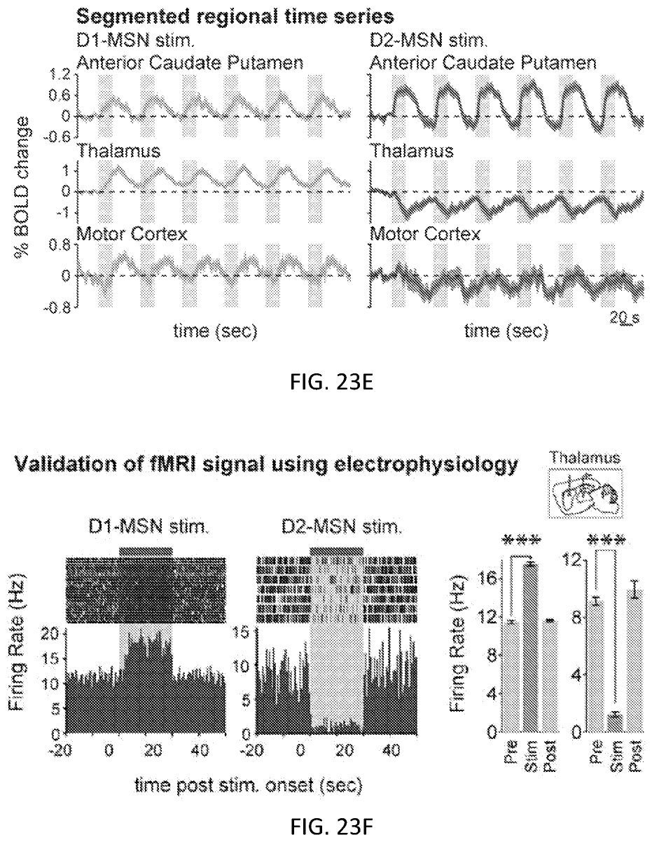

We can now start to reveal detailed circuit mechanisms that were challenging to understand before. As an example, we will review studies uncovering how D1- and D2-receptor expressing medium spiny neurons (MSNs) dynamically regulate global brain function and dysfunction (FIG. 23). The cortico-basal-ganglia-thalamus circuit is implicated in many important brain functions including motor control and reward mechanisms. Neurological disorders such as Parkinson's Disease, Huntington's Disease, addiction, and autism involve this network. The caudate putamen seats the D1- and D2-MSNs (FIG. 23A), which is the key node that separates the direct and indirect pathways (FIG. 23B). To assess the brain-wide dynamics driven by inhibitory D1- and D2-MSNs, Lee et al. performed whole-brain fMRI during repeated 20 s periods of optogenetic D1- or D2-MSN stimulations (2) (FIG. 23C). Active voxels were identified as those significantly synchronized to the repeated stimulations (FIG. 23D). The local signal at the site of stimulation was positive for both inhibitory D1- and D2-MSN stimulations (FIG. 23E), shedding light on a widely debated issue whether activity of inhibitory neurons evokes a positive or negative fMRI signal. At most regions of the ipsilateral cortico-basal-ganglia-thalamus network, the evoked response in a given region exhibited qualitatively different temporal profiles between D1- and D2-MSN stimulations (FIGS. 23D and 23E). Given the diversity of BOLD responses evoked by D1- and D2-MSN stimulations, we sought to verify whether the responses reflected underlying neuronal activity using single-unit recordings. For example, the fMRI time series in thalamus exhibited robust and reliable increases and decreases upon D1- and D2-MSN stimulations, respectively (FIGS. 23D and 23E). Indeed, single-units exhibited an increase in firing rate during D1-MSN stimulations and a decrease in firing rate during D2-MSN stimulations (FIG. 23F). These results demonstrate that ofMRI can detect cell-type-specific multi-synaptic activity changes across the whole brain with high spatiotemporal precision, and that electrophysiological recordings alongside ofMRI can support these findings.

As with any technology, ofMRI has caveats for future improvements as well as potential pitfalls that need to be avoided. Channelrhodopsin (ChR2) is known to evoke synchronized neuronal activity upon light stimulation. Therefore, before launching an ofMRI investigation, it is important to first investigate the behavioral impact of the optogenetic stimulations, as a mean to ensure that the behavior generated is of interest in either normal physiological or pathological context. For example, in our D1- and D2-MSN stimulation ofMRI experiments, increased contralateral and ipsilateral rotations were observed, respectively (2) (FIG. 23B). This shows that the two separate stimulations result in opposite behaviors known to be associated with movement disorders. The fact that ChR2 evokes synchronization upon light stimulation also makes it suitable for studying pathological oscillations in a number of disease models/contexts. Excessive beta-band oscillations in Parkinson's Disease have been extensively explored with ChR2 induced oscillations (28, 29). Some newer opsins, such as stabilized step function opsin (SSFO) (30), modulate target neurons by increasing the excitability to amplify existing spontaneous activity, which expands the application range of ofMRI to more physiological conditions. There are many aspects of ofMRI that can be further improved. For example, ofMRI will benefit greatly from higher spatio-temporal resolution, real-time feedback-based stimulation control, and imaging during behaviors (7, 31).

It is also important to note other technologies that have been developed for the investigation of brain circuitries, including high-speed volumetric calcium imaging (32, 33), probes for high-density electrophysiology recordings (34, 35), and widefield calcium and voltage imaging (36). Compared to ofMRI, these new advances offer higher spatiotemporal resolution, although limited by recording depths and field of view. For example, widefield “whole-cortex” calcium imaging enables up to 30 Hz simultaneous recording of cortical regions, but its depth coverage is limited to superficial layers of cortex. Combining fMRI and widefield calcium imaging has been shown to mitigate the limitations of both methods (36). Therefore, future ofMRI studies could be integrated and with other technologies for complementary strengths.

### Cell-Type-Specific Modeling of Large-Scale Brain Function.

Large-scale neural network models (37-41) utilizing experimental data from PET (42), fMRI (43), EEG/MEG (44) have made significant contributions to understanding brain functions. However, although these modeling efforts are based on data from carefully designed experiments that attempt to isolate specific brain function, multiple networks and pathways mediated by different cell types are simultaneously involved in orchestrating a brain function. Therefore, without the capability to untangle contributions from different cell types across the whole brain, models have been limited in their capabilities. The development of ofMRI technology opens a new opportunity in terms of whole-brain computational modeling because it uniquely measures cell-type-specific whole-brain dynamics.

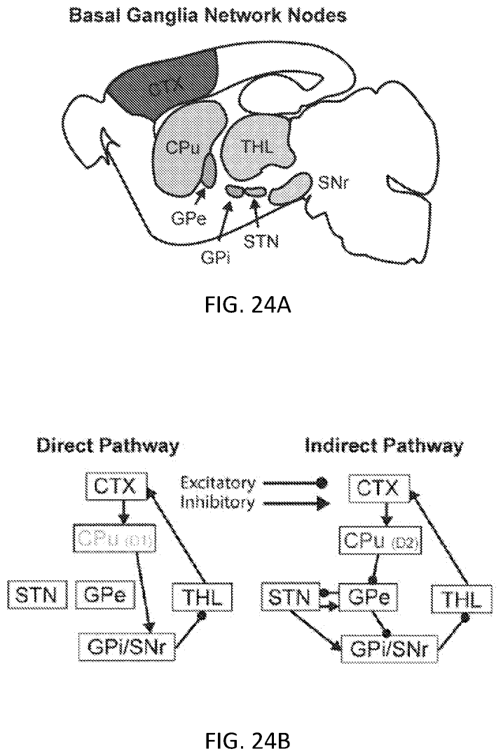

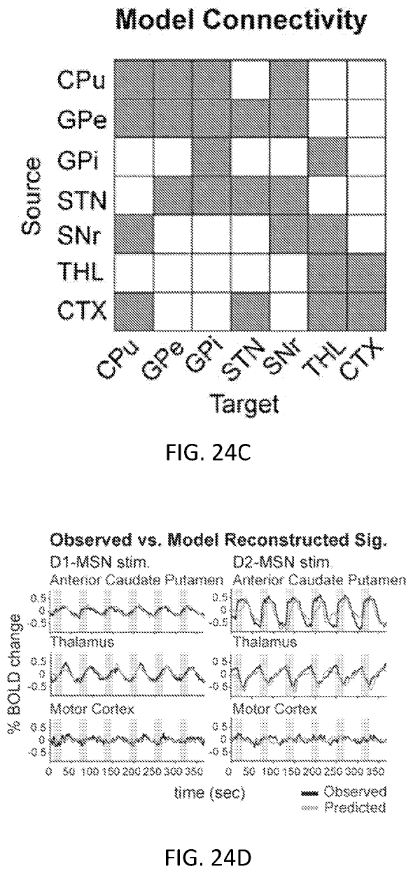

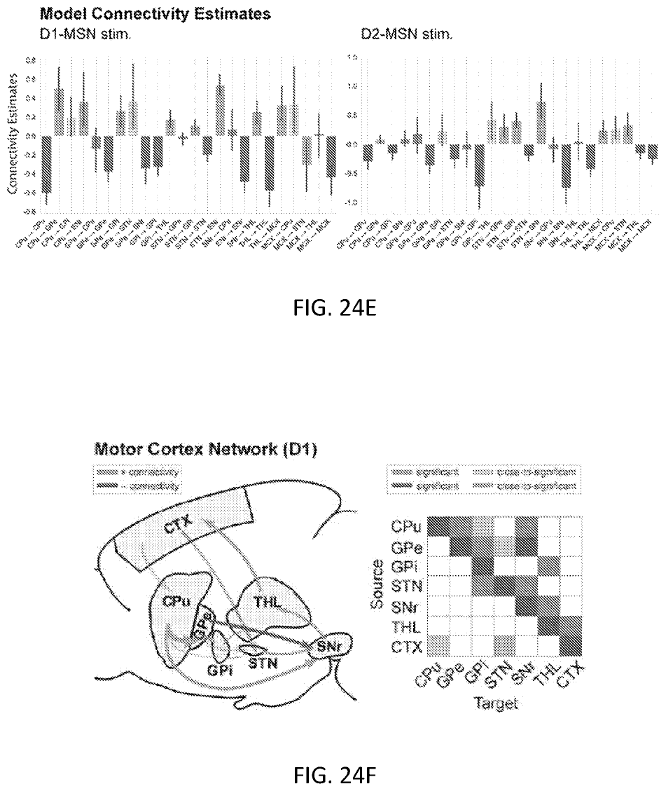

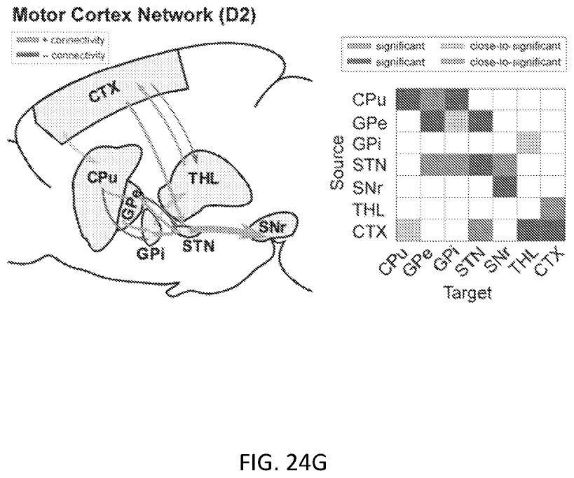

For example, the cortico-basal-ganglia-thalamus network features a large number of network nodes (FIG. 24A) and distinct cell types that have distinct long-range projections. According to the canonical functional model of basal ganglia (45, 46), movement initiation and suppression are mediated by the direct and indirect pathways, respectively (FIG. 24B). The direct pathway promotes movements through D1-MSN excitation in the striatum followed by excitation in the thalamus and consequently the motor cortex. On the other hand, the indirect pathway is assumed to transfer the activation of D2-MSN in the striatum via complex interactions within the basal ganglia, resulting in inhibition of the thalamus and consequently the motor cortex. However, despite the wide usage and success of the canonical model in explaining experimental results, recent experimental evidence argues against the model and finds the interactions between the two pathways still puzzling. For example, the direct and indirect pathways operate simultaneously during movement onsets (47).

As demonstrated earlier with ofMRI, we can decompose the operation of the cortico-basal-ganglia-thalamus network by optogenetically activating D1- and D2-MSNs selectively and directly observing the corresponding brain-wide dynamics (2) (FIG. 23). However, the degree of connectivity in the cortico-basal-ganglia-thalamus network is too high (FIGS. 24B and 24C) to identify regional causal influences based on the time series alone (FIG. 24D). Through computational modeling such as dynamic causal modeling (DCM), with the precision afforded by the ofMRI data, cell-type-specific causal linkages among regions of interest (ROIs) in the cortico-basal-ganglia-thalamus network could be identified.

DCM is a modeling scheme that estimates the causal coupling (effective connectivity) in a multi-region network based on neuroimaging data (fMRI, MEG/EEG) (48-50). The estimations are fitted to empirical results using Bayesian techniques. One major strength of DCM is that the estimated regional connectivities are directional, which is especially valuable for networks with a lot of reciprocal connections and feedbacks like the cortico-basal-ganglia-thalamus network. ofMRI can also be combined with other modeling schemes. For example, Salvan et al. (51) optogenetically modulated the entorhinal cortex and combined hidden Markov modeling with ofMRI data to study how entorhinal cortex drives frequency-dependent brain-wide dynamic states. In another study, multivariate dynamical systems (MDS) causal modeling was used with ofMRI to estimate causal brain interactions (52). Like DCM, MDS models both intrinsic and experimentally induced causal couplings in a large-scale brain network.

In our previous study (4), spectral DCM (38), a variation of DCM that enables large-scale networks to be modeled with computational efficiency, was used to investigate the interactions within the cortico-basal-ganglia-thalamus network with ofMRI data from D1- and D2-MSN optogenetic stimulations. One recent study reported consistent results using DCM with D1- and D2-MSN stimulation ofMRI data (53). As illustrated in FIG. 24, combining DCM or equivalent modeling schemes with ofMRI data can accurately reveal brain-wide regional interactions. The time series is also accurately reproduced by DCM, closely matching experimental ofMRI time series (FIG. 24D). FIGS. 24E-24G show the DCM estimations of between-region effective connectivity (4) utilizing ofMRI data. DCM results verified the direct pathway activation during D1-MSN stimulation and indirect pathway activation during D2-MSN stimulation. The defining connections of direct pathway model are statistically significant (CPu to SNr, GPi to thalamus, SNr to thalamus) or close-to-significant (CPu to GPi) with D1-MSN stimulations. In the D2-MSN stimulation network, significant connections included those of indirect pathway (CPu to GPe, GPe to STN, STN to GPi/SNr, and GPi to thalamus). The existence of cortical feedbacks can also be observed (FIGS. 24F and 24G). On the other hand, the effective connectivity estimates by DCM also suggest several positive projections that are anatomically inhibitory and cannot be explained by the canonical direct/indirect pathway model, such as the projections from GPi to thalamus during D1-MSN stimulations, which matches several experimental reports of GPi-thalamus paradoxical coactivation (2, 54-56). Understanding the mechanism underlying such paradoxical connections requires further microscopic investigations into the specific synaptic interactions with techniques like single-cell-spiking level modeling and single-unit recordings, as we will discuss next.

### Cell-Type-Specific, Single-Cell-Spiking Level Modeling of Brain-Wide Function.

One key strength of computational modeling is that it can bridge between spatial scales, from whole-brain dynamics to single-cell activity, and explain data from different experimental modalities, from fMRI to extracellular recordings (57, 58). DCM and other macroscale and mesoscale brain models commonly use neural mass models or mean-field models as the basic unit which describes the collective neural activity in a brain region or a cortical column (42-44). On the other hand, single-cell-spiking models computationally depict the microscopic biophysical features of how single-cell level spiking controls and modulates brain functions/dysfunctions (59-61).

Despite having the ability to capture and model the macroscale and mesoscale interactions between separate regional populations of neurons in the brain with ofMRI and DCM, there is certainly an added advantage in modeling the interactions between individual neurons of varying cell-type. Neurological disorders may differentially impact specific cell populations within one brain region. Optogenetically stimulating one type of cell population, while inhibiting another type in external globus pallidus (GPe) prolonged the therapeutic effects on a mouse model of Parkinson's Disease (52). It is widely assumed and supported by optogenetics studies that Parkinson's Disease causes hyperactivity of D2-MSNs and hypoactivity of D1-MSNs, thus impairing the balance between the direct and indirect pathways (46, 47, 62). DYT1 dystonia, a genetic early onset dystonia, is related to cholinergic interneuron dysfunction and altered D2 receptor-function in striatum (63, 64). Firing pattern alterations of one cell type may also contribute to large-scale changes. Optogenetic stimulation in striatal cholinergic interneurons, a subpopulation constituting less than 2% of the striatum, could generate broad-band oscillations in the motor network (29). With cell-type-specific, single-cell-spiking level modeling, it is easier to address the heterogeneity and rich microscopic interactions within one region with biophysical details. ofMRI-based DCM or other regional brain dynamics model can serve as a bridge between whole-brain dynamics and single-cell-spiking level activity, enabling construction of large-scale, cell-type-specific biophysical models that can test neuronal-level hypotheses.

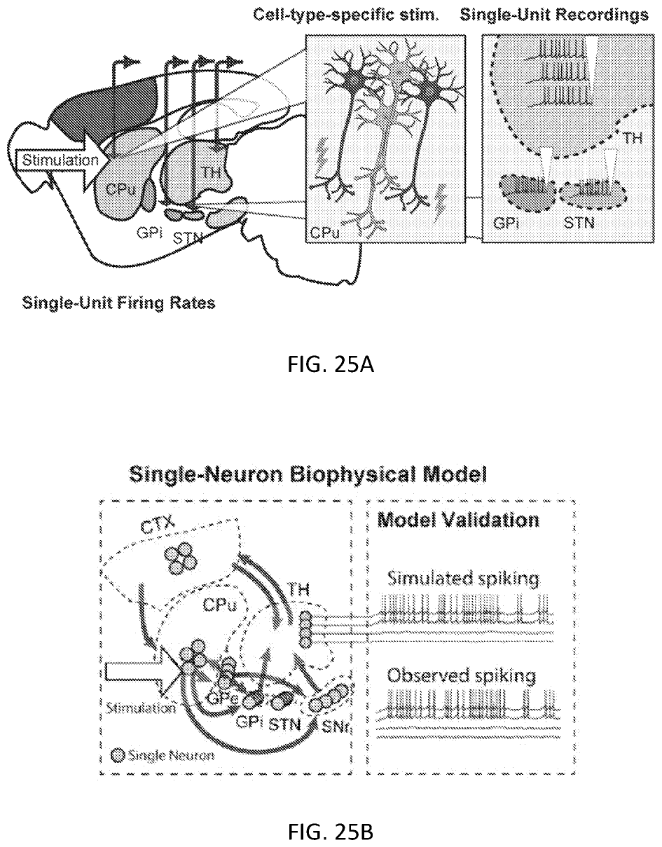

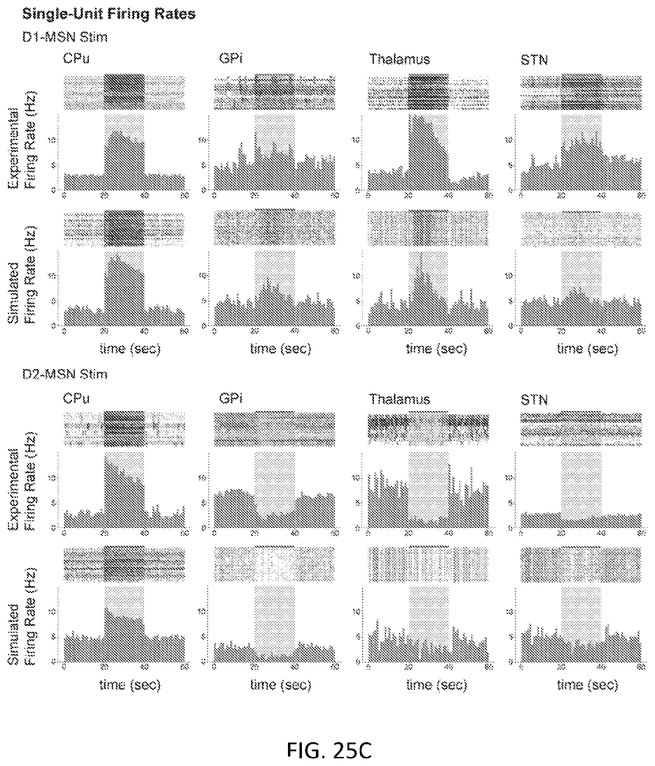

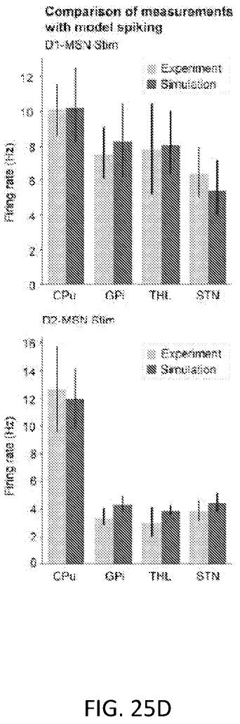

Cell-type-specific, single-cell-spiking level models that can accurately predict circuit function and dysfunction can be very powerful tools for designing or optimizing therapy. Thus far, many large-scale models without cell type specificity have been constructed to test various hypotheses underlying deep-brain stimulation (DBS), an existing therapy for Parkinson's Disease (65-67). However, because each cell type in the basal ganglia has distinct synaptic and physiological properties, accurate models would need cell-type-specific parameters that can fit cell-type-specifically acquired experimental data. We envision that ofMRI, DCM, and electrophysiological recordings can be combined to build large-scale biophysical models that can model brain-wide activity at the single-cell-spiking level. FIG. 25A demonstrates this vision. With ofMRI and electrophysiology experiments providing multi-scale data, and DCM providing brain-wide interaction information, single-cell level biophysical models, with accurate cell-type-specific, large-scale context, can be built and validated (FIG. 25B). As illustrated in FIGS. 25C and 25D, a single-cell-spiking level model can be designed to reproduce experimental neuronal activity and group dynamics with high precision. With such models, we can imagine simulating brain-wide spiking activity from “virtual neuromodulations” without needing to do in vivo experiments. We would then be able to use these results to better optimize therapeutic targets and parameters where simulated spiking matches our desired response. For example, a recent study (68) employed optogenetics and single-cell level computational modeling within three different cell types of the motor cortex to show that DBS on cortical somatostatin interneurons can rescue Parkinsonian symptoms. We envision that in the future, ofMRI-enabled brain-wide models with single-cell-spiking level precision will ultimately enable systematic design of neuromodulation therapy with predictable outcome.

### Modeling Whole-Brain Pathology Dynamics and its Relationship to Brain Function.

The brain circuitry is relevant to neurological disorders beyond its utility in modeling local and global brain function. It is also important for understanding the underlying pathology of many disorders. In the case of Parkinson's Disease and other synucleinopathies, the seeding and gradual accumulation of pathological alpha-synuclein (α-syn) causes dopaminergic neurodegeneration, which ultimately leads to striatal imbalance and the cardinal Parkinsonian signs (69). Although the specific biochemical trafficking mechanisms of these proteins remain unknown, several recent studies have shown that whole-brain spreading patterns are highly dependent on the inoculation site (70), and that anatomical connectivity is highly predictive of susceptible regions after induced seeding of α-syn pathology (71).

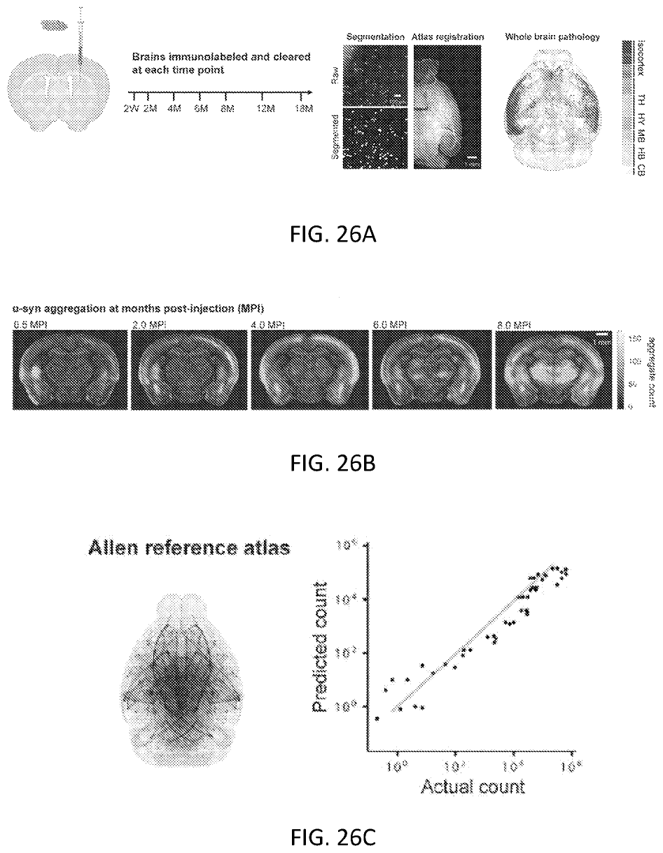

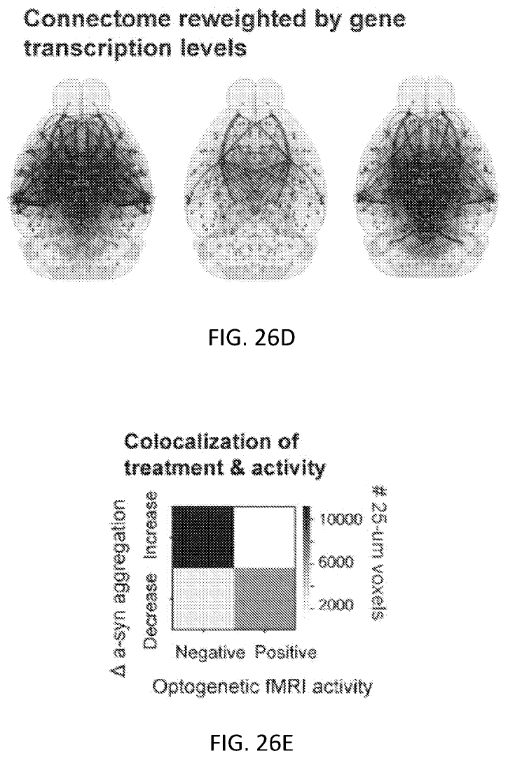

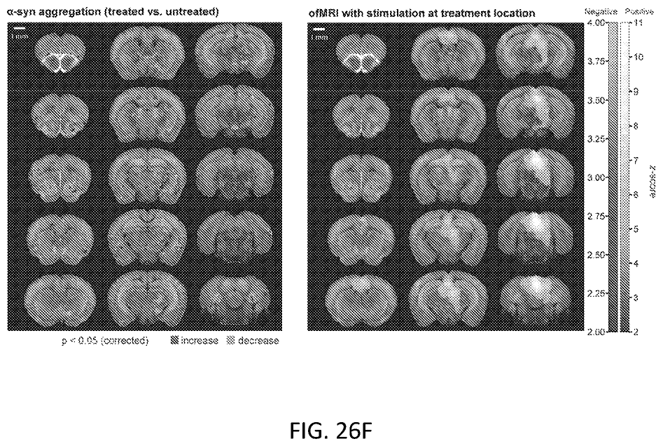

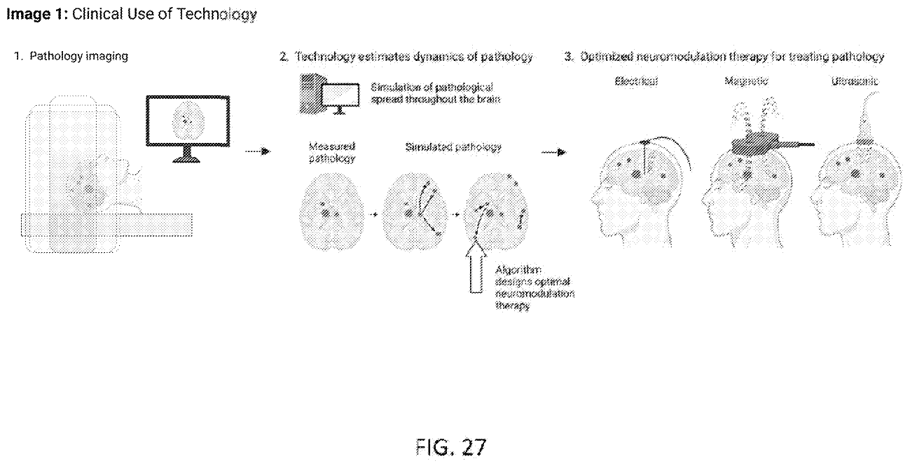

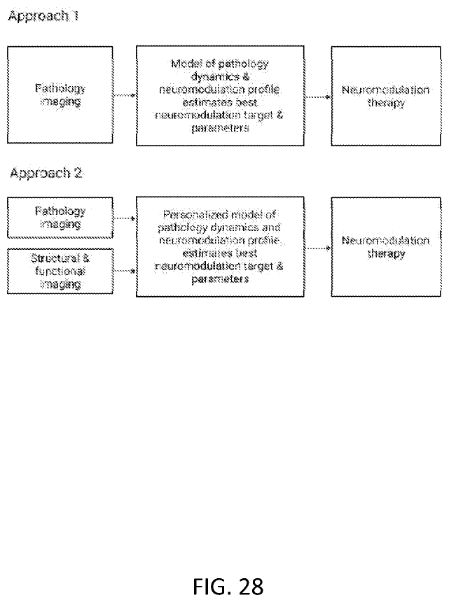

To understand the linkage between anatomical connections, pathology, and function, it is important to reliably measure the whole-brain pathological state. The last decade has seen several prominent advancements in whole-brain tissue clearing technology. These range from hydrogel-based techniques such as CLARITY (72) that enable multiplexed brain imaging but potentially require long incubation times, to solvent-based techniques like 3DISCO (73) that are generally faster but may bleach fluorescent signal. Combining these tissue clearing methods with whole-brain immunolabeling and light-sheet microscopy allows for high-resolution examination of whole-brain pathology using biochemical reporters (74, 75). As these three-dimensional whole-brain histological datasets now provide micron resolution and are exceeding terabytes in size, it is necessary to have automated registration and segmentation techniques (76) to capture the rich information provided by the data. FIG. 26A depicts how a Parkinsonian disease model where the injection of α-synuclein PFFs are used to trigger whole-brain pathology can be systematically analyzed using computational pipeline. Brains can be immunolabeled and cleared at each time point using the DISCO method (74), with the imaged aggregates automatically segmented and registered to a standardized atlas. As depicted in FIG. 26B, longitudinal analysis of pathology can demonstrate a highly dynamic, brain-wide process of spreading after seeding of α-syn, consistent with numerous histological studies (69, 71). Registration to a standardized atlas can provide an enormous advantage enabling systematic statistical comparisons and analysis of disease pathology alongside connectomic (77), genetic (78), and vascular (79) databases, which all reside in the same reference coordinate space. Most notably, the Allen Connectivity Atlas (77) has been crucial in the development of models describing the spread of pathology through the connectome (71). For instance, network diffusion models represent the whole-brain circuitry as a directed graph with neuroanatomical regions as nodes and axonal pathways as edges between these nodes (FIG. 26C). They have found wide applicability in predicting the spread of α-syn pathology in both rodent and human imaging studies (71, 80), as well as in human dementia (81) and supranuclear palsy neuroimaging studies (82). In FIG. 26C, we present an example of how a whole-brain model can accurately reconstruct the regional variability in pathology. Compared to previous models that depended on serial histological sectioning (71) or in vivo human imaging data (80), this type of modeling can provide a more comprehensive, higher resolution description of pathology dynamics that includes all brain regions, which is important for quantitative model descriptions. Given that transgenic mice such as those with modified LRRK2 (71, 83) or SNCA (69) expression demonstrate significantly altered spreading patterns, models can additionally incorporate the brain's inherent genetic or cell-type-specific differences. As depicted in FIG. 26D, re-weighting the connectivity matrix by regional expression of a gene-of-interest can allow for the evaluation of that gene's relevance in disease spread. Henderson et al. weighted a network diffusion model with SNCA expression, demonstrating that in silico simulation of circuits and genes can recover observed α-synuclein pathology (71).

Accurate whole-brain models of pathology dynamics that can predict both future and past states of these progressive disorders will have significant clinical implications. For example, a computational model that takes an arbitrary pathological state and predicts future states could guide interventions that depend on the predicted localization of pathology. Similarly, a model that can iterate backwards and compute previous states, even back to the initial seeding site, can further aid in disease progression classification for diagnosis purposes. Since neurodegenerative disorders like Parkinson's Disease, Lewy Body Dementia, and Multiple System Atrophy have long been hypothesized to collectively form a neuropathological spectrum (84), modeling the various circuits that drive different pathologies and clinical manifestations will play a large part in precisely defining these disorders.

While functional and histological readouts provide distinct information regarding the brain and disease, their colocalization is likely to be important in both furthering our understanding of neural circuitry and developing novel therapeutics. For instance, combining ofMRI and whole-brain pathological labeling could aid in the development of therapeutics that treat the underlying pathology, such as α-syn pathology in Parkinson's Disease. We give an example of spatial colocalization between optogenetic stimulation induced changes, and ofMRI-measured functional changes observed during the delivery of the same stimulation (FIGS. 26E and 26F). This optogenetic stimulation paradigm reduces the α-synuclein aggregate count at the site of stimulation and in various downstream regions. When colocalized with brain activity, their similar spatial locations but opposing polarities are apparent, revealing that positive activity is colocalized with decreases in pathology, while negative activity is highly colocalized with increases in pathology.

This ability to target pathology with neuromodulation, while predicting subsequent brain-wide changes in pathology could provide a new way of thinking about neuromodulation therapy for Parkinson's Disease and related disorders. For instance, parameters such as duration and site of neuromodulation could be tailored to patients based on their current pathological state and neuromodulation parameter's expected impact on pathology. Taking it one step further, therapy could even be designed while taking the expected future states of pathology into consideration. Altogether, the ability to readout and model pathology with brain clearing while measuring whole-brain network function with optogenetic fMRI will allow for the development of circuit-based models that bridge pathology and function.

### CONCLUSION

These recent advancements in cell-type-specific neuromodulation, whole-brain functional imaging, and computational modeling are starting to pave a path for a significant turning point in neuroscience. We aim to establish new approaches to simulating brain function that can replicate and predict behaviors of interest. This will transform treatments of a wide array of neurological diseases including Parkinson's Disease.

### REFERENCES

1. P. Calabresi, B. Picconi, A. Tozzi, V. Ghiglieri, M. Di Filippo, Direct and indirect pathways of basal ganglia: a critical reappraisal. Nat Neurosci 17, 1022-1030 (2014).
2. H. J. Lee et al., Activation of Direct and Indirect Pathway Medium Spiny Neurons Drives Distinct Brain-wide Responses. Neuron 91, 412-424 (2016).
3. J. H. Lee et al., Global and local fMRI signals driven by neurons defined optogenetically by type and wiring. Nature 465, 788-792 (2010).
4. D. Bernal-Casas, H. J. Lee, A. J. Weitz, J. H. Lee, Studying Brain Circuit Function with Dynamic Causal Modeling for Optogenetic fMRI. Neuron 93, 522-532 e525 (2017).
5. F. Zhang, A. M. Aravanis, A. Adamantidis, L. de Lecea, K. Deisseroth, Circuit-breakers: optical technologies for probing neural signals and systems. Nat Rev Neurosci 8, 577-581 (2007).
6. A. M. Aravanis et al., An optical neural interface: in vivo control of rodent motor cortex with integrated fiberoptic and optogenetic technology. J Neural Eng 4, S143-156 (2007).
7. Z. Fang, J. H. Lee, High-throughput optogenetic functional magnetic resonance imaging with parallel computations. J Neurosci Methods 218, 184-195 (2013).
8. Z. Fang, N. Le, M. Choy, J. H. Lee, High spatial resolution compressed sensing (HSPARSE) functional magnetic resonance imaging. Magn Reson Med In press, (2015).
9. D. J. Holland et al., Compressed Sensing Reconstruction Improves Sensitivity of Variable Density Spiral fMRI. Magnetic Resonance in Medicine 70, 1634-1643 (2013).
10. X. P. Zong, J. Y. Lee, A. J. Poplawsky, S. G. Kim, J. C. Ye, Compressed sensing fMRI using gradient-recalled echo and EPI sequences. Neuroimage 92, 312-321 (2014).
11. B. A. Duffy, M. Choy, M. R. Chuapoco, M. Madsen, J. H. Lee, MRI compatible optrodes for simultaneous LFP and optogenetic fMRI investigation of seizure-like afterdischarges. Neuroimage, (2015).
12. M. R. Chuapoco et al., Carbon monofilament electrodes for unit recording and functional MRI in same subjects. Neuroimage 186, 806-816 (2019).
13. B. Byers et al., Direct in vivo assessment of human stem cell graft-host neural circuits. Neuroimage 114, 328-337 (2015).
14. J. Liu et al., Frequency-selective control of cortical and subcortical networks by central thalamus. Elife 4, (2015).
15. A. J. Weitz et al., Optogenetic fMRI reveals distinct, frequency-dependent networks recruited by dorsal and intermediate hippocampus stimulations. Neuroimage 107, 229-241 (2015).
16. J. Liu et al., Frequency-selective control of cortical and subcortical networks by central thalamus. Elife 4, e09215 (2015).
17. A. J. Weitz, H. J. Lee, M. Choy, J. H. Lee, Thalamic Input to Orbitofrontal Cortex Drives Brain-wide, Frequency-Dependent Inhibition Mediated by GABA and Zona Incerta. Neuron 104, 1153-1167 e1154 (2019).
18. A. T. Leong et al., Long-range projections coordinate distributed brain-wide neural activity with a specific spatiotemporal profile. Proc Natl Acad Sci USA 113, E8306-E8315 (2016).
19. N. Takata et al., Optogenetic activation of CA1 pyramidal neurons at the dorsal and ventral hippocampus evokes distinct brain-wide responses revealed by mouse fMRI. PLoS One 10, e0121417 (2015).
20. W. B. Jung, H. Jiang, S. Lee, S. G. Kim, Dissection of brain-wide resting-state and functional somatosensory circuits by fMRI with optogenetic silencing. Proc Natl Acad Sci USA 119, (2022).
21. K. Y. Choe, C. F. Sanchez, N. G. Harris, T. S. Otis, P. J. Mathews, Optogenetic fMRI and electrophysiological identification of region-specific connectivity between the cerebellar cortex and forebrain. Neuroimage 173, 370-383 (2018).
22. E. A. Ferenczi et al., Prefrontal cortical regulation of brainwide circuit dynamics and reward-related behavior. Science 351, aac9698 (2016).
23. N. Takata et al., Optogenetic astrocyte activation evokes BOLD fMRI response with oxygen consumption without neuronal activity modulation. Glia 66, 2013-2023 (2018).
24. A. Gerits et al., Optogenetically induced behavioral and functional network changes in primates. Curr Biol 22, 1722-1726 (2012).
25. B. A. Duffy, M. Choy, J. H. Lee, Predicting Successful Generation and Inhibition of Seizure-like Afterdischarges and Mapping Their Seizure Networks Using fMRI. Cell Rep 30, 2540-2554 e2544 (2020).
26. M. Choy et al., Repeated hippocampal seizures lead to brain-wide reorganization of circuits and seizure propagation pathways. Neuron 110, 221-236 e224 (2022).
27. S. Vahdat et al., Brain-wide neural dynamics of poststroke recovery induced by optogenetic stimulation. Sci Adv 7, (2021).
28. B. Crompe et al., The globus pallidus orchestrates abnormal network dynamics in a model of Parkinsonism. Nat Commun 11, 1570 (2020).
29. K. Kondabolu et al., Striatal cholinergic interneurons generate beta and gamma oscillations in the corticostriatal circuit and produce motor deficits. Proc Natl Acad Sci USA 113, E3159-3168 (2016).
30. O. Yizhar et al., Neocortical excitation/inhibition balance in information processing and social dysfunction. Nature 477, 171-178 (2011).
31. Z. N. Fang, N. V. Le, M. Choy, J. H. Lee, High Spatial Resolution Compressed Sensing (HSPARSE) Functional MRI. Magnetic Resonance in Medicine 76, 440-455 (2016).
32. J. Demas et al., High-speed, cortex-wide volumetric recording of neuroactivity at cellular resolution using light beads microscopy. Nature Methods 18, 1103-+(2021).
33. V. Voleti et al., Real-time volumetric microscopy of in vivo dynamics and large-scale samples with SCAPE 2.0. Nature Methods 16, 1054-+(2019).
34. N. A. Steinmetz et al., Neuropixels 2.0: A miniaturized high-density probe for stable, long-term brain recordings. Science 372, (2021).
35. R. Hultman et al., Brain-wide Electrical Spatiotemporal Dynamics Encode Depression Vulnerability. Cell 173, 166-180 e114 (2018).
36. E. M. R. Lake et al., Simultaneous cortex-wide fluorescence Ca(2+) imaging and whole-brain fMRI. Nat Methods 17, 1262-1271 (2020).
37. O. Sporns, Networks of the brain. (The MIT Press, Cambridge, Massachusetts; London, England, ed. Paperback edition. 2016).
38. A. R. McIntosh, Towards a network theory of cognition. Neural Netw 13, 861-870 (2000).
39. X. J. Wang, Neurophysiological and Computational Principles of Cortical Rhythms in Cognition. Physiological Reviews 90, 1195-1268 (2010).
40. G. Deco, M. L. Kringelbach, Great Expectations: Using Whole-Brain Computational Connectomics for Understanding Neuropsychiatric Disorders. Neuron 84, 892-905 (2014).
41. A. Fornito, A. Zalesky, M. Breakspear, The connectomics of brain disorders. Nat Rev Neurosci 16, 159-172 (2015).
42. M. A. Tagamets, B. Horwitz, Integrating electrophysiological and anatomical experimental data to create a large-scale model that simulates a delayed match-to-sample human brain imaging study. Cereb Cortex 8, 310-320 (1998).
43. K. E. Stephan et al., Nonlinear dynamic causal models for fMRI. Neuroimage 42, 649-662 (2008).
44. S. J. Kiebel, O. David, K. J. Friston, Dynamic causal modelling of evoked responses in EEG/MEG with lead field parameterization. Neuroimage 30, 1273-1284 (2006).
45. R. L. Albin, A. B. Young, J. B. Penney, The functional anatomy of basal ganglia disorders. Trends Neurosci 12, 366-375 (1989).
46. M. R. DeLong, Primate models of movement disorders of basal ganglia origin. Trends Neurosci 13, 281-285 (1990).
47. J. G. Parker et al., Diametric neural ensemble dynamics in parkinsonian and dyskinetic states. Nature 557, 177-182 (2018).
48. K. J. Friston, L. Harrison, W. Penny, Dynamic causal modelling. Neuroimage 19, 1273-1302 (2003).
49. K. J. Friston, J. Kahan, B. Biswal, A. Razi, A DCM for resting state fMRI. Neuroimage 94, 396-407 (2014).
50. K. E. Stephan et al., Ten simple rules for dynamic causal modeling. Neuroimage 49, 3099-3109 (2010).
51. P. Salvan et al., Frequency modulation of entorhinal cortex neuronal activity drives distinct frequency-dependent states of brain-wide dynamics. Cell Rep 37, 109954 (2021).
52. S. Ryali et al., Combining optogenetic stimulation and fMRI to validate a multivariate dynamical systems model for estimating causal brain interactions. Neuroimage 132, 398-405 (2016).
53. C. Grimm et al., Optogenetic activation of striatal D1R and D2R cells differentially engages downstream connected areas beyond the basal ganglia. Cell Rep 37, 110161 (2021).
54. J. Kim et al., Inhibitory Basal Ganglia Inputs Induce Excitatory Motor Signals in the Thalamus. Neuron 95, 1181-1196 el188 (2017).
55. B. C. Schwab et al., Neural activity during a simple reaching task in macaques is counter to gating and rebound in basal ganglia-thalamic communication. PLoS Biol 18, e3000829 (2020).
56. J. H. Goldberg, M. A. Farries, M. S. Fee, Basal ganglia output to the thalamus: still a paradox. Trends Neurosci 36, 695-705 (2013).
57. G. Deco, V. K. Jirsa, P. A. Robinson, M. Breakspear, K. Friston, The dynamic brain: from spiking neurons to neural masses and cortical fields. PLoS Comput Biol 4, e1000092 (2008).
58. M. Schirner, A. R. McIntosh, V. Jirsa, G. Deco, P. Ritter, Inferring multi-scale neural mechanisms with brain network modelling. Elife 7, (2018).
59. J. J. J. Hjorth et al., The microcircuits of striatum in silico. Proc Natl Acad Sci USA 117, 9554-9565 (2020).
60. X. Zhang, S. Santaniello, Role of cerebellar GABAergic dysfunctions in the origins of essential tremor. Proc Natl Acad Sci USA 116, 13592-13601 (2019).
61. M. J. Bezaire, I. Raikov, K. Burk, D. Vyas, I. Soltesz, Interneuronal mechanisms of hippocampal theta oscillations in a full-scale model of the rodent CA1 circuit. Elife 5, (2016).
62. A. V. Kravitz et al., Regulation of parkinsonian motor behaviours by optogenetic control of basal ganglia circuitry. Nature 466, 622-626 (2010).
63. K. L. Eskow Jaunarajs, P. Bonsi, M. F. Chesselet, D. G. Standaert, A. Pisani, Striatal cholinergic dysfunction as a unifying theme in the pathophysiology of dystonia. Prog Neurobiol 127-128, 91-107 (2015).
64. G. Sciamanna et al., Optogenetic Activation of Striatopallidal Neurons Reveals Altered HCN Gating in DYT1 Dystonia. Cell Rep 31, 107644 (2020).
65. J. M. Meier et al., Virtual deep brain stimulation: Multiscale co-simulation of a spiking basal ganglia model and a whole-brain mean-field model with the virtual brain. Exp Neurol, 114111 (2022).
66. W. J. Neumann et al., Functional segregation of basal ganglia pathways in Parkinson's disease. Brain 141, 2655-2669 (2018).
67. E. M. Adam, E. N. Brown, N. Kopell, M. M. McCarthy, Deep brain stimulation in the subthalamic nucleus for Parkinson's disease can restore dynamics of striatal networks. Proc Natl Acad Sci USA 119, e2120808119 (2022).
68. S. Valverde et al., Deep brain stimulation-guided optogenetic rescue of parkinsonian symptoms. Nat Commun 11, 2388 (2020).
69. K. C. Luk et al., Pathological alpha-synuclein transmission initiates Parkinson-like neurodegeneration in nontransgenic mice. Science 338, 949-953 (2012).
70. Y. J. Guo, H. Xiong, K. Chen, J. J. Zou, P. Lei, Brain regions susceptible to alpha-synuclein spreading. Mol Psychiatry 27, 758-770 (2022).
71. M. X. Henderson et al., Spread of alpha-synuclein pathology through the brain connectome is modulated by selective vulnerability and predicted by network analysis. Nat Neurosci 22, 1248-1257 (2019).
72. K. Chung, K. Deisseroth, CLARITY for mapping the nervous system. Nat Methods 10, 508-513 (2013).
73. A. Erturk et al., Three-dimensional imaging of solvent-cleared organs using 3DISCO. Nat Protoc 7, 1983-1995 (2012).
74. N. Renier et al., iDISCO: a simple, rapid method to immunolabel large tissue samples for volume imaging. Cell 159, 896-910 (2014).
75. T. Liebmann et al., Three-Dimensional Study of Alzheimer's Disease Hallmarks Using the iDISCO Clearing Method. Cell Reports 16, 1138-1152 (2016).
76. N. Renier et al., Mapping of Brain Activity by Automated Volume Analysis of Immediate Early Genes. Cell 165, 1789-1802 (2016).
77. S. W. Oh et al., A mesoscale connectome of the mouse brain. Nature 508, 207-214 (2014).
78. E. S. Lein et al., Genome-wide atlas of gene expression in the adult mouse brain. Nature 445, 168-176 (2007).
79. C. Kirst et al., Mapping the Fine-Scale Organization and Plasticity of the Brain Vasculature. Cell 180, 780-795 e725 (2020).
80. S. Pandya et al., Predictive model of spread of Parkinson's pathology using network diffusion. Neuroimage 192, 178-194 (2019).
81. A. Raj, A. Kuceyeski, M. Weiner, A network diffusion model of disease progression in dementia. Neuron 73, 1204-1215 (2012).
82. S. Pandya, C. Mezias, A. Raj, Predictive Model of Spread of Progressive Supranuclear Palsy Using Directional Network Diffusion. Frontiers in Neurology 8, (2017).
83. G. Bieri et al., LRRK2 modifies alpha-syn pathology and spread in mouse models and human neurons. Acta Neuropathol 137, 961-980 (2019).
84. J. W. Langston, The Parkinson's complex: Parkinsonism is just the tip of the iceberg. Annals of Neurology 59, 591-596 (2006).

What is claimed is:

1. A computer implemented method for predicting locations where pathological protein aggregates will develop in the brain of a subject who has a neurological or a neurodegenerative disease, the computer performing steps comprising:
a) receiving an image of the brain of the subject;
b) identifying pathological protein aggregates in the image using a machine learning algorithm;
c) mapping positions of the pathological protein aggregates to neuroanatomical regions;
d) modeling a discretized distribution of the pathological protein aggregates in each neuroanatomical region as a set of differential equations using a Smoluchowski network model;
e) using regional gene density maps for each neuroanatomical region to calculate a distribution of gene effects on regional spreading and decay of the pathological protein aggregates;
f) predicting changes in regional density of the pathological protein aggregates as a function of time by modeling spreading, aggregation, decay, and the spatial gene expression of the pathological protein aggregates throughout the neuroanatomical regions, wherein spreading is assumed to occur retrogradely between anatomically interconnected neuroanatomical regions, wherein spreading is modeled as diffusion through a weighted directed graph connecting the neuroanatomical regions of the brain of the subject; and
g) predicting past locations, present locations, and future locations of the pathological protein aggregates based on said modeling.

2. The computer implemented method of claim 1, further comprising adjusting one or more programmed neurostimulation parameters based on said predicting the changes in regional density of the pathological protein aggregates as a function of time.

3. The computer implemented method of claim 2, wherein the one or more programmed neurostimulation parameters are selected from duration, amplitude, frequency, pulse width, and location of the neurostimulation.

4. The computer implemented method of any one of claims 1-3, further comprising instructing a neurostimulation device to apply neurostimulation to locations of the brain where pathological protein aggregates are predicted to be present in order to treat the neurological or neurodegenerative disease in the subject.

5. The computer implemented method of claim 4, further comprising instructing the neurostimulation device to apply electrical stimulation to locations of the brain where pathological protein aggregates are not yet present but are predicted to occur at a future time based on said predicting changes in the regional density of the pathological protein aggregates as a function of time.

6. The computer implemented method of any one of claims 1-5, further comprising:
performing image registration to a coordinate space comprising a plurality of voxels, wherein each voxel is represented as a cubic volumetric element centered at a coordinate in the coordinate space;
identifying positions in x, y, z coordinates of each pathological protein aggregate in the coordinate space;
measuring volumes of each pathological protein aggregate from total number of voxels occupied by each pathological protein aggregate;
calculating aggregate density for each voxel, wherein the aggregate density for each voxel is determined from total number of pathological protein aggregates having centers within the same voxel;
calculating total aggregate size for each voxel, wherein the total aggregate size is the total size of all the pathological protein aggregates having centers within the same voxel;
calculating mean aggregate size for each voxel as the total aggregate size divided by the aggregate density for each voxel;
calculating total signal intensity for each voxel from the total intensity of all the pathological protein aggregates having centers within the same voxel; and
calculating mean signal intensity for each voxel as the total signal intensity divided by the aggregate density for each voxel.

7. The computer implemented method of any one of claims 1-6, wherein said modeling the discretized distribution of the pathological protein aggregates in each neuroanatomical region as a set of differential equations using a Smoluchowski network model comprises using the following set of differential equations:
$$\frac{d {c}_{1 , j}}{dt} = - α {\sum }_{k = 1}^{V} {L}_{jk} {c}_{1 , k} - {μ}_{1 , j} {c}_{1 , j} - 2 {c}_{1 , j}^{2} ( {κ}_{j} + ξ {\sum }_{k = 2}^{N} k {c}_{2 , k} ) - {c}_{1 , j} {\sum }_{k = 2}^{N} {c}_{k , j} , \\ \frac{d {c}_{2 , j}}{dt} = - {2}^{- η} α {\sum }_{k = 1}^{V} {L}_{jk} {c}_{2 , k} - {2}^{λ} {μ}_{2 , j} {c}_{2 , j} + {c}_{1 , j}^{2} ( {κ}_{j} + ξ {\sum }_{k = 2}^{N} {kc}_{2 , k} ) - {c}_{1 , j} {c}_{2 , j} , \\ \frac{d {c}_{i , j}}{dt} = - {i}^{- η} α {\sum }_{k = 1}^{V} {L}_{jk} {c}_{i , k} - {i}^{λ} {μ}_{ij} {c}_{ij} + {c}_{1 , j} ( {c}_{i - 1 , j} - ci , j ) , and \\ \frac{d {c}_{N , j}}{dt} = - {N}^{λ} {μ}_{N , j} {c}_{N , j} + {c}_{1 , j} {c}_{N - 1 , j} ,$$
wherein c_{i,j} represents the total count of pathological protein aggregates in a discretized size-bin indexed by l, in a brain region indexed by j, wherein an L matrix represents the Laplacian matrix of the weighted directed graph connecting the neuroanatomical regions of the brain, wherein η is chosen as a hyperparameter that slows the spread of large aggregates as the inverse power of the size, and wherein λ is chosen as a hyperparameter that accelerates the decay of pathological protein aggregates proportionally to the power of their size.

8. The computer implemented method of claim 7, wherein initial values for α and μ are fit by sweeping through a 2-dimensional-grid and selecting values that result in a lowest mean-squared error between predicted and actual counts of the pathological protein aggregates.

9. The computer implemented method of claim 7, wherein the initial value of μ=0, the initial value of k=0, ξ=0, and c is a one-dimensional size vector, wherein the set of differential equations simplifies to a standard network diffusion model.

10. The computer implemented method of any one of claims 7-9, further comprising quantifying sensitivity of the model to specific neuroanatomical pathways in the brain, wherein a Jacobian matrix is calculated by taking a partial derivative of the model's output with respect to the weight of the anatomical connection strength between two neuroanatomical regions encoded into the model, wherein an element of the Jacobian matrix represents relative contribution of the anatomical connection between the two neuroanatomical regions to the spreading of pathological protein aggregates to a specific region of the brain.

11. The computer implemented method of any one of claims 7-10, further comprising using the model to produce a ranking of candidate seed locations for a given pathological state c at t=T months post-injection (MPI) by a method comprising:
using each of the neuroanatomical regions as separate seed locations at t=0 within the model and simulated forward in time to t=T MPI, wherein each of the simulation results for the different neuroanatomical regions are compared with an observed state c using a pairwise similarity metric, wherein the similarity metric is a correlation coefficient between total regional aggregate counts across observed and simulated states; and
using the similarity metric values to sort the seed locations for the neuroanatomical regions as likely sites that lead to the observed pathological state c, wherein the ranking of candidate seed locations for the given pathological state c at t=T MPI is produced.

12. The computer implemented method of claim 11, further comprising predicting the time since seeding t=T MPI for a given pathological state c by a method comprising:
comparing the whole-brain distribution of aggregate sizes for state c with simulated distributions at various t using a pairwise similarity metric without taking the seed location into account, wherein the distribution of simulated aggregate sizes across the whole brain is assumed to be invariant with respect to which neuroanatomical region is used as the seed location at t=0; and
calculating the mean squared error between the stimulated and observed distributions, wherein when deciding among several candidate t values, the mean squared errors are inverted and normalized to sum to 1 to provide a prediction probability for each t being the correct estimate of T for the given pathological state c.

13. The computer implemented method of any one of claims 7-12, wherein the gene effects on regional spreading and decay of the pathological protein aggregates are determined by a method comprising:
assuming that α (spreading) and μ (decay) parameters are regionally dependent, wherein the spreading from a specific neuroanatomical region is proportional to the gene density in that region;
normalizing all genes to the same range so that only the regional distribution of gene expression relative to that gene's total whole-brain expression is compared, wherein a is a vector, and the product of a with the Laplacian connectivity matrix L has the effect of modifying the regional connectivity encoded into the model; and
normalizing each gene vector to have a mean of 1 and a standard deviation I that is empirically set to preserve a correlation between predicted and observed whole-brain aggregate count, wherein the normalization is chosen so that the product has the effect of maintaining the trace of the original Laplacian connectivity matrix L.

14. The computer implemented method of claim 13, wherein derivation of the normalization for maintaining the trace of the original Laplacian connectivity matrix L comprises:
assuming the vector s is sampled from a multivariate normal distribution with mean-value 1 and standard deviation Σ, wherein s˜N(1,Σ);
using a definition of the matrix trace and representing s as a diagonal square matrix S, wherein the trace of the product of S and the Laplacian connectivity matrix L results in the following:
$$Tr ( SL ) = Tr ( [ \begin{matrix}{s}_{1} & ⋯ & 0 \\ ⋮ & ⋱ & ⋮ \\ 0 & ⋯ & {s}_{V}\end{matrix} ] L ) = {\sum }_{i = 1}^{V} {s}_{i} {L}_{ii} = s \cdot diag ( L ) = s \cdot l ,$$
wherein l represents the diagonal of L, and wherein the trace is equivalent to the dot product of s and l, which has an expectation value equivalent to the sum of the entries of l, which recovers the definition of the trace of L according to the following equations:
$$s \cdot l ∼ N ( 1 \cdot l , l \sum l ) \\ E [ s \cdot l ] = Tr ( L ) ,$$
wherein after each gene is encoded into the model;
comparing net effects on the regional correlation between the simulated and actual data to the baseline correlation with no genes; and
providing an ordered list of genes ranked by the relevance of their spatial expression map in improving the regional predictions of the model.

15. The computer implemented method of any one of claims 1-14, wherein the cubic volumetric element has a width of 100 μm in the coordinate space.

16. The computer implemented method of any one of claims 1-15, wherein one or more pathological protein aggregates map to a single voxel.

17. The computer implemented method of any one of claims 1-16, further comprising performing multidimensional Gaussian filtering to account for variations in image registration between different samples.

18. The computer implemented method of any one of claims 1-17, further comprising segmenting the image to produce a plurality of image segments.

19. The computer implemented method of any one of claims 1-18, wherein said mapping comprises mapping the locations of the pathological protein aggregates to neuroanatomical regions of the Allen Human Brain Reference Atlas.

20. The computer implemented method of claim 19, wherein said mapping comprises performing image registration to transform the locations of the pathological protein aggregates to the Allen Human Brain Reference Atlas coordinate space.

21. The computer implemented method of claim 19 or 20, wherein anatomically interconnected neuroanatomical regions are identified from the Allen Connectivity Atlas.

22. The computer implemented method of any one of claims 1-21, wherein the neuroanatomical regions are selected from an Anterior amygdalar area, Anterior cingulate area, dorsal part, Anterior cingulate area, ventral part, Nucleus accumbens, Anterodorsal nucleus, Anterior hypothalamic nucleus, Agranular insular area, dorsal part, Agranular insular area, posterior part, Agranular insular area, ventral part, Nucleus ambiguous, Anteromedial nucleus, dorsal part, Anteromedial nucleus, ventral part, Ansiform lobule, Accessory olfactory bulb, Anterior olfactory nucleus, Anterior pretectal nucleus, Arcuate hypothalamic nucleus, Dorsal auditory area, Primary auditory area, Ventral auditory area, Anteroventral nucleus of thalamus, Basolateral amygdalar nucleus, Basomedial amygdalar nucleus, Bed nuclei of the stria terminalis, Field CA1, Field CA2, Field CA3, Central amygdalar nucleus, Central lobule, Central lateral nucleus of the thalamus, Claustrum, Central linear nucleus raphe, Central medial nucleus of the thalamus, Cortical amygdalar area, anterior part, Cortical amygdalar area, posterior part, Caudoputamen, Superior central nucleus raphe, Culmen, Cuneiform nucleus, Dorsal cochlear nucleus, Dentate gyrus, Dorsomedial nucleus of the hypothalamus, Dentate nucleus, Dorsal peduncular area, Dorsal nucleus raphe, Ectorhinal area, Entorhinal area, lateral part, Entorhinal area, medial part, dorsal zone, Endopiriform nucleus, dorsal part, Endopiriform nucleus, ventral part, Flocculus, Fastigial nucleus, Frontal pole, cerebral cortex, Fundus of striatum, Globus pallidus, external segment, Globus pallidus, internal segment, Gigantocellular reticular nucleus, Gustatory areas, Intercalated amygdalar nucleus, Inferior colliculus, central nucleus, Inferior colliculus, dorsal nucleus, Inferior colliculus, external nucleus, Infralimbic area, Intermediodorsal nucleus of the thalamus, Inferior olivary complex, Interposed nucleus, Interpeduncular nucleus, Intermediate reticular nucleus, Lateral amygdalar nucleus, Lateral vestibular nucleus, Lateral dorsal nucleus of thalamus, Dorsal part of the lateral geniculate complex, Ventral part of the lateral geniculate complex, Lateral habenula, Lateral hypothalamic area, Lateral posterior nucleus of the thalamus, Lateral preoptic area, Lateral reticular nucleus, Lateral septal nucleus, caudal (caudodorsal) part, Lateral septal nucleus, rostral (rostroventral) part, Lateral septal nucleus, ventral part, Magnocellular nucleus, Magnocellular reticular nucleus, Mediodorsal nucleus of thalamus, Medullary reticular nucleus, dorsal part, Medullary reticular nucleus, ventral part, Medial amygdalar nucleus, Median preoptic nucleus, Medial geniculate complex, dorsal part, Medial geniculate complex, medial part, Medial geniculate complex, ventral part, Medial habenula, Medial mammillary nucleus, Main olfactory bulb, Primary motor area, Secondary motor area, Medial preoptic nucleus, Medial preoptic area, Medial pretectal area, Midbrain reticular nucleus, Medial septal nucleus, Medial vestibular nucleus Diagonal band nucleus, Nucleus incertus, Nucleus of the lateral lemniscus, Nucleus of the lateral olfactory tract, Nodulus (X), Nucleus of the optic tract, Nucleus of the posterior commissure, Nucleus of the solitary tract, Orbital area, lateral part, Orbital area, medial part, Orbital area, ventrolateral part, Olfactory tubercle, Posterior amygdalar nucleus, Piriform-amygdalar area, Periaqueductal gray, Parasubiculum, Parvicellular reticular nucleus, Parabrachial nucleus, Pontine central gray, Perirhinal area, Parafascicular nucleus, Paraflocculus, Pontine gray, Paragigantocellular reticular nucleus, dorsal part, Paragigantocellular reticular nucleus, lateral part, Posterior hypothalamic nucleus, Piriform area, Prelimbic area, Dorsal premammillary nucleus, Posterior complex of the thalamus, Posterior limiting nucleus of the thalamus, Postsubiculum, Peripeduncular nucleus, Pedunculopontine nucleus, Presubiculum, Paramedian lobule, Pontine reticular nucleus, caudal part, Pontine reticular nucleus, Nucleus prepositus, Principal sensory nucleus of the trigeminal, Parataenial nucleus, Posterior parietal association areas, Paraventricular hypothalamic nucleus, Paraventricular nucleus of the thalamus, Periventricular hypothalamic nucleus, posterior part, Periventricular hypothalamic nucleus, preoptic part, Pyramus (VIII), Retrochiasmatic area, Nucleus of reuniens, Rhomboid nucleus, Nucleus raphe magnus, Red nucleus, Midbrain reticular nucleus, retrorubral area, Retrosplenial area, lateral agranular part, Retrosplenial area, dorsal part, Retrosplenial area, ventral part, Reticular nucleus of the thalamus, Subparaventricular zone, Superior colliculus, motor related, Superior colliculus, sensory related, Septofimbrial nucleus, Substantia innominate, Simple lobule, Submedial nucleus of the thalamus, Substantia nigra, compact part, Substantia nigra, reticular part, Superior olivary complex, Subparafascicular area, Subparafascicular nucleus, magnocellular part, Subparafascicular nucleus, parvicellular part, Spinal vestibular nucleus, Spinal nucleus of the trigeminal, caudal part, Spinal nucleus of the trigeminal, interpolar part, Spinal nucleus of the trigeminal, oral part, Primary somatosensory area, barrel field, Primary somatosensory area, lower limb, Primary somatosensory area, mouth, Primary somatosensory area, nose, Primary somatosensory area, trunk, Primary somatosensory area, upper limb, Supplemental somatosensory area, Subthalamic nucleus, Subiculum, Supramammillary nucleus, Supratrigeminal nucleus, Superior vestibular nucleus, Temporal association areas, Postpiriform transition area, Tegmental reticular nucleus, Triangular nucleus of septum, Taenia tecta, Tuberal nucleus, Motor nucleus of trigeminal, Ventral anterior-lateral complex of the thalamus, Ventral cochlear nucleus, Facial motor nucleus, Visceral area, Anterolateral visual area, Anteromedial visual area, Lateral visual area, Primary visual area, Posterolateral visual area, posteromedial visual area, Ventral medial nucleus of the thalamus, Ventromedial hypothalamic nucleus, Ventral posterolateral nucleus of the thalamus, Ventral posteromedial nucleus of the thalamus, Ventral posteromedial nucleus of the thalamus, parvicellular part, Ventral tegmental area, and Hypoglossal nucleus.

23. The computer implemented method of any one of claims 1-22, further comprising predicting where pathological protein aggregates originated in the brain of the subject.

24. The computer implemented method of any one of claims 1-23, wherein the machine learning algorithm uses an artificial neural network.

25. The computer implemented method of any one of claims 1-24, wherein the machine learning algorithm uses a deep learning algorithm.

26. The computer implemented method of claim 25, wherein the deep learning algorithm uses a convolutional neural network, a deep neural network, a recurrent neural network, a deep residual neural network, a long short-term memory network, a deep belief network, a multilayer perceptron, or deep reinforcement learning.

27. The computer implemented method of any one of claims 1-26, wherein the machine learning algorithm is supervised, semi-supervised, or unsupervised.

28. The computer implemented method of any one of claims 1-27, wherein the subject is a human subject.

29. The computer implemented method of claim 28, wherein modeling spreading, aggregation, and decay of the pathological protein aggregates in the brain of the human subject uses a simulation generated based on measuring spreading, aggregation, and decay of the pathological protein aggregates in a non-human animal model, wherein pathology measured in the non-human animal model experimentally is used to design simulation parameters to predict the past locations, the present locations, and the future locations of the pathological protein aggregates in the human subject.

30. The computer implemented method of claim 29, wherein the simulation further uses brain anatomy or brain function measured from the non-human animal to predict the past locations, the present locations, and the future locations of the pathological protein aggregates in the human subject.

31. The computer implemented method of claim 29 or 30, wherein the non-human animal is a mammal.

32. The computer implemented method of claim 31, wherein the mammal is a rodent or a primate.

33. The computer implemented method of claim 32, wherein the rodent is a mouse.

34. The computer implemented method of any one of claims 29-33, wherein the simulation further uses brain anatomy or brain function measured from other human subjects to design simulation parameters to predict the past locations, the present locations, and the future locations of the pathological protein aggregates in the human subject.

35. The computer implemented method of any one of claims 1-34, further comprising:
receiving a second image of the brain, wherein the second image is taken after said neurostimulation is applied to the brain;
repeating steps (b)-(o) using the second image; and
displaying changes in the total aggregate size for each voxel, the volume of each pathological protein aggregate for each voxel, and the aggregate density for each voxel in the image taken after said neurostimulation is applied to the brain of the subject compared to the image taken before said neurostimulation is applied to the brain of the subject.

36. The computer implemented method of any one of claims 1-34, further comprising:
receiving a second image of the brain, wherein the second image is taken after said neurostimulation is applied to the brain;
repeating steps (b)-(o) using the second image;
modulating one or more programmed neurostimulation parameters based on any changes in the past locations, present locations, or future locations where the pathological protein aggregates are predicted to occur; and
instructing a neurostimulation device to apply modulated neurostimulation to the brain of the subject in order to treat the neurological or neurodegenerative disease in the subject.

37. The computer implemented method of any one of claims 1-36, further comprising storing a user profile for the subject comprising information regarding the programmed neurostimulation parameters used to apply neurostimulation to the brain of the subject to treat the neurological or neurodegenerative disease based on where the computer implemented method predicts pathological protein aggregates are present or will develop at a future time.

38. A non-transitory computer-readable medium comprising program instructions that, when executed by a processor in a computer, causes the processor to perform the method of any one of claims 1-37.

39. A kit comprising the non-transitory computer-readable medium of claim 38 and instructions for treating a neurological or a neurodegenerative disease in a subject with neurostimulation.

40. A method for treating a neurological or neurodegenerative disease in a subject, the method comprising:
imaging pathological protein aggregates in the brain of the subject;
using the computer implemented method of any one of claims 1-37 to predict where pathological protein aggregates will develop based on locations of the pathological protein aggregates that are detected in the brain of the subject by said imaging; and
applying neurostimulation at locations in the brain where the pathological protein aggregates are detected in the brain of the subject by said imaging and at locations where the computer implemented method predicts pathological protein aggregates will develop.

41. The method of claim 40, wherein said imaging is performed using computed tomography (CT), single photon emission computed tomography (SPECT), magnetic resonance imaging, functional magnetic resonance imaging, optogenetic functional magnetic resonance imaging, or positron emission tomography (PET).

42. The method of claim 40 or 41, further comprising adjusting stimulation frequency and pulse width of the neurostimulation to target specific neuronal cell-types or circuits within the brain at the locations in the brain where the computer implemented method predicts the pathological protein aggregates are present or will develop at the future time.

43. The method of any one of claims 40-42, wherein the neurological or neurodegenerative disease is a synucleinopathy.

44. The method of claim 43, wherein the synucleinopathy is Parkinson's disease, dementia with Lewy bodies, multiple system atrophy, neuroaxonal dystrophy Shy-Drager syndrome, striatonigral degeneration, or olivopontocerebellar atrophy.

45. The method of any one of claims 40-42, wherein the neurological or neurodegenerative disease is Alzheimer's disease, amyotrophic lateral sclerosis, or frontotemporal dementia.

46. The method of any one of claims 40-45, wherein the pathological protein aggregates comprise alpha-synuclein aggregates.

47. The method of any one of claims 40-46, wherein said applying neurostimulation comprises applying neurostimulation using an electrode.

48. The method of claim 47, wherein the electrode is a depth electrode or a surface electrode.

49. The method of claim 47, wherein the electrode is a non-brain penetrating surface electrode array or a brain-penetrating electrode array.

50. The method of any one of claims 40-49, wherein said applying neurostimulation comprises applying deep brain stimulation, transcranial magnetic stimulation, or transcranial electrical stimulation.

51. The method of any one of claims 40-46, wherein said applying neurostimulation comprises applying neurostimulation optogenetically.

52. The method of claim 51, wherein neurostimulation is applied optogenetically by a method comprising:
introducing a recombinant polynucleotide encoding a light-responsive ion channel into a neuron at the location in the brain where the computer implemented method predicts pathological protein aggregates are present or will develop at a future time, wherein the light-responsive ion channel is expressed in the neuron; and
illuminating the light-responsive ion channel with light at a wavelength that activates the light-responsive ion channel, wherein conduction of ions by the light-responsive ion channel in response to absorption of light results in hyperpolarization or depolarization of the neuron.

53. The method of claim 52, wherein the light-responsive ion channel is a light-responsive anion-conducting opsin or a light-responsive proton conductance regulator.

54. The method of claim 53, wherein the light-responsive anion-conducting opsin conducts chloride ions (Cl⁻).

55. The method of claim 53 or 54, wherein the anion-conduction opsin is an anion-conducting channelrhodopsin or halorhodopsin.

56. The method of claim 55, wherein the halorhodopsin is a Natronomonas pharaonis halorhodopsin (NpHR), enhanced NpHR (eNpHR) 1.0, eNpHR 2.0, or eNpHR 3.0.

57. The method of claim 55, wherein the anion-conducting channelrhodopsin is iC1C2, SwiChR, SwiChR++, or iC++.

58. The method of claim 53, wherein the light-responsive proton conductance regulator is a bacteriorhodopsin or an archaerhodopsin.

59. The method of claim 58, wherein the light-responsive proton conductance regulator is Arch from Halorubrum sodomense, ArchT from Halorubrum sp., TP009 from Leptosphaeria maculans, or Mac from Leptosphaeria maculans.

60. The method of claim 52, wherein the light-responsive ion channel is a light-responsive cation-conducting opsin.

61. The method of claim 60, wherein the light-responsive cation-conducting opsin conducts calcium cations (Ca²⁺).

62. The method of claim 60 or 61, wherein the light-responsive cation-conducting opsin is a light-responsive cation-conducting channelrhodopsin.

63. The method of claim 62, wherein the light-responsive cation-conducting channelrhodopsin is a Chlamydomonas reinhardtii channelrhodopsin or a Volvox carteri channelrhodopsin.

64. The method of claim 63, wherein the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

65. The method of any one of claims 52-64, wherein the polynucleotide encoding the light-responsive ion channel is provided by a viral vector.

66. The method of claim 65, wherein the viral vector is a lentiviral vector or an adeno-associated viral (AAV) vector.

67. The method of claim 65 or 66, wherein the viral vector is stereotactically injected into the brain at the location where the computer implemented method predicts pathological protein aggregates are present or will develop at a future time.

68. The method of any one of claims 65-67, wherein the vector further comprises a neuron-specific promoter operably linked to the polynucleotide encoding the light-responsive ion channel.

69. The method of any one of claims 52-68, wherein expression of the light-responsive ion channel is inducible.

70. The method of any one of claims 52-69, wherein said illuminating the light-responsive ion channel comprises delivering light from a light source to the light-responsive ion channel using a fiber-optic-based optical neural interface.

71. The method of claim 70, wherein the light source is a solid-state diode laser.

72. The method of any one of claims 52-71, wherein said applying neurostimulation comprises applying neurostimulation to a motor cortex region or a subcortical region of the brain.

73. The method of any one of claims 40-72, wherein multiple cycles of the neurostimulation are performed.

74. The method of any one of claims 40-73, further comprising assessing effectiveness of the treatment of the neurological or neurodegenerative disease in the subject.

75. The method of claim 74, wherein said assessing comprises imaging the brain of the subject to measure sizes and identify locations of the pathological protein aggregates after said neurostimulation.

76. The method of claim 74 or 75, wherein said assessing comprises measuring brain function of the subject after said neurostimulation.

77. The method of claim 76, wherein said measuring brain function comprises performing electroencephalography (EEG), stereoelectroencephalography (sEEG), electrocorticography (ECoG), magnetoencephalography (MEG), single photon emission computed tomography (SPECT), functional magnetic resonance imaging (fMRI), optogenetic functional magnetic resonance imaging, or positron emission tomography (PET).

78. The method of claim 76 or 77, further comprising modulating one or more programmed neurostimulation parameters to improve the brain function.

79. The method of any one of claims 74-78, further comprising assessing severity of symptoms of the neurological or neurodegenerative disease using a visual analog scale, a verbal rating scale, a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale

80. A system for treating a neurological or neurodegenerative disease in a subject, the system comprising:
a neurostimulation device; and
a processor programmed according to the computer implemented method of any one of claims 1-37 to instruct the neurostimulation device to deliver neurostimulation to the brain of the subject in a manner effective to treat the neurological or neurodegenerative disease in the subject, wherein neurostimulation is applied to the brain at predicted present locations of the pathological protein aggregates, at predicted future locations of the pathological protein aggregates, or at predicted past locations of the pathological protein aggregates, or a combination thereof.

81. The system of claim 80, wherein the neurostimulation device comprises an electrode.

82. The system of claim 81, wherein the electrode is a depth electrode or a surface electrode.

83. The system of claim 82, wherein the electrode is a non-brain penetrating surface electrode array or a brain-penetrating electrode array.

84. The system of any one of claims 80-83, wherein the neurostimulation device performs deep brain stimulation, transcranial magnetic stimulation, or transcranial electrical stimulation.

85. The system of any one of claims 80-84, further comprising a display.

86. The system of claim 85, wherein the display displays an image of the brain of the subject showing the predicted present locations, past locations, or future locations of the pathological protein aggregates determined by the computer implemented method.

87. The system of claim 85 or 86, wherein the display displays information regarding the coordinates of each pathological protein aggregate, the volume of each pathological protein aggregate; the aggregate density for each voxel, the total aggregate size for each voxel, the mean aggregate size for each voxel, the total signal intensity for each voxel, the mean signal intensity for each voxel, or the mapping of the positions of the pathological protein aggregates to neuroanatomical regions, or any combination thereof.

88. The system of any one of claims 85-87, wherein the display displays information regarding the distribution of gene effects on regional spreading and decay of the pathological protein aggregates.

89. The system of any one of claims 85-88, wherein the display displays information regarding the predicted changes in regional density of the pathological protein aggregates as a function of time determined by the modeling of the spreading, aggregation, decay, and the spatial gene expression of the pathological protein aggregates throughout the neuroanatomical regions.

90. The system of any one of claims 85-89, wherein the display displays information regarding the predicted past locations, present locations, and future locations of the pathological protein aggregates based on the modeling.

91. The system of any one of claims 85-90, wherein the system further comprises a user interface comprising an input electronically coupled to the processor for instructing the neurostimulation device to apply neurostimulation to the brain of the subject to treat the neurological or neurodegenerative disease in the subject.

92. The system of claim 91, wherein the user interface is password protected and is operable by a health care practitioner.

93. The system of any one of claims 85-92, wherein the neurological or neurodegenerative disease is a synucleinopathy.

94. The system of claim 93, wherein the synucleinopathy is Parkinson's disease, dementia with Lewy bodies, multiple system atrophy, neuroaxonal dystrophy Shy-Drager syndrome, striatonigral degeneration, or olivopontocerebellar atrophy.

95. The system of any one of claims 85-92, wherein the neurological or neurodegenerative disease is Alzheimer's disease, amyotrophic lateral sclerosis, or frontotemporal dementia.
